SEQUENCE LISTING (42 sequences)

SEQ ID NO: 1 - DNA, 23 bp, Homo Sapiens
cagtacctgccagatacagacga

SEQ ID NO: 2 - DNA, 17 bp, Homo Sapiens
gggaaggcacagcaatg

SEQ ID NO: 3 - DNA, 27 bp, Homo Sapiens
tgatgatttactagcacaaggagacat

SEQ ID NO: 4 - DNA, 22 bp, Homo Sapiens
tgtacccccagagacctcatag

SEQ ID NO: 5 - DNA, 22 bp, Homo Sapiens
ctgatgtcaatgctcaggagcc

SEQ ID NO: 6 - DNA, 26 bp, Homo Sapiens
tgtgtcatagctctcctcatcctcac

SEQ ID NO: 7 - DNA, 19 bp, Homo Sapiens
ctgaaaactacgagggcca

SEQ ID NO: 8 - DNA, 19 bp, Homo Sapiens
cctccactgccaaatgaag

SEQ ID NO: 9 - DNA, 21 bp, Homo Sapiens
cccagggacctctctctaatc

SEQ ID NO: 10 - DNA, 21 bp, Homo Sapiens
atgggctacaggcttgtcact

SEQ ID NO: 11 - DNA, 20 bp, Homo Sapiens
cccatgcaggcacttactac

SEQ ID NO: 12 - DNA, 23 bp, Homo Sapiens
acgtcttcttgaacctcagaaca

SEQ ID NO: 13 - DNA, 19 bp, Homo Sapiens
cggacagggtctacatcaa

SEQ ID NO: 14 - DNA, 19 bp, Homo Sapiens
cttaggcgaaggtggagtt

SEQ ID NO: 15 - DNA, 25 bp, Homo Sapiens
cccatttcgacgatgacgagttgtg

SEQ ID NO: 16 - DNA, 25 bp, Homo Sapiens
ggagtaggattggccttggaagatg

SEQ ID NO: 17 - DNA, 2024 bp, Homo Sapiens
aaggcaagagatctaggacttctagcccctgaactttcagccgaatacatcttttccaaaggagtgaattcaggcccttgtatcactggcagcaggacgtgaccatggagaagctgttgtgtttcttggtcttgaccagcctctctcatgcttttggccagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctgaggcccagctgtgggtcctgaaggtacctcccggttttttacaccgcatgggccccacgtctctgtctctggtacctcccgcttttttacactgcatggttcccacgtctctgtctctgggcctttgttcccctatatgcattgcaggcctgctccaccctcctcagcgcctgagaatggaggtaaagtgtctggtctgggagctcgttaactatgctgggaaacggtccaaaagaatcagaatttgaggtgttttgttttcatttttatttcaagttggacagatcttggagataatttcttacctcacatagatgagaaaactaacacccagaaaggagaaatgatgttataaaaaactcataaggcaagagctgagaaggaagcgctgatcttctatttaattccccacccatgacccccagaaagcaggagggcattgcccacattcacagggctcttcagtctcagaatcaggacactggccaggtgtctggtttgggtccagagtgctcatcatcatgtcatagaactgctgggcccaggtctcctgaaatgggaagcccagcaataccacgcagtccctccactttctcaaagcacactggaaaggccattagaattgccccagcagagcagatctgctttttttccagagcaaaatgaagcactaggtataaatatgttgttactgccaagaacttaaatgactggtttttgtttgcttgcagtgctttcttaattttatggctcttctgggaaactcctccccttttccacacgaaccttgtggggctgtgaattctttcttcatccccgcattcccaatatacccaggccacaagagtggacgtgaaccacagggtgtcctgtcagaggagcccatctcccatctccccagctccctatctggaggatagttggatagttacgtgttcctagcaggaccaactacagtcttcccaaggattgagttatggactttgggagtgagacatcttcttgctgctggatttccaagctgagaggacgtgaacctgggaccaccagtagccatcttgtttgccacatggagagagactgtgaggacagaagccaaactggaagtggaggagccaagggattgacaaacaacagagccttgaccacgtggagtctctgaatcagccttgtctggaaccagatctacacctggactgcccaggtctataagccaataaagcccctgtttacttgaaaaaaaaaa

SEQ ID NO: 18 - DNA, 760 bp, Homo Sapiens
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttgaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtacaaaaaaaaaaaaaaa

SEQ ID NO: 19 - DNA, 2387 bp, Homo Sapiens
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagcggctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaacctttaaaaaaaaaaaaaaaaaaaaaaaaaaaaaaaaaaaaaaaaaaaaaaaaaaaa

SEQ ID NO: 20 - DNA, 1669 bp, Homo Sapiens
ctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaa

SEQ ID NO: 21 - DNA, 1201 bp, Homo Sapiens
aatattagagtctcaacccccaataaatataggactggagatgtctgaggctcattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaaaaaaaaaaa

SEQ ID NO: 22 - DNA, 561 bp, Homo Sapiens
accacagtggtgtccgagaagtcaggcacgtagctcagcggcggccgcggcgcgtgcgtctgtgcctctgcgcgggtctcctggtccttctgccatcatgccgatgttcatcgtaaacaccaacgtgccccgcgcctccgtgccggacgggttcctctccgagctcacccagcagctggcgcaggccaccggcaagcccccccagtacatcgcggtgcacgtggtcccggaccagctcatggccttcggcggctccagcgagccgtgcgcgctctgcagcctgcacagcatcggcaagatcggcggcgcgcagaaccgctcctacagcaagctgctgtgcggcctgctggccgagcgcctgcgcatcagcccggacagggtctacatcaactattacgacatgaacgcggccaatgtgggctggaacaactccaccttcgcctaagagccgcagggacccacgctgtctgcgctggctccacccgggaacccgccgcacgctgtgttctaggcccgcccaccccaaccttctggtggggagaaataaacggtttagagact

SEQ ID NO: 23 - DNA, 2586 bp, Homo Sapiens
agcgcgcaggcgcggccggattccgggcagtgacgcgacggcgggccgcgcggcgcatttccgcctctggcgaatggctcgtctgtagtgcacgccgcgggcccagctgcgaccccggccccgcccccgggaccccggccatggacgaactgttccccctcatcttcccggcagagccagcccaggcctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagaagagcagcgtggggactacgacctgaatgctgtgcggctctgcttccaggtgacagtgcgggacccatcaggcaggcccctccgcctgccgcctgtcctttctcatcccatctttgacaatcgtgcccccaacactgccgagctcaagatctgccgagtgaaccgaaactctggcagctgcctcggtggggatgagatcttcctactgtgtgacaaggtgcagaaagaggacattgaggtgtatttcacgggaccaggctgggaggcccgaggctccttttcgcaagctgatgtgcaccgacaagtggccattgtgttccggacccctccctacgcagaccccagcctgcaggctcctgtgcgtgtctccatgcagctgcggcggccttccgaccgggagctcagtgagcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaagggggtgacgcctgccctccccagagcactgggttgcaggggattgaagccctccaaaagcacttacggattctggtggggtgtgttccaactgcccccaactttgtggatgtcttccttggaggggggagccatattttattcttttattgtcagtatctgtatctctctctctttttggaggtgcttaagcagaagcattaacttctctggaaaggggggagctggggaaactcaaacttttcccctgtcctgatggtcagctcccttctctgtagggaactctggggtcccccatccccatcctccagcttctggtactctcctagagacagaagcaggctggaggtaaggcctttgagcccacaaagccttatcaagtgtcttccatcatggattcattacagcttaatcaaaataacgccccagataccagcccctgtatggcactggcattgtccctgtgcctaacaccagcgtttgaggggctggccttcctgccctacagaggtctctgccggctctttccttgctcaaccatggctgaaggaaaccagtgcaacagcactggctctctccaggatccagaaggggtttggtctgggacttccttgctctccctcttctcaagtgccttaatagtagggtaagttgttaagagtgggggagagcaggctggcagctctccagtcaggaggcatagtttttactgaacaatcaaagcacttggactcttgctctttctactctgaactaataaatctgttgccaagctggctagaaaaaaaaaaaaaaaaaa

SEQ ID NO: 24 - DNA, 2595 bp, Homo Sapiens
agcgcgcaggcgcggccggattccgggcagtgacgcgacggcgggccgcgcggcgcatttccgcctctggcgaatggctcgtctgtagtgcacgccgcgggcccagctgcgaccccggccccgcccccgggaccccggccatggacgaactgttccccctcatcttcccggcagagccagcccaggcctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagttcctatagaagagcagcgtggggactacgacctgaatgctgtgcggctctgcttccaggtgacagtgcgggacccatcaggcaggcccctccgcctgccgcctgtcctttctcatcccatctttgacaatcgtgcccccaacactgccgagctcaagatctgccgagtgaaccgaaactctggcagctgcctcggtggggatgagatcttcctactgtgtgacaaggtgcagaaagaggacattgaggtgtatttcacgggaccaggctgggaggcccgaggctccttttcgcaagctgatgtgcaccgacaagtggccattgtgttccggacccctccctacgcagaccccagcctgcaggctcctgtgcgtgtctccatgcagctgcggcggccttccgaccgggagctcagtgagcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaagggggtgacgcctgccctccccagagcactgggttgcaggggattgaagccctccaaaagcacttacggattctggtggggtgtgttccaactgcccccaactttgtggatgtcttccttggaggggggagccatattttattcttttattgtcagtatctgtatctctctctctttttggaggtgcttaagcagaagcattaacttctctggaaaggggggagctggggaaactcaaacttttcccctgtcctgatggtcagctcccttctctgtagggaactctggggtcccccatccccatcctccagcttctggtactctcctagagacagaagcaggctggaggtaaggcctttgagcccacaaagccttatcaagtgtcttccatcatggattcattacagcttaatcaaaataacgccccagataccagcccctgtatggcactggcattgtccctgtgcctaacaccagcgtttgaggggctggccttcctgccctacagaggtctctgccggctctttccttgctcaaccatggctgaaggaaaccagtgcaacagcactggctctctccaggatccagaaggggtttggtctgggacttccttgctctccctcttctcaagtgccttaatagtagggtaagttgttaagagtgggggagagcaggctggcagctctccagtcaggaggcatagtttttactgaacaatcaaagcacttggactcttgctctttctactctgaactaataaatctgttgccaagctggctagaaaaaaaaaaaaaaaaaa

SEQ ID NO: 25 - DNA, 4090 bp, Homo Sapiens
gtgagagagtgagcgagacagaaagagagagaagtgcaccagcgagccggggcaggaagaggaggtttcgccaccggagcggcccggcgacgcgctgacagcttcccctgcccttcccgtcggtcgggccgccagccgccgcagccctcggcctgcacgcagccaccggccccgctcccggagcccagcgccgccgaggccgcagccgcccggccagtaaggcggcgccgccgcccggccaccgcgcgccctgcgcttccctccgcccgcgctgcggccatggcgcggcgctgactggcctggcccggccccgccgcgctcccgctcgccccgacccgcactcgggcccgcccgggctccggcctgccgccgcctcttccttctccagccggcaggcccgcgccgcttaggagggagagcccacccgcgccaggaggccgaacgcggactcgccacccggcttcagaatggcagaagatgatccatatttgggaaggcctgaacaaatgtttcatttggatccttctttgactcatacaatatttaatccagaagtatttcaaccacagatggcactgccaacagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgctggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaatttagcctgctgacaatttcccacaccgtgtaaaccaaagccctaaaattccactgcgttgtccacaagacagaagctgaagtgcatccaaaggtgctcagagagccggcccgcctgaatcattctcgatttaactcgagaccttttcaacttggcttcctttcttggttcataaatgaattttagtttggttcacttacagatagtatctagcaatcacaacactggctgagcggatgcatctggggatgaggttgcttactaagctttgccagctgctgctggatcacagctgctttctgttgtcattgctgttgtccctctgctacgttcctattgtcattaaaggtatcacggtcgccacctggcattccttctgaccacagcatcattttgcattcaaattaagggttaagaaaagagatattttaaaatgagagtcacttgatgtgccattttaaaaaaaaaggcatattgctttttctaatgtggttatttctctgatttgcaaaaaaaaaaaaaaaaaaaatacttgtcaatatttaaacatggttacaatcattgctgaaaatggtattttcccccttttctgcattttgctattgtaaatatgttttttagatcaaatactttaaaggaaaaaatgttggatttataaatgctattttttattttacttttataataaaaggaaaagcaaattgatgacctcaaaaaaaaa

SEQ ID NO: 26 - DNA, 4093 bp, Homo Sapiens
gtgagagagtgagcgagacagaaagagagagaagtgcaccagcgagccggggcaggaagaggaggtttcgccaccggagcggcccggcgacgcgctgacagcttcccctgcccttcccgtcggtcgggccgccagccgccgcagccctcggcctgcacgcagccaccggccccgctcccggagcccagcgccgccgaggccgcagccgcccggccagtaaggcggcgccgccgcccggccaccgcgcgccctgcgcttccctccgcccgcgctgcggccatggcgcggcgctgactggcctggcccggccccgccgcgctcccgctcgccccgacccgcactcgggcccgcccgggctccggcctgccgccgcctcttccttctccagccggcaggcccgcgccgcttaggagggagagcccacccgcgccaggaggccgaacgcggactcgccacccggcttcagaatggcagaagatgatccatatttgggaaggcctgaacaaatgtttcatttggatccttctttgactcatacaatatttaatccagaagtatttcaaccacagatggcactgccaacagcagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgctggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaatttagcctgctgacaatttcccacaccgtgtaaaccaaagccctaaaattccactgcgttgtccacaagacagaagctgaagtgcatccaaaggtgctcagagagccggcccgcctgaatcattctcgatttaactcgagaccttttcaacttggcttcctttcttggttcataaatgaattttagtttggttcacttacagatagtatctagcaatcacaacactggctgagcggatgcatctggggatgaggttgcttactaagctttgccagctgctgctggatcacagctgctttctgttgtcattgctgttgtccctctgctacgttcctattgtcattaaaggtatcacggtcgccacctggcattccttctgaccacagcatcattttgcattcaaattaagggttaagaaaagagatattttaaaatgagagtcacttgatgtgccattttaaaaaaaaaggcatattgctttttctaatgtggttatttctctgatttgcaaaaaaaaaaaaaaaaaaaatacttgtcaatatttaaacatggttacaatcattgctgaaaatggtattttcccccttttctgcattttgctattgtaaatatgttttttagatcaaatactttaaaggaaaaaatgttggatttataaatgctattttttattttacttttataataaaaggaaaagcaaattgatgacctcaaaaaaaaa

SEQ ID NO: 27 - DNA, 1579 bp, Homo Sapiens
agcccacagcagtccgtgccgccgtcccgcccgccagcgccccagcgaggaagcagcgcgcagcccgcggcccagcgcacccgcagcagcgcccgcagctcgtccgcgccatgttccaggcggccgagcgcccccaggagtgggccatggagggcccccgcgacgggctgaagaaggagcggctactggacgaccgccacgacagcggcctggactccatgaaagacgaggagtacgagcagatggtcaaggagctgcaggagatccgcctcgagccgcaggaggtgccgcgcggctcggagccctggaagcagcagctcaccgaggacggggactcgttcctgcacttggccatcatccatgaagaaaaggcactgaccatggaagtgatccgccaggtgaagggagacctggccttcctcaacttccagaacaacctgcagcagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgttatgagcgcaaaggggctgaaagaacatggacttgtatatttgtacaaaaaaaaagttttatttttctaaaaaaagaaaaaagaagaaaaaatttaaagggtgtacttatatccacactgcacactgcctggcccaaaacgtcttattgtggtaggatcagccctcattttgttgcttttgtgaactttttgtaggggacgagaaagatcattgaaattctgagaaaacttcttttaaacctcacctttgtggggtttttggagaaggttatcaaaaatttcatggaaggaccacattttatatttattgtgcttcgagtgactgaccccagtggtatcctgtgacatgtaacagccaggagtgttaagcgttcagtgatgtggggtgaaaagttactacctgtcaaggtttgtgttaccctcctgtaaatggtgtacataatgtattgttggtaattattttggtacttttatgatgtatatttattaaacagatttttacaaatgaaaaaaaaaaaaa

SEQ ID NO: 28 - DNA, 2213 bp, Homo Sapiens
ccggcaaagcccagctacaggcgggcgactgcggggggcccctgaggcggcgggggccatggctggggtcgcgtgcttgggaaaagctgccgacgcagatgaatggtgcgacagcggcctgggctccctgggtccggacgcagcggcccccggaggacctgggttgggcgcggagttgggcccggggctgtcgtgggctcccctcgtcttcggctacgtcactgaggatggggacacgcttctcggccggcactgagtacatggacctgcagaatgacctaggccagacagccctgcacctggcagccatcctgggggagacatccacggtggagaagctgtacgcagcaggcgccgggctgtgtgtggcggagcgtaggggccacacggcgctgcacctggcctgccgtgtgggggcacacgcctgtgcccgtgccctgcttcagccccgcccccggcgccccagggaagcccccgacacctacctcgctcagggccctgaccgtactcccgacaccaaccatacccctgtcgccttgtaccccgattccgacttggagaaggaagaagaggagagtgaggaggactggaagctgcagctggaggctgaaaactacgagggccacaccccactccacgtggccgttatccacaaagatgtggagatggtccggctgctccgagatgctggagctgaccttgacaaaccggagcccacgtgcggccggagcccccttcatttggcagtggaggcccaggcagccgatgtgctggagcttctcctgagggcaggcgcgaaccctgctgcccgcatgtacggtggccgcaccccactcggcagtgccatgctccggcccaaccccatcctcgcccgcctcctccgtgcacacggagcccctgagcccgagggcgaggacgagaaatccggcccctgcagcagcagtagcgacagcgacagcggagacgagggcgtgagtcaggaggagagacagggcagcccagctggggggtcaggatagaccggcaggcaagaagcccaagaagataattaggcaccgaccttgggctgctgttagagaactcaggcagcagcgccagtgacacggggcactagtcaggagagacctggacaggggtggtgggaagagcttgggcagaagtggctgaaaaactaaggcagtggcaaaggtagaactcaggcaggggtggagaaaaggcgttggtcgcagtgattggtgaacacagcgggggtgggtggtagcgctgggggtgattttaggcagcaagaattggagaactcacactgcgaaaagaaaaccttgggtggcagtgatttgaacaccggcagtgctggggcaggacccgagccagcggtggggagagatatagtcagagaacccagcaatacagatccgtccttgggcaaggcgcggtgctggatgatgggtgcggaggaatttgggtaaaggcagagggaaggggtggaggagggccagctcagttgccgaaactctggagtggcggctggctagaaattggtctgtagaaatgaccttgaaaatggagttctggccaggtgcggtggctcacgcctgtaatcccagcactttgggaggccgaggcaggcagatcacgaggtcaggagttcgagaccagcctggccaacatggcaagaccctgtctctactaaaaatacaaaaattagctgggcgtggtggcgcatgcctataatcccagctacttgggaggctgaggtaggagaattgcttgaacctgggaggtggaggttgcagtgaacctagatcacgccactgcattccagcctgggcaacagagcgagactccatcttaaaaaaaaaaaaaaaaaaaagaaagaaaagaaaatggagttctgagagctaggaaactgggaggttgagccaggagcaggggaacttgggctttgaggcgagacctaattcttagggtgctctatgaggacatgggtggtgggcaaagtgacagccgcctgagcccctctgcctggtcccctttgcccccaggatgaatacgacgacattgtggttcacagcagccgcagccaaacccggctgcctcccaccccagcctcaaaacctcttcctgacgacccccgccccgtgtgatttgtttcattgttaatataatttccagtttaataaacaaaaccctagttctgacaaccagaaaaaaaa

SEQ ID NO: 29 - DNA, 1198 bp, Homo Sapiens
ccggcaaagcccagctacaggcgggcgactgcggggggcccctgaggcggcgggggccatggctggggtcgcgtgcttgggaaaagctgccgacgcagatgaatggtgcgacagcggcctgggctccctgggtccggacgcagcggcccccggaggacctgggttgggcgcggagttgggcccggggctgtcgtgggctcccctcgtcttcggctacgtcactgaggatggggacacggcactgcacttggctgtgattcatcagcatgaacccttcctggattttcttctaggcttctcggccggcactgagtacatggacctgcagaatgacctaggccagacagccctgcacctggcagccatcctgggggagacatccacggtggagaagctgtacgcagcaggcgccgggctgtgtgtggcggagcgtaggggccacacggcgctgcacctggcctgccgtgtgggggcacacgcctgtgcccgtgccctgcttcagccccgcccccggcgccccagggaagcccccgacacctacctcgctcagggccctgaccgtactcccgacaccaaccatacccctgtcgccttgtaccccgattccgacttggagaaggaagaagaggagagtgaggaggactggaagctgcagctggaggctgaaaactacgagggccacaccccactccacgtggccgttatccacaaagatgtggagatggtccggctgctccgagatgctggagctgaccttgacaaaccggagcccacgtgcggccggagcccccttcatttggcagtggaggcccaggcagccgatgtgctggagcttctcctgagggcaggcgcgaaccctgctgcccgcatgtacggtggccgcaccccactcggcagtgccatgctccggcccaaccccatcctcgcccgcctcctccgtgcacacggagcccctgagcccgagggcgaggacgagaaatccggcccctgcagcagcagtagcgacagcgacagcggagacgagggcgatgaatacgacgacattgtggttcacagcagccgcagccaaacccggctgcctcccaccccagcctcaaaacctcttcctgacgacccccgccccgtgtgatttgtttcattgttaatataatttccagtttaataaacaaaaccctagttctgacaaccagaaaaaaaa

SEQ ID NO: 30 - protein, 224 aa, Homo Sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 31 - protein, 99 aa, Homo Sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 32 - protein, 707 aa, Homo Sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 33 - protein, 233 aa, Homo Sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 34 - protein, 212 aa, Homo Sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 35 - protein, 115 aa, Homo Sapiens
MPMFIVNTNVPRASVPDGFLSELTQQLAQATGKPPQYIAVHVVPDQLMAFGGSSEPCALCSLHSIGKIGGAQNRSYSKLLCGLLAERLRISPDRVYINYYDMNAANVGWNNSTFA

SEQ ID NO: 36 - protein, 548 aa, Homo Sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 37 - protein, 551 aa, Homo Sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 38 - protein, 968 aa, Homo Sapiens
MAEDDPYLGRPEQMFHLDPSLTHTIFNPEVFQPQMALPTDGPYLQILEQPKQRGFRFRYVCEGPSHGGLPGASSEKNKKSYPQVKICNYVGPAKVIVQLVTNGKNIHLHAHSLVGKHCEDGICTVTAGPKDMVVGFANLGILHVTKKKVFETLEARMTEACIRGYNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPEIKDKEEVQRKRQKLMPNFSDSFGGGSGAGAGGGGMFGSGGGGGGTGSTGPGYSFPHYGFPTYGGITFHPGTTKSNAGMKHGTMDTESKKDPEGCDKSDDKNTVNLFGKVIETTEQDQEPSEATVGNGEVTLTYATGTKEESAGVQDNLFLEKAMQLAKRHANALFDYAVTGDVKMLLAVQRHLTAVQDENGDSVLHLAIIHLHSQLVRDLLEVTSGLISDDIINMRNDLYQTPLHLAVITKQEDVVEDLLRAGADLSLLDRLGNSVLHLAAKEGHDKVLSILLKHKKAALLLDHPNGDGLNAIHLAMMSNSLPCLLLLVAAGADVNAQEQKSGRTALHLAVEHDNISLAGCLLLEGDAHVDSTTYDGTTPLHIAAGRGSTRLAALLKAAGADPLVENFEPLYDLDDSWENAGEDEGVVPGTTPLDMATSWQVFDILNGKPYEPEFTSDDLLAQGDMKQLAEDVKLQLYKLLEIPDPDKNWATLAQKLGLGILNNAFRLSPAPSKTLMDNYEVSGGTVRELVEALRQMGYTEAIEVIQAASSPVKTTSQAHSLPLSPASTRQQIDELRDSDSVCDSGVETSFRKLSFTESLTSGASLLTLNKMPHDYGQEGPLEGKI

SEQ ID NO: 39 - protein, 969 aa, Homo Sapiens
MAEDDPYLGRPEQMFHLDPSLTHTIFNPEVFQPQMALPTADGPYLQILEQPKQRGFRFRYVCEGPSHGGLPGASSEKNKKSYPQVKICNYVGPAKVIVQLVTNGKNIHLHAHSLVGKHCEDGICTVTAGPKDMVVGFANLGILHVTKKKVFETLEARMTEACIRGYNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPEIKDKEEVQRKRQKLMPNFSDSFGGGSGAGAGGGGMFGSGGGGGGTGSTGPGYSFPHYGFPTYGGITFHPGTTKSNAGMKHGTMDTESKKDPEGCDKSDDKNTVNLFGKVIETTEQDQEPSEATVGNGEVTLTYATGTKEESAGVQDNLFLEKAMQLAKRHANALFDYAVTGDVKMLLAVQRHLTAVQDENGDSVLHLAIIHLHSQLVRDLLEVTSGLISDDIINMRNDLYQTPLHLAVITKQEDVVEDLLRAGADLSLLDRLGNSVLHLAAKEGHDKVLSILLKHKKAALLLDHPNGDGLNAIHLAMMSNSLPCLLLLVAAGADVNAQEQKSGRTALHLAVEHDNISLAGCLLLEGDAHVDSTTYDGTTPLHIAAGRGSTRLAALLKAAGADPLVENFEPLYDLDDSWENAGEDEGVVPGTTPLDMATSWQVFDILNGKPYEPEFTSDDLLAQGDMKQLAEDVKLQLYKLLEIPDPDKNWATLAQKLGLGILNNAFRLSPAPSKTLMDNYEVSGGTVRELVEALRQMGYTEAIEVIQAASSPVKTTSQAHSLPLSPASTRQQIDELRDSDSVCDSGVETSFRKLSFTESLTSGASLLTLNKMPHDYGQEGPLEGKI

SEQ ID NO: 40 - protein, 317 aa, Homo Sapiens
MFQAAERPQEWAMEGPRDGLKKERLLDDRHDSGLDSMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

SEQ ID NO: 41 - protein, 306 aa, Homo Sapiens
MNGATAAWAPWVRTQRPPEDLGWARSWARGCRGLPSSSATSLRMGTRFSAGTEYMDLQNDLGQTALHLAAILGETSTVEKLYAAGAGLCVAERRGHTALHLACRVGAHACARALLQPRPRRPREAPDTYLAQGPDRTPDTNHTPVALYPDSDLEKEEEESEEDWKLQLEAENYEGHTPLHVAVIHKDVEMVRLLRDAGADLDKPEPTCGRSPLHLAVEAQAADVLELLLRAGANPAARMYGGRTPLGSAMLRPNPILARLLRAHGAPEPEGEDEKSGPCSSSSDSDSGDEGVSQEERQGSPAGGSG

SEQ ID NO: 42 - protein, 356 aa, Homo Sapiens
MAGVACLGKAADADEWCDSGLGSLGPDAAAPGGPGLGAELGPGLSWAPLVFGYVTEDGDTALHLAVIHQHEPFLDFLLGFSAGTEYMDLQNDLGQTALHLAAILGETSTVEKLYAAGAGLCVAERRGHTALHLACRVGAHACARALLQPRPRRPREAPDTYLAQGPDRTPDTNHTPVALYPDSDLEKEEEESEEDWKLQLEAENYEGHTPLHVAVIHKDVEMVRLLRDAGADLDKPEPTCGRSPLHLAVEAQAADVLELLLRAGANPAARMYGGRTPLGSAMLRPNPILARLLRAHGAPEPEGEDEKSGPCSSSSDSDSGDEGDEYDDIVVHSSRSQTRLPPTPASKPLPDDPRPV

(19) United States
(12) Patent Application Publication
(10) Pub. No.: US 2010/0256196 A1
(43) Pub. Date: Oct. 7, 2010

(54) Biomarkers for Atherosclerosis

(75) Inventors: Andreas Pfuetzner, Mainz (DE); Thomas Forst, (US); Alexander Weise, (US)

Correspondence Address: MORGAN, LEWIS & BOCKIUS, LLP (SF), ONE MARKET SPEAR STREET TOWER, SAN FRANCISCO, CA, 94105, US

(73) Assignee: IKFE, Mainz (DE)

(21) Appl. No.: 12/631,707

(22) Filed: Dec. 4, 2009

Related U.S. Application Data
(60) Provisional application No. 61/119,982, filed on Dec. 4, 2008.

(51) Int. Cl. A61K 31/4439; C12Q 1/68; A61P 9/10

(52) U.S. Cl. 514/342; 435/6

(57) ABSTRACT

The invention provides compositions and methods for defining the state of atherosclerotic degeneration processes for the purposes of detection, severity assessment, monitoring and treatment. The states of atherosclerotic degeneration processes are identified by means of a biomarker panel particularly suited for detecting atherosclerotic degeneration processes. The simultaneous use of multiple markers with independent classification power will increase the performance of the panel in identifying atherosclerosis compared to other panels.

### CROSS-REFERENCE TO RELATED APPLICATIONS

This application claims under 35 USC 119(e) the benefit of U.S. Application 61/119,982, filed on Dec. 4, 2008, which is incorporated by reference in its entirety.

### TECHNICAL FIELD

The invention provides compositions and methods for defining the state of atherosclerotic degeneration processes for the purposes of detection, severity assessment, monitoring and treatment. The states of atherosclerotic degeneration processes are identified by means of a biomarker panel particularly suited for detecting atherosclerotic degeneration processes. The simultaneous use of multiple markers with independent classification power will increase the performance of the panel in identifying atherosclerosis compared to other panels.

### BACKGROUND

Obesity has been demonstrated to be associated with metabolic syndrome and cardiovascular disease, including severe complications, like acute coronary syndrome, myocardial infarction and stroke (1, 2) (see Appendix for full reference citations). An increase in body weight is usually accompanied by an increase in oxidative stress (3) and an elevation in the tissue expression and plasma levels of proinflammatory cytokines, such as tumor necrosis factor-α (TNFα) (4), interleukin-6 (IL-6) (5, 6), plasminogen activator-inhibitor-1 (PAI-1) (7) and others (8). This protein expression profile indicates the prevalence of a chronic systemic inflammation, and differentiating pre-adipocytes deriving from mesenchymal stem cells especially in the visceral lipid tissue are considered to be a major source for these cytokines and proteins (9). It is believed that the crosstalk between the pre-adipocytes and other tissues contributes to a general up-regulation of the immune system, including an activation of circulating monocytes and macrophages, resulting in an increased risk for atherosclerosis and vascular disease (10, 11).

It has been demonstrated by Ghanim and coworkers that circulating mononuclear cells in obese patients are in a proinflammatory state with an increase in intranuclear NF-κB binding, a decrease in IκB-β and an increase in the transcription of proinflammatory genes regulated by NF-κB, including migration inhibitory factor (MIF), IL-6, TNFα, and matrix metalloproteinase 9 (MMP-9) (12). The same group was able to demonstrate that an increased plasma concentration of MIF and an increased transcription of MIF mRNA in mononuclear cells, which was related to the body-mass index and hsCRP concentrations, could be reduced by a six week treatment with metformin in eight non-diabetic patients with obesity. The authors concluded that metformin might have beneficial effects on cardiovascular mortality in patients with type 2 diabetes (13), which is in part confirmed by the few currently existing larger outcome trials on this topic (14, 15). The same group also showed that the insulin sensitizing drug troglitazone was able to suppress NF-κB activity and stimulate IκB in non-diabetic obese patients, which gave evidence for an anti-inflammatory effect of this drug (16). Troglitazone was, however, taken from the market because of hepatotoxiciy (17).

It has been shown in randomized prospective trials that treatment with pioglitazone, another agonist to the peroxisome proliferators-activated receptor γ, may improve clinical and laboratory surrogate markers for atherosclerosis and cardiovascular risk, like intima-media thickness, hsCRP, or MMP-9 independent from glycemic control (18-20), and that it may even improve macrovascular outcome in type 2 diabetic patients when used in secondary prevention (21-23). The anti-inflammatory and anti-thrombotic effects of thiazolidinediones occur very rapidly and significantly earlier as compared to the metabolic and glycemic effects of these drugs (24, 25).

Current methods for assessing atherosclerosis in a subject include measuring indices/scores derived from general information, resulting in a % risk over a substantial time period. Other measures include clinical measures, e.g. intima-media-thickness determination via ultrasound, which only provides a static measure. There is no acute dynamic measurement currently used in clinical practice.

The current standard of care for subjects identified as being at risk for having atherosclerosis includes changes in life style (e.g., no smoking, more exercise etc.) and the prescription of drugs reducing LDL cholesterol (statins). Hypertension significantly raises the rate of cardiovascular events, and therefore is reduced by means of complex treatment guidelines. Potential insulin resistance driven risk is neither detected nor treated.

### SUMMARY OF INVENTION

In one aspect, the invention provides a kit comprising: (a) a first solid support comprising: (i) a capture binding ligand selective for hsCRP; and (b) a second solid support comprising: (i) a capture probe selective for MCP-1 nucleic acid; (ii) a capture probe selective for MMP-9 nucleic acid; and (iii) a capture probe selective for TNFα nucleic acid.

In one embodiment, the capture binding ligand comprises an antibody.

In one embodiment, the kit further comprises (a) a soluble capture ligand selective for hsCRP; wherein the soluble capture ligand comprises a detectable label.

In one embodiment, the kit further comprises: (a) a label probe selective for MCP-1 nucleic acid; (b) a label probe selective for MMP-9 nucleic acid; and (c) a label probe selective for TNFα nucleic acid; wherein each of the label probes comprises a detectable label.

In one embodiment, the kit further comprises: (a) a primer selective for MCP-1 nucleic acid; (b) a primer selective for MMP-9 nucleic acid; and (c) a primer selective for TNFα nucleic acid; wherein each of the primers optionally comprises a detectable label.

In one embodiment, the detectable label is a fluorophore.

In one embodiment, the detectable label comprises biotin.

In one embodiment, the kit further comprises a horseradish peroxidase conjugate.

In one embodiment, the kit further comprises a precipitating agent.

In one aspect, the invention provides a method of assaying a sample comprising (a) taking a measurement of the concentrations of hsCRP, MCP-1 nucleic acid, MMP-9 nucleic acid and TNFα nucleic acid in the sample.

In one embodiment, the sample is derived from a subject.

In one aspect, the invention provides a method of treating atherosclerosis in a subject comprising (a) measuring the concentrations of hsCRP, MCP-1 nucleic acid, MMP-9 nucleic acid and TNFα nucleic acid in a first sample from the subject; and (b) effecting a first therapy with respect to the subject.

In one embodiment, the concentration(s) of one, a combination or all of hsCRP, MCP-1 nucleic acid, MMP-9 nucleic acid and TNFα nucleic acid in a second sample from the subject decrease(s) after effecting the first therapy compared to corresponding concentration(s) in the first sample.

In one embodiment, the concentration(s) of one, a combination or all of MCP-1 nucleic acid, MMP-9 nucleic acid and TNFα nucleic acid in a second sample from the subject decrease(s) by at least about 15% compared to corresponding concentration(s) in the first sample.

In one embodiment, the concentration of hsCRP acid in a second sample from the subject decreases by about 10% to about 40% compared to the corresponding concentration in the first sample.

In one embodiment, the first therapy comprises administering a first disease-modulating drug to the subject.

In one aspect, the invention provides a method of assessing the efficacy of a first therapy on a subject comprising: (a) taking a first measurement of the concentrations of hsCRP, MCP-1 nucleic acid, MMP-9 nucleic acid and TNFα nucleic acid in a first sample from the subject; (b) effecting the first therapy on the subject; (c) taking a second measurement of the concentrations of hsCRP, MCP-1 nucleic acid, MMP-9 nucleic acid and TNFα nucleic acid in a second sample from the subject; and (d) making a comparison between the first and second measurements.

In one embodiment, the method further comprises (e) effecting a second therapy on the subject based on the comparison.

In one embodiment, effecting the first therapy comprises administering a first disease-modulating drug to the subject according to a first dosage regimen.

In one embodiment, effecting a second therapy comprises making a decision regarding the continued administration of the first disease-modulating drug.

In one embodiment, effecting a second therapy comprises administering a second disease-modulating drug to the subject.

In one embodiment, effecting a second therapy comprises administering a statin to the subject.

In one embodiment, effecting a second therapy comprises discontinuing the administration of the first disease-modulating drug.

In one embodiment, effecting a second therapy comprises repeating or maintaining the administration of the first disease-modulating drug.

In one embodiment, effecting a second therapy comprises administering the first disease-modulating drug according to an adjusted dosage regimen compared to the first dosage regimen.

In one embodiment, the adjusted dosage regimen depends on the degree of change in the concentration(s) of one, a combination or all of hsCRP, MCP-1 nucleic acid, MMP-9 nucleic acid and TNFα nucleic acid between the first and second measurement.

In one embodiment, if the concentration(s) of one, a combination or all of MCP-1 nucleic acid, MMP-9 nucleic acid and TNFα nucleic acid decrease(s) by at least about 15% between the first and second measurements, then effecting a second therapy comprises repeating or maintaining the administration of the first disease-modulating drug.

In one embodiment, if the concentration of hsCRP decreases by about 10% to about 40% between the first and second measurement, then effecting a second therapy comprises repeating or maintaining the administration of the first disease-modulating drug.

In one embodiment, if the concentration(s) of one, a combination or all of MCP-1 nucleic acid, MMP-9 nucleic acid and TNFα nucleic acid do(es) not decrease by at least about 15% between the first and second measurements, then effecting a second therapy comprises discontinuing the administration of the first disease-modulating drug.

In one embodiment, if the concentration of hsCRP does not decrease from about 10% to about 40% between the first and second measurements, then effecting a second therapy comprises discontinuing the administration of the first disease-modulating drug.

In one embodiment, the first disease-modulating drug is an insulin sensitizer.

In one embodiment, the insulin sensitizer is a glitazone.

In one embodiment, the glitazone is pioglitazone.

In one embodiment, the subject is experiencing atherosclerosis.

In one embodiment, a sample comprises blood.

In one embodiment, a sample is contacted with the first and/or second solid support of a kit disclosed herein.

In one aspect, the invention provides a method of acquiring data relating to sample comprising (a) taking a measurement of the concentrations of hsCRP, MCP-1 nucleic acid, MMP-9 nucleic acid and TNFα nucleic acid in the sample.

In one embodiment, the sample is derived from a subject, optionally wherein the subject is experiencing atherosclerosis.

In one embodiment, the sample comprises blood.

In one embodiment, the sample is contacted with the first and/or second solid support of any kit disclosed herein.

In one aspect, the invention provides use of a kit disclosed herein to determine a second therapy for a subject that has undergone a first therapy, wherein the subject is experiencing atherosclerosis.

In one aspect, the invention provides use of a kit disclosed herein to determine whether a subject belongs to a population that would benefit from a second therapy, wherein the subject has undergone a first therapy.

In one embodiment, the use comprises (a) contacting a first sample from the subject with the first and/or second solid support of the kit; (b) taking a first measurement of the concentrations of hsCRP, MCP-1 nucleic acid, MMP-9 nucleic acid and TNFα nucleic acid in the sample; (c) effecting a first therapy on the subject; (d) contacting a second sample from the subject with the first and/or second solid support of the kit; and (e) making a comparison of the first and second measurements.

In one embodiment, effecting the first therapy comprises administering a first disease-modulating drug to the subject according to a first dosage regimen.

In one embodiment, the second therapy comprises administering a second disease-modulating drug to the subject.

In one embodiment, the second therapy comprises administering a statin to the subject.

In one embodiment, the second therapy comprises discontinuing the administration of the first disease-modulating drug.

In one embodiment, the second therapy comprises repeating or maintaining the administration of the first disease-modulating drug.

In one embodiment, the second therapy comprises administering the first disease-modulating drug according to an adjusted dosage regimen compared to the first dosage regimen.

In one embodiment, the adjusted dosage regimen depends on the degree of change in the concentration(s) of one, a combination or all of hsCRP, MCP-1 nucleic acid, MMP-9 nucleic acid and TNFα nucleic acid between the first and second measurement.

In one embodiment, if the concentration(s) of one, a combination or all of MCP-1 nucleic acid, MMP-9 nucleic acid and TNFα nucleic acid decrease(s) by at least about 15% between the first and second measurements, then the second therapy comprises repeating or maintaining the administration of the first disease-modulating drug.

In one embodiment, if the concentration of hsCRP decreases by about 10% to about 40% between the first and second measurement, then the second therapy comprises repeating or maintaining the administration of the first disease-modulating drug.

In one embodiment, if the concentration(s) of one, a combination or all of MCP-1 nucleic acid, MMP-9 nucleic acid and TNFα nucleic acid do(es) not decrease by at least about 15% between the first and second measurements, then the second therapy comprises discontinuing the administration of the first disease-modulating drug.

In one embodiment, if the concentration of hsCRP does not decrease from about 10% to about 40% between the first and second measurements, then the second therapy comprises discontinuing the administration of the first disease-modulating drug.

In one embodiment, the first disease-modulating drug is an insulin sensitizer.

In one embodiment, the insulin sensitizer is a glitazone.

In one embodiment, the glitazone is pioglitazone.

In one embodiment, the subject is experiencing atherosclerosis.

In one embodiment, a sample comprises blood.

In any embodiment, a given biomarker panel can be replaced with another panel disclosed herein, such as a biomarker panel comprising or consisting of NFκB (e.g., p105 and relA), NFκB-modulated protein(s) (e.g., MMP9 and IL6) and NFκB inhibitor(s) (e.g., IκBα, IκBβ or IκBε).

### BRIEF DESCRIPTION OF THE DRAWINGS

FIG. 1 shows amplification curves of dilutions (1, 1:10, 1:100, 1:1000, 1:10000, 1:1000000) of cDNA (UL) amplified with primers for Rel-A on the LightCycler. cDNA samples were applied in triple replication (Repl.). For the resulting standard curve for Rel-A, sample crossing points were taken from amplification curves (mean value of triple replication). NC (negative control): water.

FIG. 2 shows changes in plasma concentrations of MMP-9, MCP-1, hsCRP, and glucose during the four weeks observation period.

FIG. 3 shows percent changes in proinflammatory mRNA expression markers from baseline to week 4.

FIGS. 4-16 shows sequences of biomarkers useful in the invention. As obvious to those of skill in the art, some sequences span more than one figure.

### DESCRIPTION OF EMBODIMENTS

The present invention is directed to the detection and/or quantification of a set of particular biomarkers (including but not limited to hsCRP, MCP-1, MMP-9 and TNFα) that allow for the detection of the presence and level of monocyte activation in a subject. The level of monocyte activation is an indicator of the acute nature of the ongoing process of atherosclerosis development. Thus, the present invention is also directed to determining the state of atherosclerosis in a subject. Measurement of the presence and quantity of the biomarkers provided herein allows for selection and monitoring of efficient risk-reducing treatment to avoid macrovascular complications. The effects of such treatment can also be monitored by the methods and compositions of the invention. A change in monocyte activation is an indicator of improvement or deterioration of a subject's vascular situation.

The assessment of a biomarker panel of the invention provides information regarding whether a patient has an ongoing acute risk of atherosclerosis development or impairment. The assessment of the biomarker panel in the context of a therapeutic intervention shows whether the chosen intervention improves a subject's overall cardiovascular risk, e.g. by reducing monocyte activation.

The identification of atherosclerotic risk can be a strong means to convince patients to comply with suggested lifestyle changes. The comparison of assay results before and after therapeutic intervention may demonstrate the immediate effect of the intervention, thus leading to higher patient compliance.

A large number of biomarkers are known for a variety of conditions; see US/2008/0057590, incorporated by reference in its entirety. However, the present invention is particularly directed to the use of a minimum number of biomarkers to provide a maximum amount of information concerning monocyte activation, and hence atherosclerotic degeneration processes, in a subject. The invention provides for the detection and quantification of levels of hsCRP, MCP-1, MMP-9 and TNFα, which in combination are useful as biomarkers for monocyte activation, partly because, as discussed below, each allows the assessment of a different aspect of monocyte activation. A panel of biomarkers consisting of hsCRP, MCP-1, MMP-9 and TNFα may be combined with measurements of other biomarkers and clinical parameters to assess monocyte activation. The invention also provides for the detection and quantification of levels of other biomarker panels, such as those comprising or consisting of NFκB (e.g., p105 and relA), NFκB-modulated protein(s) (e.g., MMP9 and IL6) and NFκB inhibitor(s) (e.g., IκBα or IκBβ) or combinations thereof for assessing monocyte activation.

Thus, the invention provides biological markers of atherosclerosis that in various combinations can be used in methods to monitor subjects that are undergoing therapies for atherosclerosis and to select or modify therapies or interventions for use in treating subjects with atherosclerosis.

### Biomarkers

Biomarkers may originate from epidemiological studies, animal studies, pathophysiological considerations and end-organ experiments. Ideally, a biomarker will have a high predictive value for a meaningful outcome measure, can be or is validated in appropriately designed prospective trials, reflects therapeutic success by corresponding changes in the surrogate marker results, and should be easy to assess in clinical practice.

The term “surrogate marker,” “biomolecular marker,” “biomarker” or “marker” (also sometimes referred to herein as a “target analyte,” “target species” or “target sequence”) refers to a molecule whose measurement provides information as to the state of a subject. In various exemplary embodiments, the biomarker is used to assess a pathological state. Measurements of the biomarker may be used alone or combined with other data obtained regarding a subject in order to determine the state of the subject. In one embodiment, the biomarker is “differentially present” in a sample taken from a subject of one phenotypic status (e.g., having a disease) as compared with another phenotypic status (e.g., not having the disease). In one embodiment, the biomarker is “differentially present” in a sample taken from a subject undergoing no therapy or one type of therapy as compared with another type of therapy. Alternatively, the biomarker may be “differentially present” even if there is no phenotypic difference, e.g. the biomarkers may allow the detection of asymptomatic risk. A biomarker may be determined to be “differentially present” in a variety of ways, for example, between different phenotypic statuses if the mean or median level or concentration (particularly the expression level of the associated mRNAs as described below) of the biomarker in the different groups is calculated to be statistically significant. Common tests for statistical significance include, among others, t-test, ANOVA, Kruskal-Wallis, Wilcoxon, Mann-Whitney and odds ratio.

As described herein, a biomarker may be, for example, a small molecule, an analyte or target analyte, a lipid (including glycolipids), a carbohydrate, a nucleic acid, a protein, any derivative thereof or a combination of these molecules, with proteins and nucleic acids finding particular use in the invention. As will be appreciated by those in the art, a large number of analytes may be detected using the present methods; basically, any biomarker for which a binding ligand, described below, may be made may be detected using the methods of the invention.

In various embodiments, the biomarkers used in the panels of the invention can be detected either as proteins or as nucleic acids (e.g. mRNA or cDNA transcripts) in any combination. In various embodiments, the protein form of a biomarker is measured. As will be appreciated by those in the art, protein assays may be done using standard techniques such as ELISA assays. In various embodiments, the nucleic acid form of a biomarker (e.g., the corresponding mRNA) is measured. In various exemplary embodiments, one or more biomarkers from a particular panel are measured using a protein assay and one or more biomarkers from the same panel are measured using a nucleic acid assay.

As will be appreciated by those in the art, there are a large number of possible proteinaceous target analytes and target species that may be detected using the present invention. The term “protein,” “polypeptide” or “oligopeptide” refers to at least two or more peptides or amino acids joined by one or more peptide bonds. A protein or an amino acid may be naturally or nonnaturally occurring and may be also be an analog, a derivative or a peptidomimetic structure. A protein can have a wild-type sequence, a variant of wild-type sequence or either of these containing one or more analogs or derivatized amino acids. A variant may contain one or more additions, deletions or substitutions of one or more peptides compared to wild-type or a different variant sequence. Examples of derivatized amino acids include, without limitation, those that have been modified by the attachment of labels (described below); acetylation; acylation; ADP-ribosylation; amidation; covalent attachment of flavin, a heme moiety, a nucleotide, a lipid or phosphatidylinositol; cross-linking; cyclization; disulfide bond formation; demethylation; esterification; formation of covalent crosslinks, cystine or pyroglutamate; formylation; gamma carboxylation; glycosylation; GPI anchor formation; hydroxylation; iodination; methylation; myristoylation; oxidation; proteolytic processing; phosphorylation; prenylation; racemization; selenoylation; sulfation; and ubiquitination. Such modifications are well-known to those of skill in the art and have been described in great detail in the scientific literature. Several particularly common modifications such as glycosylation, lipid attachment, sulfation, gamma-carboxylation, hydroxylation and ADP-ribosylation, for instance, are described in basic texts, such as Creighton, Proteins—Structure and Molecular Properties, 2d ed. (New York: W. H. Freeman and Company, 1993). Many detailed reviews are available on this subject, such as in Johnson, ed., Posttranslational Covalent Modification of Proteins (New York: Academic Press, 1983); Seifter et al., Meth. Enzymol., 1990, 182: 626-646; and Rattan et al., Ann. N.Y. Acad. Sci., 1992, 663: 48-62. As discussed below, when the protein is used as a binding ligand, it may be desirable to utilize protein analogs to retard degradation by sample contaminants.

In various exemplary embodiments, the biomarker is a nucleic acid. The term “nucleic acid”, “oligonucleotide” or “polynucleotide” herein means at least two nucleotides covalently linked together. A nucleic acid of the present invention will generally contain phosphodiester bonds, although in some cases, for example in the use of binding ligand probes, nucleic acid analogs are included that may have alternate backbones, comprising, for example, phosphoramide (Beaucage et al., Tetrahedron, 49(10): 1925 (1993) and references therein; Letsinger, J. Org. Chem. 35: 3800 (1970); Sprinzl et al., Eur. J. Biochem. 81:579 (1977); Letsinger et al., Nucl. Acids Res. 14: 3487 (1986); Sawai et al, Chem. Lett. 13(5): 805 (1984); Letsinger et al., J. Am. Chem. Soc. 110:4470 (1988); and Pauwels et al., Chemica Scripta 26:141 (1986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, (Oxford University Press, 1991), and peptide nucleic acid backbones and linkages (see Egholm, J. Am. Chem. Soc. 114: 1895 (1992); Meier et al., Chem. Int. Ed. Engl. 31: 1008 (1992); Nielsen, Nature, 365: 566 (1993); Carlsson et al., Nature, 380: 207 (1996), all of which are incorporated by reference). Other analog nucleic acids include those with positive backbones (Denpcy et al., Proc. Natl. Acad. Sci. USA 92: 6097 (1995)), non-ionic backbones (U.S. Pat. Nos. 5,386,023; 5,637,684; 5,602,240; 5,216,141 and 4,469,863; Kiedrowshi et al., Angew. Chem. Intl. Ed. English 30: 423 (1991); Letsinger et al., J. Am. Chem. Soc. 110: 4470 (1988); Letsinger et al., Nucleoside & Nucleotide 13: 1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4: 395 (1994); Jeffs et al., J. Biomolecular NMR 34: 17 (1994); and Horn et al., Tetrahedron Lett. 37: 743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev., 24: 169-176 (1995)). Several nucleic acid analogs are described in Rawls, C & E News, 35 (Jun. 2, 1997). All of these references are hereby expressly incorporated by reference. These modifications of the ribose-phosphate backbone may be done to increase the stability and half-life of such molecules in physiological environments. As will be appreciated by those in the art, all of these nucleic acid analogs may find use in the present invention. In addition, mixtures of naturally occurring nucleic acids and analogs can be made.

In various embodiments, variants of the sequences described herein, including proteins and nucleic acids based on e.g. splice variants, variants comprising a deletion, addition, substitution, fragment, preproprotein, processed preproprotein (e.g. without a signaling peptide), processed proprotein (e.g. resulting in an active form), nonhuman sequences and variant nonhuman sequences may be used as biomarkers. In some embodiments, the variant sequence has a homology compared to a parent sequence, such as a sequence described herein, of about a percentage selected from 30%, 40%, 50%, 60%, 70%, 80%, 90%, 91%, 92%, 93%, 94%, 95%, 96%, 97%, 98% and 99%.

It has been found that assays involving the measurement of hsCRP, MCP-1, MMP-9 and TNFα in various combinations have greater value in determining atherosclerotic degeneration processes than any of these biomarkers alone. Combinations of these biomarkers allow attainment of clinically useful sensitivity and specificity. Accordingly, measurements of a biomarker panel comprising or consisting of hsCRP, MCP-1, MMP-9 and TNFα in various combinations may be used to improve the sensitivity and/or specificity of a diagnostic test compared to a test involving any one of these biomarkers alone. Other biomarker panels useful for determining atherosclerotic degeneration processes include those comprising or consisting of NFκB (e.g., p105 and relA), NFκB-modulated protein(s) (e.g., MMP9 and IL6) and NFκB inhibitor(s) (e.g., IκBα or IκBβ).

High Sensitivity C-Reactive Protein (hsCRP)

In various embodiments, CRP (C-reactive protein) or hsCRP (high sensitivity C-reactive protein) is used as a biomarker. CRP is a member of the pentraxin family, comprising five noncovalently associated protomers arranged symmetrically around a central pore and has a molecular weight of 118,000 Da. (See generally, halal et al., Hypertension, 2004, 44: 6-11) CRP is a marker of inflammation that has been shown to predict incident myocardial infarction, stroke, peripheral arterial disease, and sudden cardiac death process. Ridker, Circulation, 2003, 107: 363-369. Various epidemiological studies involving individuals with no prior history of cardiovascular disease have shown that a single, non-fasting measure of CRP is a strong predictor of future vascular events. The predictive value of CRP has proven independent of major traditional risk factors, such as age, smoking, cholesterol levels, blood pressure and diabetes.

High-sensitivity CRP assays have been developed and are now widely available. (Roberts et al., Clinical Chemistry 2001, 47: 444-450.) In one embodiment, a biomarker, such as hsCRP, is measured by immune turbidometry.

In various embodiments, hsCRP is derived from a peptide sequence according to RefSeq Accession Record NP_{—}000558 or is derived from a nucleic acid sequence according to RefSeq Accession Record NM_{—}000567.

In exemplary embodiments, the protein form of hsCRP is measured. Accordingly, suitable capture binding ligands, as further discussed herein, for detection and/or quantification of hsCRP include, but are not limited to, antibodies that are selective for hsCRP. hsCRP antibodies are known and commercially available.

In response to a therapy, such as administration of a disease-modulating drug, as described below, the levels of hsCRP will decrease if the patient is responding to the therapy. In patients with chronic systemic inflammation, decreases occur from levels of about 3-10 mg/L to levels of about 2-3 mg/L, with changes of at least about 10-15% to about 30-40% being more determinative of a response. In some embodiments, a decrease of about 25% to about 30% from a baseline value indicates a response. In some instances, a change of at least about a percentage selected from 10%, 20%, 30%, 40%, 50%, 60%, 70%, 80% and 90% from a baseline value will occur. A change of about 10% to about 100% can also be observed. An hsCRP level after two weeks of treatment between 0 to 1 mg/L indicates a low remaining systemic inflammation and corresponding cardiovascular risk, 1 to 3 mg/L indicate a moderate remaining risk and 3 to 10 mg/L indicate a high remaining risk. Values above 10 mg/L may be caused by other unspecific inflammation (e.g. infections) and do not have a predictive value for cardiovascular risk. In some embodiments, decreases occur from levels of about 3-10 mg/L to levels of about 0-1 mg/L. In some embodiments, decreases occur from levels of about 2-3 mg/L to levels of about 0-1 mg/L.

As is more fully described below, it is also possible that the patient is responding to a therapy, such as an insulin sensitizer drug, as shown by changes in other biomarkers, but the levels of hsCRP are not changing in a significant way. This is specifically the case if other risk factors or diseases interfere with chronic systemic inflammation in regards to macrophage activation.

### NFκB-Modulated Proteins

In various embodiments, proteins modulated or regulated by NFκB is used as a biomarker. These proteins include, but are not limited to, plasma cytokines and proinflammatory mediators and markers. Examples of proteins modulated by NFκB include, but are not limited to, tumor necrosis factor α (TNFα), interleukin 6 (IL6), monocyte chemoattractant protein-1 (MCP-1), macrophage migration inhibitory factor (MIF) and matrix metallopeptidase 9 (MMP9).

### MCP-1

In various embodiments, monocyte chemoattractant protein-1 (MCP-1) is used as a biomarker. MCP-1, also known as chemokine (C—C motif) ligand 2 (CCL2), is an essential chemokine involved in monocyte traffic across endo- and epithelial barriers both in vitro and in vivo. MCP-1 is transcriptionally regulated by NFκB. The structure of MCP-1 is related to that of the CXC subfamily of cytokines, which are characterized by two cysteines separated by a single amino acid. MCP-1 displays chemotactic activity for monocytes and basophils but not for neutrophils or eosinophils, and has been found to bind to chemokine receptors CCR2 and CCR4. It has been implicated in the pathogenesis of diseases characterized by monocytic infiltrates, like psoriasis, rheumatoid arthritis and atherosclerosis.

In various embodiments, MCP-1 is derived from a peptide sequence according to RefSeq Accession Record NP_{—}002973 or is derived from a nucleic acid sequence according to RefSeq Accession Record NM_{—}002982.

In exemplary embodiments, the nucleic acid (e.g. mRNA) form of MCP-1 is measured. As is known in the art, a wide variety of methods for detecting mRNA are known, particularly on arrays. This includes the direct measurement of mRNA as well as treating the same with reverse transcriptase and measuring cDNA levels. Accordingly, suitable capture probes, as further discussed below, for the detection and/or quantification of MCP-1 mRNA include, but are not limited to, fragments of the complements of the mRNA sequences of MCP-1. That is, if the mRNA is to be directly detected, a complementary sequence will be used to bind the single stranded mRNA. In general, as for all the capture probes outlined herein, the probes generally are between about 5 and about 100 basepairs in length, with from about 6 to about 30, about 8 to about 28, and about 16 to about 26 being of particular use in some embodiments.

In response to a therapy, such as administration of a disease-modulating drug, as described below, the levels of MCP-1 will decrease if the patient is responding to the therapy. In some embodiments, this decrease is in the range of about 10% to about 20% from a baseline value. In some embodiments, the concentration of MCP-1 decreases at least about a percentage selected from 10%, 11%, 12%, 13%, 14%, 15%, 16%, 17%, 18%, 19% and 20% from a baseline value. In some embodiments, the concentration of MCP-1 decreases at least about 15% from a baseline value.

As is more fully described below, it is also possible that the patient is responding to a therapy, such as an insulin sensitizer drug, as shown by changes in other biomarkers, but the levels of MCP-1 are not changing in a significant way.

### MMP-9

In various embodiments, matrix metalloproteinase is used as a biomarker. Proteins of the matrix metalloproteinase (MMP) family are involved in the breakdown of extracellular matrix in normal physiological processes, such as embryonic development, reproduction, and tissue remodeling, as well as in disease processes, such as arthritis and metastasis. Most MMPs are secreted as inactive proproteins, which are activated when cleaved by extracellular proteinases. The enzyme encoded by this gene degrades type IV and V collagens. Studies suggest that the enzyme is involved in IL-8-induced mobilization of hematopoietic progenitor cells from bone marrow, and murine studies suggest a role in tumor-associated tissue remodeling.

In various embodiments, MMP-9 is derived from peptide sequence according to RefSeq Accession Record NP_{—}004985 or is derived from a nucleic acid sequence according to RefSeq Accession Record NM_{—}004994.

In exemplary embodiments, the nucleic acid (e.g. mRNA) form of MMP-9 is measured. As is known in the art, a wide variety of methods for detecting mRNA are known, particularly on arrays. This includes the direct measurement of mRNA as well as treating the same with reverse transcriptase and measuring the cDNA levels. Accordingly, suitable capture probes for the detection and/or quantification of MMP-9 mRNA include, but are not limited to, fragments of the complements of MMP-9 mRNA. That is, if the mRNA is to be directly detected, a complementary sequence will be used to bind the single stranded mRNA. In general, as for all the capture probes outlined herein, the probes generally are between about 5 and about 100 basepairs in length, with about 6 to about 30, about 8 to about 28, and about 16 to about 26 being of particular use in some embodiments.

In response to a therapy, such as administration of a disease-modulating drug, as described below, the levels of MMP-9 will decrease if the patient is responding to the therapy. In some embodiments, this decrease is in a range of about 25% to about 30% from a baseline value. In some embodiments, the concentration of MMP-9 decreases at least about a percentage selected from 10%, 11%, 12%, 13%, 14%, 15%, 16%, 17%, 18%, 19% and 20% from a baseline value. In some embodiments, the concentration of MMP-9 decreases at least about 15% from baseline value.

As is more fully described below, it is also possible that the patient is responding to a therapy, such as an insulin sensitizer drug, as shown by changes in other biomarkers, but the levels of MMP-9 are not changing in a significant way.

### TNFα

In various exemplary embodiments, tumor necrosis factor alpha (TNFα) is used as a biomarker. TNFα, also known simply as TNF, is a multifunctional proinflammatory cytokine that belongs to the TNF superfamily and is mainly secreted by macrophages. It can bind to, and thus functions through its receptors TNFRSF1A/TNFR1 and TNFRSF1B/TNFBR. TNFα is involved in the regulation of a wide spectrum of biological processes including cell proliferation, differentiation, apoptosis, lipid metabolism, and coagulation. TNFα has been implicated in a variety of diseases, including autoimmune diseases, insulin resistance, and cancer. Knockout studies in mice also suggested the neuroprotective function of this cytokine.

In various embodiments, TNFα is derived from a peptide sequence according to RefSeq Accession Record NP_{—}000585 or is derived from a nucleic acid sequence according to RefSeq Accession Record NM_{—}000594.

In exemplary embodiments, the mRNA form of TNFα is measured. As is known in the art, a wide variety of methods for detecting mRNA are known, particularly on arrays. This includes the direct measurement of mRNA as well as treating the same with reverse transcriptase and measuring the cDNA levels. Accordingly, suitable capture probes for the detection and/or quantification of TNFα mRNA include, but are not limited to, fragments of the complements of TNFα mRNA. That is, if the mRNA is to be directly detected, a complementary sequence will be used to bind the single stranded mRNA. In general, as for all the capture probes outlined herein, the probes generally are between about 5 and about 100 basepairs in length, with about 6 to about 30, about 8 to about 28, and about 16 to about 26 being of particular use in some embodiments.

In response to a therapy, such as administration of a disease-modulating drug, as described below, the levels of TNFα will decrease if the patient is responding to the therapy. In some embodiments, the concentration of TNFα decreases at least about a percentage selected from 10%, 11%, 12%, 13%, 14%, 15%, 16%, 17%, 18%, 19% and 20% from a baseline value. In some embodiments, the concentration of TNFα decreases at least about 15% from baseline value.

As is more fully described below, it is also possible that the patient is responding to a therapy, such as an insulin sensitizer drug, as shown by changes in other biomarkers, but the levels of TNFα are not changing in a significant way.

### IL6

In various embodiments, Interleukin-6 (interferon, beta 2) (IL6) is used as a biomarker. IL6 is an immunoregulatory cytokine that activates a cell surface signaling assembly composed of IL6, IL6RA (IL6R; MIM 147880), and the shared signaling receptor gp130 (IL6ST; MIM 600694) (Boulanger et al., 2003 [PubMed 12829785]).

In various embodiments, IL6 is derived from a peptide sequence according to RefSeq Accession Record NP_{—}000591 or is derived from a nucleic acid sequence according to RefSeq Accession Record NM_{—}000600.

In exemplary embodiments, the nucleic acid (e.g. mRNA) form of IL6 is measured. As is known in the art, a wide variety of methods for detecting mRNA are known, particularly on arrays. This includes the direct measurement of mRNA as well as treating the same with reverse transcriptase and measuring the cDNA levels. Accordingly, suitable capture probes for the detection and/or quantification of IL6 mRNA include, but are not limited to, fragments of the complements of IL6 mRNA. That is, if the mRNA is to be directly detected, a complementary sequence will be used to bind the single stranded mRNA. In general, as for all the capture probes outlined herein, the probes generally are between about 5 and about 100 basepairs in length, with about 6 to about 30, about 8 to about 28, and about 16 to about 26 being of particular use in some embodiments.

In response to a therapy, such as administration of a disease-modulating drug, as described below, the levels of IL6 will decrease if the patient is responding to the therapy. In some embodiments, the concentration of IL6 decreases at least about a percentage selected from 10%, 11%, 12%, 13%, 14%, 15%, 16%, 17%, 18%, 19% and 20% from a baseline value. In some embodiments, the concentration of IL6 decreases at least about 15% from a baseline value.

As is more fully described below, it is also possible that the patient is responding to a therapy, such as an insulin sensitizer drug, as shown by changes in other biomarkers, but the levels of IL6 are not changing in a significant way.

### MIF

In various exemplary embodiments, macrophage migration inhibitory factor (glycosylation-inhibiting factor) (MIF) is used as a biomarker. MIF is a lymphokine involved in cell-mediated immunity, immunoregulation, and inflammation, and plays a role in the regulation of macrophage function in host defense through the suppression of anti-inflammatory effects of glucocorticoids. MIF and JAB1 protein form a complex in the cytosol near the peripheral plasma membrane.

In various embodiments, MIF is derived from a peptide sequence according to RefSeq Accession Record NP_{—}002406 or is derived from a nucleic acid sequence according to RefSeq Accession Record NM_{—}002415.

In exemplary embodiments, the nucleic acid (e.g. mRNA) form of MIF is measured. As is known in the art, a wide variety of methods for detecting mRNA are known, particularly on arrays. This includes the direct measurement of mRNA as well as treating the same with reverse transcriptase and measuring the cDNA levels. Accordingly, suitable capture probes for the detection and/or quantification of MIF mRNA include, but are not limited to, fragments of the complements of MIF mRNA. That is, if the mRNA is to be directly detected, a complementary sequence will be used to bind the single stranded mRNA. In general, as for all the capture probes outlined herein, the probes generally are between about 5 and about 100 basepairs in length, with about 6 to about 30, about 8 to about 28, and about 16 to about 26 being of particular use in some embodiments.

In response to a therapy, such as administration of a disease-modulating drug, as described below, the levels of MIF will decrease if the patient is responding to the therapy. In some embodiments, the concentration of MIF decreases at least about a percentage selected from 10%, 11%, 12%, 13%, 14%, 15%, 16%, 17%, 18%, 19% and 20% from a baseline value. In some embodiments, the concentration of MIF decreases at least about 15% from baseline value.

As is more fully described below, it is also possible that the patient is responding to a therapy, such as an insulin sensitizer drug, as shown by changes in other biomarkers, but the levels of MIF are not changing in a significant way.

In some embodiments, two NFκB modulated proteins are used as biomarkers in a panel. In some instances, three NFκB modulated proteins are used in the biomarker panel, and in some instances four or more are used.

### NFκB

In various embodiments, nuclear factor κ-light-chain-enhancer of activated B cells (NFκB) is used as a biomarker. NFκB is a transcription factor involved in inflammation, autoimmune response, cell proliferation and apoptosis. NFκB functions by regulating gene expression of these processes. NFκB comprises homo- or heterodimers of different subunits belonging to the Rel/NFκB family of proteins. The Rel proteins include, but are not limited to, p50 (derived from p105), p52 (derived from p100), p65 (also called RelA), RelB and c-Rel. One prevalent form of NFκB is a heterodimer of RelA and either p50 or p52.

As used herein, the term “NFκB” may refer to a subunit of NFκB or any combination of subunits. In various exemplary embodiments, one or more NFκB nucleic acids (e.g. mRNA) is measured. In one embodiment RelA nucleic acid is measured. In one embodiment, RelA nucleic acid is derived from a sequence according to RefSeq Accession Record NM_{—}001145138 or NM_{—}021975. The term “NFκB” may also refer to a preprocessed or precursor form of a subunit, including proproteins and preproproteins. p105 is a 105-kDa precursor of the p50 subunit of NFκB. p50 is derived from the N-terminal portion of p105, which contains the Rel homology domain. Thus, in one embodiment, p105 nucleic acid is measured. In one embodiment, p105 nucleic acid is derived from a sequence according to RefSeq Accession Record NM_{—}001165412 or NM_{—}003998. In one embodiment, RelA protein (RefSeq Accession Record NP_{—}001138610 or NP_{—}068810) or p105 protein (RefSeq Accession Record NP_{—}001158884 or NP_{—}003989) is measured.

As is known in the art, a wide variety of methods for detecting mRNA are known, particularly on arrays. This includes the direct measurement of mRNA as well as treating the same with reverse transcriptase and measuring the cDNA levels. Accordingly, suitable capture probes for the detection and/or quantification of NFκB mRNA include, but are not limited to, fragments of the complements of the mRNA sequences of RelA and p105. That is, if the mRNA is to be directly detected, a complementary sequence will be used to bind the single stranded mRNA. In general, as for all the capture probes outlined herein, the probes generally are between about 5 and about 100 basepairs in length, with from about 6 to about 30, about 8 to about 28, and about 16 to about 26 being of particular use in some embodiments.

In response to a therapy, such as administration of a disease-modulating drug, as described below, the expression levels of NFκB as determined in relation to a housekeeping gene (e.g. β-actin) will decrease if the patient is responding to the therapy by about 25% to about 30%. In some embodiments, the concentration of NFκB decreases at least about a percentage selected from 10%, 11%, 12%, 13%, 14%, 15%, 16%, 17%, 18%, 19% and 20% from a baseline level. In some embodiments, the concentration of NFκB decreases at least about 15% from baseline level.

As is more fully described below, it is also possible that the patient is responding to a therapy, such as an insulin sensitizer drug, as shown by changes in other biomarkers, but the levels of NFκB are not changing in a significant way.

### NFκB Inhibitors

In various embodiments, one or more NFκB inhibitors are used as biomarkers in the panels described herein. In inactive form, NFκB is bound to regulatory proteins called inhibitors of KB (IκB), which include, but are not limited to, IκBα, IκBβ and IκBε. Expression of inhibitors of NFκB should therefore change in the opposite direction compared to NFκB in response to a therapy.

### IκBα

In various exemplary embodiments, IκBα is used as a biomarker. IκBα masks the nuclear localization signals (NLS) of NFκB, thus maintaining NFκB in an inactive state in the cytoplasm. Phosphorylation of serine residues on the IκB proteins by kinases, for example, IKBKA or IKBKB, marks them for destruction via the ubiquitination pathway, thereby allowing activation of NFκB.

In various embodiments, IκBα is derived from a peptide sequence according to RefSeq Accession Record NP_{—}065390 or is derived from a nucleic acid sequence according to RefSeq Accession Record NM_{—}020529.

In exemplary embodiments, the nucleic acid (e.g. mRNA) form of IκBα is measured. As is known in the art, a wide variety of methods for detecting mRNA are known, particularly on arrays. This includes the direct measurement of mRNA as well as treating the same with reverse transcriptase and measuring the cDNA levels. Accordingly, suitable capture probes for the detection and/or quantification of IκBα mRNA include, but are not limited to, fragments of the complements of IκBα mRNA. That is, if the mRNA is to be directly detected, a complementary sequence will be used to bind the single stranded mRNA. In general, as for all the capture probes outlined herein, the probes generally are between about 5 and about 100 basepairs in length, with about 6 to about 30, about 8 to about 28, and about 16 to about 26 being of particular use in some embodiments.

In response to a therapy, such as administration of a disease-modulating drug, as described below, the levels of IκBα will increase if the patient is responding to the therapy. In some embodiments, the concentration of IκBα increases at least about a percentage selected from 10%, 11%, 12%, 13%, 14%, 15%, 16%, 17%, 18%, 19% and 20% from a baseline level. In some embodiments, the concentration of IκBα increases at least about 15% from baseline level.

As is more fully described below, it is also possible that the patient is responding to a therapy, such as an insulin sensitizer drug, as shown by changes in other biomarkers, but the levels of IκBα are not changing in a significant way.

### IκBβ

In various exemplary embodiments, IκBβ is used as a biomarker.

In various embodiments, IκBβ is derived from a peptide sequence according to RefSeq Accession Record NP_{—}001001716 or NP_{—}002494 or is derived from a nucleic acid sequence according to RefSeq Accession Record NM_{—}001001716 or NM_{—}002503.

In exemplary embodiments, the nucleic acid (e.g. mRNA) form of IκBβ is measured. As is known in the art, a wide variety of methods for detecting mRNA are known, particularly on arrays. This includes the direct measurement of mRNA as well as treating the same with reverse transcriptase and measuring the cDNA levels. Accordingly, suitable capture probes for the detection and/or quantification of IκBβ mRNA include, but are not limited to, fragments of the complements of IκBβ mRNA. That is, if the mRNA is to be directly detected, a complementary sequence will be used to bind the single stranded mRNA. In general, as for all the capture probes outlined herein, the probes generally are between about 5 and about 100 basepairs in length, with about 6 to about 30, about 8 to about 28, and about 16 to about 26 being of particular use in some embodiments.

In response to a therapy, such as administration of a disease-modulating drug, as described below, the levels of IκBβ will increase if the patient is responding to the therapy. In some embodiments, the concentration of IκBβ increases at least about a percentage selected from 10%, 11%, 12%, 13%, 14%, 15%, 16%, 17%, 18%, 19% and 20% from a baseline level. In some embodiments, the concentration of IκBa increases at least about 15% from a baseline level.

As is more fully described below, it is also possible that the patient is responding to a therapy, such as an insulin sensitizer drug, as shown by changes in other biomarkers, but the levels of IκBβ are not changing in a significant way.

In some embodiments, two NFκB inhibitors are used as biomarkers. Suitable pairs include, but are not limited to, IκBα and IκBβ; IκBα and IκBε; and IκBβ and IκBε. In some instances, three NFκB inhibitors are used in the biomarker panel, and in some instances four or more.

### Biomarker Panels

Any combination of the biomarkers described herein can be used to assemble a biomarker panel, which is detected or measured as described herein. As is generally understood in the art, a combination may refer to an entire set or any subset or subcombination thereof. The term “biomarker panel,” “biomarker profile,” or “biomarker fingerprint” refers to a set of biomarkers. As used herein, these terms can also refer to any form of the biomarker that is measured. Thus, if MMP-9 is part of a biomarker panel, then either MMP-9 protein or MMP-9 mRNA, for example, could be considered to be part of the panel. While individual biomarkers are useful as diagnostics, it has been found that a combination of biomarkers can sometimes provide greater value in determining a particular status than single biomarkers alone. Specifically, the detection of a plurality of biomarkers in a sample can increase the sensitivity and/or specificity of the test. Thus, in various embodiments, a biomarker panel may include 2, 3, 4, 5, 6, 7, 8, 9, 10 or more types of biomarkers. In various exemplary embodiments, the biomarker panel consists of a minimum number of biomarkers to generate a maximum amount of information. Thus, in various embodiments, the biomarker panel consists of 2, 3, 4, 5, 6, 7, 8, 9 or 10 types of biomarkers. Where a biomarker panel “consists of” a set of biomarkers, no biomarkers other than those of the set are present.

The present invention provides a biomarker panel comprising or consisting of any combination of the biomarkers outlined herein.

In various exemplary embodiments, the biomarker panel comprises additional biomarkers. Such additional biomarkers may, for example, increase the specificity and/or sensitivity the test. For example, additional biomarkers may be those that are currently evaluated in the clinical laboratory and used in traditional global risk assessment algorithms, such as those from the San Antonio Heart Study, the Framingham Heart Study, and the National Cholesterol Education Program Expert Panel on Detection, Evaluation, and Treatment of High Blood Cholesterol in Adults (Adult Treatment Panel III), also known as NCEP/ATP III. Additional biomarkers suitable for biomarker panels include, without limitation and if not already selected, any combination of biomarkers selected from adiponectin, angiotensin II, complement factor 3, leptin, mRNAx, NFκB, IL-6, MMP-9, TNFα, NFκB, eNOS, PPARy, MCP-1, PAI-1, ICAM/VCAM, E-selectin, P-selectin, von Willebrand factor, sCD40L, insulin, proinsulin, glucose, HbAlc, lipids such as free fatty acids, total cholesterol, triglycerides, VLDL, LDL, small dense LDL, oxidized LDL, resistin, HDL, NO, IκB-α, IκB-β, p105, RelA, TNFα, MIF, inflammatory cytokines, molecules involved in signaling pathways, traditional laboratory risk factors and any biomarkers disclosed in US/2008/0057590. Glucose as used herein includes, without limitation, fasting glucose as well as glucose concentrations taken during and after the oral glucose tolerance test, such as 120 minute Glucose. Insulin as used herein includes, without limitation, fasting insulin and insulin concentrations taken during and after the oral glucose tolerance test, such as 120 minute Insulin. Traditional laboratory risk factors are also understood to encompass without limitation, fibrinogen, lipoprotein (a), c-reactive protein (including hsCRP), D-dimer, and homocysteine. It should be understood that in these embodiments, the biomarker panel can include any combination of biomarkers selected from hsCRP, MCP-1, MMP-9 and TNFα and the remainder of these markers.

A biomarker can also be a clinical parameter, although in some embodiments, the biomarker is not included in the definition of “biomarker”. The term “clinical parameter” refers to all non-sample or non-analyte biomarkers of subject health status or other characteristics, such as, without limitation, age, ethnicity, gender, diastolic blood pressure and systolic blood pressure, family history, height, weight, waist and hip circumference, body-mass index, as well as others such as Type I or Type II Diabetes Mellitus or Gestational Diabetes Mellitus (collectively referred to here as Diabetes), resting heart rate, homeostatic model assessment (HOMA), HOMA insulin resistance (HOMA-IR), intravenous glucose tolerance (SI(IVGT)), β-cell function, macrovascular function, microvascular function, atherogenic index, blood pressure, low-density lipoprotein/high-density lipoprotein ratio, intima-media thickness, and UKPDS risk score. Other clinical parameters are disclosed in US/2008/0057590.

In various exemplary embodiments, the biomarker panel comprises hsCRP, MCP-1, MMP-9 and TNFα. In various exemplary embodiments, the biomarker panel comprises any combination of hsCRP, MCP-1, MMP-9 and TNFα. In various exemplary embodiments, the biomarker panel consists of hsCRP, MCP-1, MMP-9 and TNFα. In various exemplary embodiments, the biomarker panel consists of any combination of hsCRP, MCP-1, MMP-9 and TNFα.

In various exemplary embodiments, the biomarker panel comprises or consists of hsCRP, MCP-1, MMP-9 and TNFα and 1, 2, 3, 4 or more additional biomarkers. In various exemplary embodiments, the biomarker panel comprises or consists of any combination of hsCRP, MCP-1, MMP-9 and TNFα and 1, 2, 3, 4 or more additional biomarkers. In various exemplary embodiments, the biomarker panel comprises or consists of hsCRP, MCP-1, MMP-9, TNFα and an NFκB inhibitor (such as IκBα, IκBβ or IκBε).

In various exemplary embodiments, the biomarker panel comprises NFκB (e.g., p105 and relA), NFκB-modulated protein(s) (e.g., MMP9 and IL6) and NFκB inhibitor(s) (e.g., IκBα, IκBβ or IκBε). In various exemplary embodiments, the biomarker panel comprises any combination of NFκB (e.g., p105 and relA), NFκB-modulated protein(s) (e.g., MMP9 and IL6) and NFκB inhibitor(s) (e.g., IκBα, IκBβ or IκBε). In various exemplary embodiments, the biomarker panel consists of NFκB (e.g., p105 and relA), NFκB-modulated protein(s) (e.g., MMP9 and IL6) and NFκB inhibitor(s) (e.g., IκBα, IκBβ or IκBε). In various exemplary embodiments, the biomarker panel consists of any combination of NFκB (e.g., p105 and relA), NFκB-modulated protein(s) (e.g., MMP9 and IL6) and NFκB inhibitor(s) (e.g., IκBα, IκBβ or IκBε).

In various exemplary embodiments, the biomarker panel comprises or consists of NFκB (e.g., p105 and relA), NFκB-modulated protein(s) (e.g., MMP9 and IL6), NFκB inhibitor(s) (e.g., IκBα, IκBβ or IκBε) and 1, 2, 3, 4 or more additional biomarkers. In various exemplary embodiments, the biomarker panel comprises or consists of any combination of NFκB (e.g., p105 and relA), NFκB-modulated protein(s) (e.g., MMP9 and IL6), NFκB inhibitor(s) (e.g., IκBα, IκBβ or IκBε) and 1, 2, 3, 4 or more additional biomarkers.

### Measurement and Detection of Biomarkers

Biomarkers generally can be measured and detected through a variety of assays, methods and detection systems known to one of skill in the art. The term “measuring,” “detecting,” or “taking a measurement” refers to a quantitative or qualitative determination of a property or characteristic of an entity, e.g., quantifying the amount or the activity level of a molecule. The term “concentration” or “level” can refer to an absolute or relative quantity. Measuring a molecule may also include determining the absence or presence of the molecule. A measurement may refer to one observation under a set of conditions or an equally- or differently-weighted average of a plurality of observations under the same set of conditions. Thus, in various embodiments, a measurement of the concentration of a biomarker is derived from one observation of the concentration, and in various embodiments, a measurement of a biomarker is derived from an equally- or differently-weighted average of a plurality of observations of the concentration. In various embodiments, measuring a biomarker panel comprises measuring the concentrations of each member of the biomarker panel in a sample.

Various methods include but are not limited to refractive index spectroscopy (RI), ultra-violet spectroscopy (UV), fluorescence analysis, radiochemical analysis, near-infrared spectroscopy (near-IR), infrared (IR) spectroscopy, nuclear magnetic resonance spectroscopy (NMR), light scattering analysis (LS), mass spectrometry, pyrolysis mass spectrometry, nephelometry, dispersive Raman spectroscopy, gas chromatography, liquid chromatography, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, colorimetry and surface plasmon resonance (such as according to systems provided by Biacore Life Sciences). See also WO/2004/056456 and WO/2004/088309. In this regard, biomarkers can be measured using the above-mentioned detection methods, or other methods known to the skilled artisan. Other biomarkers can be similarly detected using reagents that are specifically designed or tailored to detect them.

Different types of biomarkers and their measurements can be combined in the compositions and methods of the present invention. In various embodiments, the protein form of the biomarkers is measured. In various embodiments, the nucleic acid form of the biomarkers is measured. In exemplary embodiments, the nucleic acid form is mRNA. In various embodiments, measurements of protein biomarkers are used in conjunction with measurements of nucleic acid biomarkers.

Using sequence information provided by the database entries for the biomarker sequences, expression of the biomarker sequences can be detected (if present) and measured using known techniques. For example, sequences in sequence database entries or sequences disclosed herein can be used to construct probes for detecting biomarker RNA sequences in, e.g., Northern blot hybridization analyses or methods which specifically and, preferably, quantitatively amplify specific nucleic acid sequences. As another example, the sequences can be used to construct primers for specifically amplifying the biomarker sequences in, e.g., amplification-based detection methods such as reverse-transcription based polymerase chain reaction (RT-PCR). When alterations in gene expression are associated with gene amplification, deletion, polymorphisms and mutations, sequence comparisons in test and reference populations can be made by comparing relative amounts of the examined DNA sequences in the test and reference cell populations. In addition to Northern blot and RT-PCR, RNA can also be measured using, for example, other target amplification methods (e.g., transcription-mediated amplification (TMA), strand displacement amplification (SDA), nucleic acid sequence based amplification (NASBA) and real time PCR), signal amplification methods (e.g., bDNA), nuclease protection assays, in situ hybridization and the like.

Thus, in one aspect, the invention provides a probe set comprising a capture binding ligand selective for hsCRP, a capture probe selective for MCP-1 nucleic acid, a capture probe selective for MMP-9 nucleic acid and a capture probe selective for TNFα nucleic acid; or a combination thereof.

In one aspect, the invention provides a probe set comprising a capture probe selective for NFκB nucleic acid (e.g., p105 and relA), a capture probe selective for nucleic acid of NFκB-modulated protein(s) (e.g., MMP9 and IL6) and a capture probe selective for nucleic acid of NFκB inhibitor(s) (e.g., IκBα or IκBβ); or a combination thereof.

In one aspect, the invention provides a primer set comprising a primer selective for MCP-1 nucleic acid, a primer selective for MMP-9 nucleic acid and a primer selective for TNFα nucleic acid; or a combination thereof.

In one aspect, the invention provides a primer set comprising a primer selective for NFκB nucleic acid (e.g., p105 and relA), a primer selective for nucleic acid of NFκB-modulated protein(s) (e.g., MMP9 and IL6) and a primer selective for nucleic acid of NFκB inhibitor(s) (e.g., IκBα or IκBβ); or a combination thereof.

A ligand that “specifically binds” or “selectively binds” or is “selective for” a biomarker means that the ligand binds the biomarker with specificity sufficient to differentiate between the biomarker and other components or contaminants of the sample.

Of particular interest for the measurement of biomarkers in the present invention are biochip assays. In one aspect, the invention provides a composition comprising a solid support comprising one or more capture binding ligands, each selective for a different biomarker of a biomarker panel. In various embodiments, the capture ligand is an antibody. In various embodiments, the capture ligand is a nucleic acid. In various embodiments, the composition further comprises a soluble binding ligand for one or more biomarkers of a biomarker panel. In one aspect, the invention provides methods of assaying a sample comprising contacting the sample with a solid support comprising one or more capture binding ligands, each selective for a different biomarker of a biomarker panel.

By “biochip” or “chip” herein is meant a composition generally comprising a solid support or substrate to which a capture binding ligand (also called an adsorbent, affinity reagent or binding ligand, or when nucleic acid is measured, a capture probe) is attached and can bind either proteins, nucleic acids or both.

Generally, where a biochip is used for measurements of protein and nucleic acid biomarkers, the protein biomarkers are measured on a chip separate from that used to measure the nucleic acid biomarkers. For nonlimiting examples of additional platforms and methods useful for measuring nucleic acids, see US/2006/0275782, US/2005/0064469 and DE10201463. In various embodiments, biomarkers are measured on the same platform, such as on one chip. In various embodiments, biomarkers are measured using different platforms and/or different experimental runs.

By “binding ligand,” “capture binding ligand,” “capture binding species,” “capture probe” or “capture ligand” herein is meant a compound that is used to detect the presence of or to quantify, relatively or absolutely, a target analyte, target species or target sequence (all used interchangeably) and that will bind to the target analyte, target species or target sequence. Generally, the capture binding ligand or capture probe allows the attachment of a target species or target sequence to a solid support for the purposes of detection as further described herein. Attachment of the target species to the capture binding ligand may be direct or indirect. In exemplary embodiments, the target species is a biomarker. As will be appreciated by those in the art, the composition of the binding ligand will depend on the composition of the biomarker. Binding ligands for a wide variety of biomarkers are known or can be readily found using known techniques. For example, when the biomarker is a protein, the binding ligands include proteins (particularly including antibodies or fragments thereof (FAbs, etc.) as discussed further below) or small molecules. The binding ligand may also have cross-reactivity with proteins of other species. Antigen-antibody pairs, receptor-ligands, and carbohydrates and their binding partners are also suitable analyte-binding ligand pairs. In various embodiments, the binding ligand may be nucleic acid. Nucleic acid binding ligands find particular use when proteins are the targets; alternatively, as is generally described in U.S. Pat. Nos. 5,270,163; 5,475,096; 5,567,588; 5,595,877; 5,637,459; 5,683,867; 5,705,337 and related patents, hereby incorporated by reference, nucleic acid “aptamers” can be developed for binding to virtually any biomarker. Nucleic acid binding ligands also find particular use when nucleic acids are binding targets. There is a wide body of literature relating to the development of binding partners based on combinatorial chemistry methods. In these embodiments, when the binding ligand is a nucleic acid, preferred compositions and techniques are outlined in WO/1998/020162, hereby incorporated by reference.

Capture binding ligands that are useful in the present invention may be “selective” for, “specifically bind” or “selectively bind” their target, such as a protein. Typically, specific or selective binding can be distinguished from non-specific or non-selective binding when the dissociation constant (K_{D}) is less than about 1×10⁻⁵M or less than about 1×10⁻⁶ M or 1×10⁻⁷M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

In various exemplary embodiments, the capture binding ligand is an antibody. These embodiments are particularly useful for the detection of the protein form of a biomarker.

Detecting or measuring the concentration (e.g. to determine transcription level) of a biomarker involves binding of the biomarker to a capture binding ligand, generally referred to herein as a “capture probe” when the nucleic acid form (e.g. mRNA) of the biomarker is to be detected on a solid support. In that sense, the biomarker is a target sequence. The term “target sequence” or “target nucleic acid” or grammatical equivalents herein means a nucleic acid sequence that may be a portion of a gene, a regulatory sequence, genomic DNA, cDNA, RNA including mRNA and rRNA, or others. As is outlined herein, the target sequence may be a target sequence from a sample. The target sequence may in some embodiments be a secondary target such as a product of an amplification reaction such as PCR etc. In some embodiments, measuring a nucleic acid can thus refer to measuring the complement of the nucleic acid. It may be any length, with the understanding that longer sequences are more specific.

Capture probes that “selectively bind” (i.e., are “complementary” or “substantially complementary”) to or are “selective for” a target nucleic acid find use in the present invention. “Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single stranded RNA or DNA molecules may be said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa, Nucleic Acids Res., 2004, 12: 203.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. In one embodiment, stable duplex means that a duplex structure is not destroyed by a stringent wash, e.g. conditions including temperature of about 5° C. less that the Tₘ of a strand of the duplex and low monovalent salt concentration, e.g. less than 0.2 M, or less than 0.1 M. “Perfectly matched” in reference to a duplex means that the poly- or oligonucleotide strands making up the duplex form a double stranded structure with one another such that every nucleotide in each strand undergoes Watson-Crick basepairing with a nucleotide in the other strand. The term “duplex” includes the pairing of nucleoside analogs, such as deoxyinosine, nucleosides with 2-aminopurine bases, PNAs, and the like, that may be employed. A “mismatch” in a duplex between two oligonucleotides or polynucleotides means that a pair of nucleotides in the duplex fails to undergo Watson-Crick bonding.

The target sequence may also comprise different target domains; for example, a first target domain of the sample target sequence may hybridize to a first capture probe, a second target domain may hybridize to a label probe (e.g. a “sandwich assay” format), etc. The target domains may be adjacent or separated as indicated. Unless specified, the terms “first” and “second” are not meant to confer an orientation of the sequences with respect to the 5′-3′ orientation of the target sequence. For example, assuming a 5′-3′ orientation of the target sequence, the first target domain may be located either 5′ to the second domain, or 3′ to the second domain.

When nucleic acids are used as the target analyte, the assays of the invention can take on a number of embodiments. In one embodiment, the assays are done in a solution format. In one embodiment, end-point or real time PCR formats are used, as are well known in the art. These assays can be done either as a panel, in individual tubes or wells, or as multiplex assays, using sets of primers and different labels within a single tube or well. qPCR techniques relying on 5′ nuclease assays using FRET probes or intercalating dyes such as SYBR Green can also be used for nucleic acid targets. In addition to PCR-based solution formats, other formats can be utilized, including, but not limited to for example ligation based assays utilizing FRET dye pairs. In this embodiment, only upon ligation of two (or more) probes hybridized to the target sequence is a signal generated.

In many embodiments, the assays are done on a solid support, utilizing a capture probe associated with the surface. As discussed herein, the capture probes (or capture binding ligands, as they are sometimes referred to) can be covalently attached to the surface, for example using capture probes terminally modified with functional groups, for example amino groups, that are attached to modified surfaces such as silanized glass. Alternatively, non-covalent attachment, such as electrostatic, hydrophobic/hydrophilic adhesion can be utilized. As is appreciated by those in the art and discussed herein, a large number of attachments are possible on a wide variety of surfaces.

In one embodiment, the target sequence comprises a detectable label, as described herein. In this embodiment, the label is generally added to the target sequence during amplification of the target in one of two ways: either labeled primers are utilized during the amplification step or labeled dNTPs are used, both of which are well known in the art.

The detectable label can either be a primary or secondary label as discussed herein. For example, in one embodiment, the label on the primer and/or a dNTP is a primary label such as a fluorophore. In other words, a primary label produces a detectable signal that can be directly detected. By “label” or “labeled” herein is meant that a compound has at least one molecule, element, isotope or chemical compound attached to enable the detection of the compound. In general, labels fall into four classes: a) isotopic labels, which may be radioactive or heavy isotopes; b) magnetic, electrical, thermal; c) colored or luminescent dyes; and d) enzymes; although labels include particles such as magnetic particles as well. The dyes may be chromophores or phosphors but are preferably fluorescent dyes, which due to their strong signals provide a good signal-to-noise ratio for decoding. Suitable dyes for use in the invention include, but are not limited to, fluorescent lanthanide complexes, including those of europium and terbium, fluorescein, rhodamine, tetramethylrhodamine, eosin, erythrosin, coumarin, methyl-coumarins, pyrene, Malacite green, stilbene, Lucifer Yellow, Cascade Blue, Texas Red, Alexa dyes and others described in Molecular Probes Handbook (6th ed.) by Richard P. Haugland. Additional labels include nanocrystals or Q-dots as described in U.S. Pat. No. 6,544,732.

Alternatively, the label may be a secondary label, such as biotin or an enzyme. A secondary label requires additional reagents that lead to the production of a detectable signal. A secondary label is one that is indirectly detected; for example, a secondary label can bind or react with a primary label for detection, can act on an additional product to generate a primary label (e.g. enzymes), or may allow the separation of the compound comprising the secondary label from unlabeled materials, etc. Secondary labels include, but are not limited to, one of a binding partner pair; chemically modifiable moieties; nuclease inhibitors, enzymes such as horseradish peroxidase, alkaline phosphatases, luciferases, etc. Secondary labels can also include additional labels.

In one embodiment, the primers or dNTPs are labeled with biotin, and then a streptavidin/label complex is added. In one embodiment, the streptavidin/label complex contains a label such as a fluorophore. In an alternative embodiment, the streptavidin/label complex comprises an enzymatic label. For example, the label complex can comprise horseradish peroxidase, and upon addition of a precipitating agent, such as TMB, the action of the horseradish peroxidase causes an optically detectable precipitation reaction. This has a particular benefit in that the optics for detection does not require the use of a fluorimeter or other detector, which can add to the expense of carrying out the methods.

In various embodiments, the secondary label is a binding partner pair. For example, the label may be a hapten or antigen, which will bind its binding partner. Suitable binding partner pairs include, but are not limited to: antigens (such as a polypeptide) and antibodies (including fragments thereof (FAbs, etc.)); other polypeptides and small molecules, including biotin/streptavidin; enzymes and substrates or inhibitors; other protein-protein interacting pairs; receptor-ligands; and carbohydrates and their binding partners. Nucleic acid—nucleic acid binding proteins pairs are also useful. In general, the smaller of the pair is attached to the NTP for incorporation into the primer. Preferred binding partner pairs include, but are not limited to, biotin (or imino-biotin) and streptavidin, digeoxinin and Abs, and Prolinx™ reagents.

In the sandwich formats of the invention, an enzyme serves as the secondary label, bound to the soluble capture ligand. Of particular use in some embodiments is the use of horseradish peroxidase, which when combined with a precipitating agent such as 3,3′,5,5′-tetramethylbenzidine (TMB) forms a colored precipitate which is then detected. In some cases, the soluble capture ligand comprises biotin, which is then bound to a enzyme-streptavidin complex and forms a colored precipitate with the addition of TMB.

Thus, in various embodiments, the detectable label or detectable marker is a conjugated enzyme (for example, horseradish peroxidase). In various embodiments, the system relies on detecting the precipitation of a reaction product or on a change in, for example, electronic properties for detection. In various embodiments, none of the compounds comprises a label.

In alternate embodiments, the solid phase assay relies on the use of a labeled soluble capture ligand, sometimes referred to as a “label probe” or “signaling probe” when the target analyte is a nucleic acid. In this format, the assay is a “sandwich” type assay, where the capture probe binds to a first domain of the target sequence and the label probe binds to a second domain. In this embodiment, the label probe can also be either a primary (e.g. a fluorophore) or a secondary (biotin or enzyme) label. In one embodiment, the label probe comprises biotin, and a streptavidin/enzyme complex is used, as discussed herein. As above, for example, the complex can comprise horseradish peroxidase, and upon addition of TMB, the action of the horseradish peroxidase causes an optically detectable precipitation reaction t.

In embodiments finding particular use herein, a sandwich format is utilized, in which target species are unlabeled. In these embodiments, a “capture” or “anchor” binding ligand is attached to the detection surface as described herein, and a soluble binding ligand (frequently referred to herein as a “signaling probe,” “label probe” or “soluble capture ligand”) binds independently to the target species and either directly or indirectly comprises at least one label or detectable marker.

As used herein, the term “fluorescent signal generating moiety” or “fluorophore” refers to a molecule or part of a molecule that absorbs energy at one wavelength and re-emits energy at another wavelength. Fluorescent properties that can be measured include fluorescence intensity, fluorescence lifetime, emission spectrum characteristics, energy transfer, and the like.

Signals from single molecules can be generated and detected by a number of detection systems, including, but not limited to, scanning electron microscopy, near field scanning optical microscopy (NSOM), total internal reflection fluorescence microscopy (TIRFM), and the like. Abundant guidance is found in the literature for applying such techniques for analyzing and detecting nanoscale structures on surfaces, as evidenced by the following references that are incorporated by reference: Reimer et al, editors, Scanning Electron Microscopy: Physics of Image Formation and Microanalysis, 2nd Edition (Springer, 1998); Nie et al, Anal. Chem., 78: 1528-1534 (2006); Hecht et al, Journal Chemical Physics, 112: 7761-7774 (2000); Zhu et al, editors, Near-Field Optics: Principles and Applications (World Scientific Publishing, Singapore, 1999); Drmanac, WO/2004/076683; Lehr et al, Anal. Chem., 75: 2414-2420 (2003); Neuschafer et al, Biosensors & Bioelectronics, 18: 489-497 (2003); Neuschafer et al, U.S. Pat. No. 6,289,144; and the like.

Thus, a detection system for fluorophores includes any device that can be used to measure fluorescent properties as discussed above. In various embodiments, the detection system comprises an excitation source, a fluorophore, a wavelength filter to isolate emission photons from excitation photons and a detector that registers emission photons and produces a recordable output, in some embodiments as an electrical signal or a photographic image. Examples of detection devices include without limitation spectrofluorometers and microplate readers, fluorescence microscopes, fluorescence scanners (including e.g. microarray readers) and flow cytometers.

The term “solid support” or “substrate” refers to any material that can be modified to contain discrete individual sites appropriate for the attachment or association of a capture binding ligand. Suitable substrates include metal surfaces such as gold, electrodes, glass and modified or functionalized glass, plastics (including acrylics, polystyrene and copolymers of styrene and other materials, polypropylene, polyethylene, polybutylene, polycarbonate, polyurethanes, Teflon, derivatives thereof, etc.), polysaccharides, nylon or nitrocellulose, resins, mica, silica or silica-based materials including silicon and modified silicon, carbon, metals, inorganic glasses, fiberglass, ceramics, GETEK (a blend of polypropylene oxide and fiberglass) and a variety of other polymers. Of particular use in the present invention are the ClonDiag materials described below.

In one aspect, the invention provides a solid support comprising or consisting of capture binding ligands selective for the protein form of the members of a biomarker panel. In one aspect, the invention provides a solid support comprising or consisting of capture probes selective for the nucleic acid form of the members of a biomarker panel.

Frequently, the surface of a biochip comprises a plurality of addressable locations, each of which comprises a capture binding ligand. An “array location,” “addressable location,” “pad” or “site” herein means a location on the substrate that comprises a covalently attached capture binding ligand. An “array” herein means a plurality of capture binding ligands in a regular, ordered format, such as a matrix. The size of the array will depend on the composition and end use of the array. Arrays containing from about two or more different capture binding ligands to many thousands can be made. Generally, the array will comprise a plurality of types of capture binding ligands depending on the end use of the array. In the present invention, the array can include controls, replicates of the markers and the like. Exemplary ranges are from about 3 to about 50. In some embodiments, the compositions of the invention may not be in array format; that is, for some embodiments, compositions comprising a single capture ligand may be made as well. In addition, in some arrays, multiple substrates may be used, either of different or identical compositions. Thus for example, large arrays may comprise a plurality of smaller substrates.

A number of different biochip array platforms as known in the art may be used. For example, the compositions and methods of the present invention can be implemented with array platforms such as GeneChip (Affymetrix), CodeLink Bioarray (Amersham), Expression Array System (Applied Biosystems), SurePrint microarrays (Agilent), Sentrix LD BeadChip or Sentrix Array Matrix (Illumina) and Verigene (Nanosphere).

In various exemplary embodiments, detection and measurement of biomarkers utilizes colorimetric methods and systems in order to provide an indication of binding of a target analyte or target species. In colorimetric methods, the presence of a bound target species such as a biomarker will result in a change in the absorbance or transmission of light by a sample or substrate at one or more wavelengths. Detection of the absorbance or transmission of light at such wavelengths thus provides an indication of the presence of the target species.

A detection system for colorimetric methods includes any device that can be used to measure colorimetric properties as discussed above. Generally, the device is a spectrophotometer, a colorimeter or any device that measures absorbance or transmission of light at one or more wavelengths. In various embodiments, the detection system comprises a light source; a wavelength filter or monochromator; a sample container such as a cuvette or a reaction vial; a detector, such as a photoresistor, that registers transmitted light; and a display or imaging element. In some embodiments, a change in the colorimetric properties of a sample can be detected directly by the naked eye, i.e., by direct visual inspection.

In various exemplary embodiments, a ClonDiag chip platform is used for the colorimetric detection of biomarkers. In various embodiments, a ClonDiag ArrayTube (AT) is used. One unique feature of the ArrayTube is the combination of a micro probe array (the biochip) and micro reaction vial. In various embodiments, where a target sequence is a nucleic acid, detection of the target sequence is done by amplifying and biotinylating the target sequence contained in a sample and optionally digesting the amplification products. The amplification product is then allowed to hybridize with probes contained on the ClonDiag chip. A solution of a streptavidin-enzyme conjugate, such as Poly horseradish peroxidase (HRP) conjugate solution, is contacted with the ClonDiag chip. After washing, a dye solution such as o-dianisidine substrate solution is contacted with the chip. Oxidation of the dye results in precipitation that can be detected colorimetrically. Further description of the ClonDiag platform is found in Monecke S, Slickers P, Hotzel H et al., Clin Microbiol Infect 2006, 12: 718-728; Monecke S, Berger-Bachi B, Coombs C et al., Clin Microbiol Infect 2007, 13: 236-249; Monecke S, Leube I and Ehricht R, Genome Lett 2003, 2: 106-118; German Patent DE 10201463; US Publication US/2005/0064469 and ClonDiag, ArrayTube (AT) Experiment Guideline for DNA-Based Applications, version 1.2, 2007, all incorporated by reference in their entirety. Use of the ClonDiag platform for genotyping is described in Sachse K et al., BMC Microbiology 2008, 8: 63; Monecke S and Ehricht R, Clin Microbiol Infect 2005, 11: 825-833; and Monecke S et al., Clin Microbiol Infect 2008, 14(6): 534-545. One of skill in the art will appreciate that numerous other dyes that react with a peroxidase can be utilized to produce a colorimetric change, such as 3,3′,5,5′-tetramethylbenzidine (TMB). For information on specific assay protocols, see www.clondiag.com/technologies/publications.php. Such dyes may be referred to as a precipitating agent herein.

In various embodiments, where a target species is a protein, the ArrayTube biochip comprises capture binding ligands such as antibodies. A sample is contacted with the biochip, and any target species present in the sample is allowed to bind to the capture binding ligand antibodies. A soluble capture binding ligand or a detection compound such as a horseradish peroxidase conjugated antibody is allowed to bind to the target species. A dye, such as TMB, is then added and allowed to react with the horseradish peroxidase, causing precipitation and a color change that is detected by a suitable detection device. Further description of protein detection using ArrayTube is found in, for example, Huelseweh B, Ehricht R and Marschall H-J, Proteomics, 2006, 6, 2972-2981; and ClonDiag, ArrayTube (AT) Experiment Guideline for Protein-Based Applications, version 1.2, 2007, all incorporated by reference in their entirety.

Transmission detection and analysis is performed with a ClonDiag AT reader instrument. Suitable reader instruments and detection devices include the ArrayTube Workstation ATS and the ATR 03.

In addition to ArrayTube, the ClonDiag ArrayStrip (AS) can be used. The ArrayStrip provides a 96-well format for high volume testing. Each ArrayStrip consists of a standard 8-well strip with a microarray integrated into the bottom of each well. Up to 12 ArrayStrips can be inserted into one microplate frame enabling the parallel multiparameter testing of up to 96 samples. The ArrayStrip can be processed using the ArrayStrip Processor ASP, which performs all liquid handling, incubation, and detection steps required in array based analysis. In various embodiments, where a protein is detected, a method of using the ArrayStrip to detect the protein comprises conditioning the AS array with buffer or blocking solution; loading of up to 96 sample solutions in the AS wells to allow for binding of the protein; 3× washing; conjugating with a secondary antibody linked to HRP; 3× washing; precipitation staining with TMB; and AS array imaging and optional data storage.

Those skilled in the art will be familiar with numerous additional immunoassay formats and variations thereof which may be useful for carrying out the method disclosed herein. See generally E. Maggio, Enzyme-Immunoassay, (CRC Press, Inc., Boca Raton, Fla., 1980); see also U.S. Pat. Nos. 4,727,022; 4,659,678; 4,376,110; 4,275,149; 4,233,402; and 4,230,767.

In general, immunoassays carried out in accordance with the present invention may be homogeneous assays or heterogeneous assays. In a homogeneous assay the immunological reaction usually involves the specific antibody (e.g., anti-biomarker protein antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution Immunochemical labels which may be employed include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays include immunoblotting, immunofluorescence methods, immunoprecipitation, chemiluminescence methods, electrochemiluminescence (ECL) or enzyme-linked immunoassays.

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein, rhodamine) in accordance with known techniques.

As used herein, the term “antibody” means a protein comprising one or more polypeptides substantially encoded by all or part of the recognized immunoglobulin genes. The recognized immunoglobulin genes, for example in humans, include the kappa (κ), lambda (λ) and heavy chain genetic loci, which together compose the myriad variable region genes, and the constant region genes mu (μ), delta (δ), gamma (γ), epsilon (ε) and alpha (α), which encode the IgM, IgD, IgG, IgE, and IgA isotypes respectively. Antibody herein is meant to include full length antibodies and antibody fragments, and may refer to a natural antibody from any organism, an engineered antibody or an antibody generated recombinantly for experimental, therapeutic or other purposes as further defined below. Antibody fragments include Fab, Fab′, F(ab′)₂, Fv, scFv or other antigen-binding subsequences of antibodies and can include those produced by the modification of whole antibodies or those synthesized de novo using recombinant DNA technologies. The term “antibody” refers to both monoclonal and polyclonal antibodies. Antibodies can be antagonists, agonists, neutralizing, inhibitory or stimulatory.

The invention further provides kits for performing any of the methods disclosed herein for a number of medical (including diagnostic and therapeutic), industrial, forensic and research applications. Kits may comprise a carrier, such as a box, carton, tube or the like, having in close confinement therein one or more containers, such as vials, tubes, ampoules, bottles, pouches, envelopes and the like. In various embodiments, a kit comprises one or more components selected from one or more media or media ingredients and reagents for the measurement of the various biomarkers and biomarker panels disclosed herein. For example, kits of the invention may also comprise, in the same or different containers, in any combination, one or more DNA polymerases, one or more primers, one or more probes, one or more binding ligands, one or more suitable buffers, one or more nucleotides (such as deoxynucleoside triphosphates (dNTPs) and preferably labeled dNTPs), one or more detectable labels and markers and one or more solid supports, any of which is described herein. The components may be contained within the same container, or may be in separate containers to be admixed prior to use. The kits of the present invention may also comprise one or more instructions or protocols for carrying out the methods of the present invention. The kits may comprise a detector for detecting a signal generated through use of the components of the invention in conjunction with a sample. The kits may also comprise a computer or a component of a computer, such as a computer-readable storage medium or device. Examples of storage media include, without limitation, optical disks such as CD, DVD and Blu-ray Discs (BD); magneto-optical disks; magnetic media such as magnetic tape and internal hard disks and removable disks; semi-conductor memory devices such as EPROM, EEPROM and flash memory; and RAM. The computer-readable storage medium may comprise software encoding references to the various therapies and treatment regimens disclosed herein. The software may be interpreted by a computer to provide the practitioner with treatments according to various measured concentrations of biomarkers as provided herein. In various embodiments, the kit comprises a biomarker assay involving a lateral-flow-based point-of-care rapid test with detection of risk thresholds, or a biochip with quantitative assays for the constituent biomarkers. Generally, any of the methods disclosed herein can comprise using any of the kits (comprising primers, probes, labels, ligands and solid supports in any combination) disclosed herein.

In one aspect, the invention provides a kit comprising a solid support comprising or consisting of capture binding ligands selective for the protein form of the members of a biomarker panel. In one aspect, the invention provides a kit comprising a solid support comprising or consisting of capture probes selective for the nucleic acid form of the members of a biomarker panel. In one aspect, the invention provides a kit comprising (a) a solid support comprising or consisting of capture binding ligands selective for the protein form of the members of a biomarker panel and (b) a solid support comprising or consisting of capture probes selective for the nucleic acid form of the members of a biomarker panel.

In one aspect, the invention provides use of a kit comprising a solid support comprising probes selective for members of a biomarker panel for determining a second therapy for a subject that has undergone a first therapy, wherein the subject is suffering from a disease (e.g. atherosclerosis). In one embodiment, the use comprises (a) contacting a first sample from the subject with a solid support of the kit; (b) taking a first measurement of the concentrations of the biomarker panel in the sample; (c) effecting a first therapy on the subject; (d) contacting a second sample from the subject with the solid support of the kit; and (e) making a comparison of the first and second measurements.

In one aspect, the invention provides use of a kit comprising a solid support comprising probes selective for members of a biomarker panel for determining whether a subject belongs to a population that would benefit from a second therapy, wherein the subject has undergone a first therapy. In one embodiment, the use comprises (a) contacting a first sample from the subject with a solid support of the kit; (b) taking a first measurement of the concentrations of the biomarker panel in the sample; (c) effecting a first therapy on the subject; (d) contacting a second sample from the subject with the solid support of the kit; and (e) making a comparison of the first and second measurements.

Using any of the methods and compositions described herein, a sample can be assayed to determine concentrations of a biomarker panel. Thus, in one aspect, the invention provides a method of assaying a sample comprising taking a measurement of a biomarker panel in the sample. In one aspect, the invention provides a method of acquiring data relating to a sample comprising taking a measurement of a biomarker panel in the sample. In one aspect, the invention provides a method of measuring analyte concentrations in a sample comprising taking a measurement of a biomarker panel in the sample. In one aspect, the invention provides a method of determining mononuclear cell activation in a sample comprising taking a measurement of a biomarker panel in the sample. In some embodiments, the method comprises contacting the sample with a composition comprising a solid support comprising a capture binding ligand or capture probe for each biomarker of a biomarker panel. Any biomarker panel disclosed herein can be used in these and other methods.

### Methods of Diagnosing and Treating

The compositions and methods of the present invention can be used in the prognosis, diagnosis and treatment of disease in a subject.

A “subject” in the context of the present invention is an animal, preferably a mammal. The mammal can be a human, non-human primate, mouse, rat, dog, cat, horse, or cow, but are not limited to these examples. In various exemplary embodiments, a subject is human and may be referred to as a “patient”. Mammals other than humans can be advantageously used as subjects that represent animal models of a disease or for veterinarian applications. A subject can be one who has been previously diagnosed or identified as having a disease, and optionally has already undergone, or is undergoing, a therapeutic intervention for a disease. Alternatively, a subject can also be one who has not been previously diagnosed as having a disease. For example, a subject can be one who exhibits one or more risk factors for a disease, or one who does not exhibit a disease risk factor, or one who is asymptomatic for a disease. A subject can also be one who is suffering from or at risk of developing a disease. In certain embodiments, the subject can be already undergoing therapy or can be a candidate for therapy. In some embodiments, the patient is being evaluated to see whether treatment with an insulin sensitizer drug is efficacious in the patient.

The invention provides compositions and methods for laboratory and point-of-care tests for measuring biomarkers in a sample from a subject. The invention can be generally applied for a number of different diseases. In exemplary embodiments, the disease is insulin resistance. In exemplary embodiments, the disease is cardiovascular disease or risk. In exemplary embodiments, the disease is atherosclerosis. In exemplary embodiments, the disease is diabetes mellitus. In exemplary embodiments, the disease is cardiodiabetes.

The panel of biomarkers disclosed herein may find particular use for in diagnosing and treating disorders associated with cardiodiabetes. “Cardiodiabetes” refers to patients with insulin resistance and β-cell dysfunction without elevation of blood glucose who are not identified as suffering from diabetes mellitus. These normoglycemic patients, however, experience the same elevated cardiovascular risk, which is predominantly linked to vascular insulin resistance. A cardiodiabetic subject might not exhibit one or more of the normal symptoms of type 2 diabetes including, but not limited to, hyperglycemia, fatigue, weight gain, excessive eating, poor wound healing and infections. A cardiodiabetic subject is at high risk for cardiovascular disease and may experience events such as myocardial infarction and stroke. That is, diabetes mellitus, cardiodiabetes and metabolic syndrome are phenotypes of a common underlying pathophysiology.

The biomarkers and biomarker panels disclosed herein can be used in methods to diagnose, identify or screen subjects that have, do not have or are at risk for having disease; to monitor subjects that are undergoing therapies for disease; to determine or suggest a new therapy or a change in therapy; to differentially diagnose disease states associated with the disease from other diseases or within sub-classifications of disease; to evaluate the severity or changes in severity of disease in a subject; to stage a subject with the disease and to select or modify therapies or interventions for use in treating a subject with the disease. In an exemplary embodiment, the methods of the present invention are used to identify and/or diagnose subjects who are asymptomatic or presymptomatic for a disease. In this context, “asymptomatic” or “presymptomatic” means not exhibiting the traditional symptoms or enough abnormality for disease. In exemplary embodiments, the subject is normoglycemic.

In one aspect, the invention provides a method of determining a prognosis of a disease in a subject, diagnosing a disease in a subject, or treating a disease in a subject comprises taking a measurement of a biomarker panel in a sample from the subject.

The term “disease status” includes any distinguishable manifestation of the disease, including non-disease. For example, disease status includes, without limitation, the presence or absence of disease, the risk of developing disease, the stage of the disease, the progression of disease (e.g., progress of disease or remission of disease over time), the severity of disease and the effectiveness or response to treatment of disease.

As will be appreciated by those in the art, the biomarkers may be measured in using several techniques designed to achieve more predictable subject and analytical variability. On subject variability, many of the above biomarkers are commonly measured in a fasting state, commonly in the morning, providing a reduced level of subject variability due to both food consumption and metabolism and diurnal variation. All fasting and temporal-based sampling procedures using the biomarkers described herein may be useful for performing the invention. Pre-processing adjustments of biomarker results may also be intended to reduce this effect.

The term “sample” used herein refers to a specimen or culture obtained from a subject and includes fluids, gases and solids including for example tissue. In various exemplary embodiments, the sample comprises blood. Fluids obtained from a subject include for example whole blood or a blood derivative (e.g. serum, plasma, or blood cells), ovarian cyst fluid, ascites, lymphatic, cerebrospinal or interstitial fluid, saliva, mucous, sputum, sweat, urine, or any other secretion, excretion, or other bodily fluids. As will be appreciated by those in the art, virtually any experimental manipulation or sample preparation steps may have been done on the sample. For example, wash steps may be applied to a sample. In various embodiments, a biomarker panel is measured directly in a subject without the need to obtain a separate sample from the patient.

In one aspect, the invention provides a method of diagnosing a subject for a disease comprising taking a measurement of a biomarker panel in a sample from the subject; and correlating the measurement with the disease. The term “correlating” generally refers to determining a relationship between one type of data with another or with a state. In various embodiments, correlating the measurement with disease comprises comparing the measurement with a reference biomarker profile or some other reference value. In various embodiments, correlating the measurement with disease comprises determining whether the subject is currently in a state of disease.

The quantity or activity measurements of a biomarker panel can be compared to a reference value. Differences in the measurements of biomarkers in the subject sample compared to the reference value are then identified. In exemplary embodiments, the reference value is given by a risk category as described further below.

In various embodiments, the reference value is a baseline value. A baseline value is a composite sample of an effective amount of biomarkers from one or more subjects who do not have a disease, who are asymptomatic for a disease or who have a certain level of a disease. A baseline value can also comprise the amounts of biomarkers in a sample derived from a subject who has shown an improvement in risk factors of a disease as a result of treatments or therapies. In these embodiments, to make comparisons to the subject-derived sample, the amounts of biomarkers are similarly calculated. A reference value can also comprise the amounts of biomarkers derived from subjects who have a disease confirmed by an invasive or non-invasive technique, or are at high risk for developing a disease. Optionally, subjects identified as having a disease, or being at increased risk of developing a disease are chosen to receive a therapeutic regimen to slow the progression of a disease, or decrease or prevent the risk of developing a disease. A disease is considered to be progressive (or, alternatively, the treatment does not prevent progression) if the amount of biomarker changes over time relative to the reference value, whereas a disease is not progressive if the amount of biomarkers remains constant over time (relative to the reference population, or “constant” as used herein). The term “constant” as used in the context of the present invention is construed to include changes over time with respect to the reference value.

The biomarkers of the present invention can be used to generate a “reference biomarker profile” of those subjects who do not have a disease according to a certain threshold, are not at risk of having a disease or would not be expected to develop a disease. The biomarkers disclosed herein can also be used to generate a “subject biomarker profile” taken from subjects who have a disease or are at risk for having a disease. The subject biomarker profiles can be compared to a reference biomarker profile to diagnose or identify subjects at risk for developing a disease, to monitor the progression of disease, as well as the rate of progression of disease, and to monitor the effectiveness of disease treatment modalities. The reference and subject biomarker profiles of the present invention can be contained in a machine-readable medium, such as but not limited to, analog tapes like those readable by a VCR; optical media such as CD-ROM, DVD-ROM and the like; and solid state memory, among others.

The biomarker panels of the invention can be used by a practitioner to determine and effect appropriate therapies with respect to a subject given the disease status indicated by measurements of the biomarkers in a sample from the subject. Thus, in one aspect, the invention provides a method of treating a disease in a subject comprising taking a measurement of a biomarker panel in a sample from the subject, and effecting a therapy with respect to the subject. In one embodiment, the concentrations of the biomarkers of the biomarker panel increase or decrease according to the values described herein or stay the same in response to the therapy.

The terms “therapy” and “treatment” may be used interchangeably. In certain embodiments, the therapy can be selected from, without limitation, initiating therapy, continuing therapy, modifying therapy or ending therapy. A therapy also includes any prophylactic measures that may be taken to prevent disease.

In certain embodiments, a therapy comprises administering a disease-modulating drug to a subject. Various examples of suitable disease-modulating drugs are described below. The drug can be a therapeutic or prophylactic used in subjects diagnosed or identified with a disease or at risk of having the disease. In certain embodiments, modifying therapy refers to altering the duration, frequency or intensity of therapy, for example, altering dosage levels.

In various embodiments, effecting a therapy comprises causing a subject to or communicating to a subject the need to make a change in lifestyle, for example, increasing exercise, changing diet, reducing or eliminating smoking and so on. The therapy can also include surgery, for example, bariatric surgery.

Measurement of biomarker concentrations allows for the course of treatment of a disease to be monitored. The effectiveness of a treatment regimen for a disease can be monitored by detecting one or more biomarkers in an effective amount from samples obtained from a subject over time and comparing the amount of biomarkers detected. For example, a first sample can be obtained prior to the subject receiving treatment and one or more subsequent samples are taken after or during treatment of the subject. Changes in biomarker concentrations across the samples may provide an indication as to the effectiveness of the therapy.

To identify therapeutics or drugs that are appropriate for a specific subject, a test sample from the subject can be exposed to a therapeutic agent or a drug, and the concentration of one or more biomarkers can be determined Biomarker concentrations can be compared to a sample derived from the subject before and after treatment or exposure to a therapeutic agent or a drug, or can be compared to samples derived from one or more subjects who have shown improvements relative to a disease as a result of such treatment or exposure.

### Drug Treatments

In exemplary embodiments, effecting a therapy with respect to a subject comprises administering a disease-modulating drug to the subject. The drug may be in any form suitable for administration to a subject, such forms including salts, prodrugs and solvates. The drug may be formulated in any manner suitable for administration to a subject, often according to various known formulations in the art or as disclosed or referenced herein. For example, the drug may be a component of a pharmaceutical composition comprising the drug and an excipient. Any drug, combination of drugs or formulation thereof disclosed herein may be administered to a subject to treat a disease.

The subject may be treated with one or more disease-modulating drugs until altered concentrations of the measured biomarkers return to a baseline value measured in a population not suffering from the disease, experiencing a less severe stage or form of a disease or showing improvements in disease biomarkers as a result of treatment with a disease-modulating drug. Additionally, improvements related to a changed concentration of a biomarker or clinical parameter may be the result of treatment with a disease-modulating drug and may include, for example, a reduction in body mass index (BMI), a reduction in total cholesterol concentrations, a reduction in LDL concentrations, an increase in HDL concentrations, a reduction in systolic and/or diastolic blood pressure, or combinations thereof.

A number of compounds such as a disease-modulating drug may be used to treat a subject and to monitor progress using the methods of the invention. In certain embodiments, the disease-modulating drug comprises an antiobesity drug, a β-blocker, an angiotensin-converting enzyme (ACE) inhibitor, a diuretic, a calcium channel blocker, an angiotensin II receptor blocker, a antiplatelet agent, an anti-DB coagulant agent, a sulfonylurea (SU), a biguanide, an insulin, a glitazone (thiazolidinedione (TZD)), a nitrate, a non-steroidal anti-inflammatory agent, a statin, cilostazol, pentoxifylline, buflomedil or naftidrofuryl. In addition, any combination of these drugs may be administered.

The beneficial effects of these and other drugs can be visualized by assessment of clinical and laboratory biomarkers. For example, results from PROactive (Pfiitzner et al., Expert Review of Cardiovascular Therapy, 2006, 4: 445-459) and recent metanalyses have shown that these surrogate changes may translate into effective reduction of macrovascular risk in patients with type 2 diabetes mellitus.

Insulin sensitizer drugs are particularly useful in the various compositions and methods of the invention. An “insulin sensitizer” as used herein refers to any drug that enhances a subject's response to insulin. Exemplary insulin sensitizers act as agonists to PPAR, in particular to PPARγ. General classes of insulin sensitizers include, without limitation, glitazones (also referred to as thiazolidinediones(TZD)) and glitazars. In some embodiments, metformin is considered to be an insulin sensitizer.

Accordingly, in exemplary embodiments, an insulin sensitizer is administered to a subject to treat a disease. Numerous insulin sensitizers are known in the art and are useful in the present invention. Specific examples of insulin sensitizers include pioglitazone, rosiglitazone, netoglitazone (MCC-555), balaglitazone (DRF-2593), rivoglitazone (CS-011), troglitazone, MB-13.1258, 5-(2,4-dioxothiazolidin-5-ylmethyl)-2-methoxy-N-[4-(trifluoromethyl)benzyl]benzamide (KRP-297), FK-614, compounds described in WO/1999/058510 (e.g. (E)-4-[4-(5-methyl-2-phenyl-4-oxazolylmethoxy) benzyloxyimino]-4-phenylbutyric acid), aleglitazar, farglitazar 262570), tesaglitazar (AZ-242), ragaglitazar (NN-622), muraglitazar (BMS-298585), reglitazar (JTT-501), ONO-5816, LM-4156, metaglidasen (MBX-102), naveglitazar (LY-519818), MX-6054, LY-510929, T-131, THR-0921 and the like. See WO/2005/041962 and US/2006/0280794.

In various exemplary embodiments, a glitazone is administered to a subject to treat a disease. In various exemplary embodiments, pioglitazone is administered to a subject. These and other drugs that are administered to treat a subject have been shown to affect concentrations of various biomarkers.

Furthermore, a glitazone such as pioglitazone may also be administered with other drugs. In various embodiments, pioglitazone is administered with a statin, including but not limited to simvastatin. In various embodiments, pioglitazone may be administered with insulin or a GLP-1 analog, such as exenatide. In various embodiments, pioglitazone may be administered with an oral antidiabetic drug, including but not limited to a sulfonylurea (such as glimepiride), a biguanide (such as metformin), or a DPPIV-inhibitor (such as sitagliptin).

In various embodiments, a glucagon-like peptide 1 (GLP-1) analog is administered to a subject to treat a disease. Examples of GLP-1 analogs include but are not limited to exenatide and liraglutide.

In various embodiments, a dipeptidyl peptidase IV (DPPIV) inhibitor is administered to a subject to treat a disease. Examples of DPPIV inhibitors include but are not limited to sitagliptin, vildagliptin and saxagliptin.

In various embodiments, metformin is administered to a subject to treat a disease.

In various embodiments, a glinide is administered to a subject to treat a disease. Examples of glinides include but are not limited to repgalinide and nateglinide.

In various embodiments, a sulfonylurea is administered to a subject to treat a disease. Examples of sulfonylureas include but are not limited to gliclazide and glimepiride.

In various embodiments, an α-glucosidase inhibitor is administered to a subject to treat a disease. An example of an α-glucosidase inhibitor is acarbose.

In various embodiments, an insulin is administered to a subject to treat a disease. The term “insulin” by itself refers to any naturally occurring form of insulin as well as any derivatives and analogs thereof. Different types of insulin may vary in the onset, peak occurrence and duration of their effects. Examples of insulin that may be useful in the present invention include but are not limited to regular human insulin, intermediate acting regular human insulin (e.g., NPH human insulin), Zn-retarded insulin, short acting insulin analog and long acting insulin analog. Examples of Zn-retarded insulin include but are not limited to lente and ultralente. Examples of short-acting insulin analog include but are not limited to lispro, aspart and glulisine. Examples of long-acting insulin analog include but are not limited to glargine and levemir.

In various embodiments, one or more drug is combined with one or more treatment regimens such as diet, exercise and so on.

### Methods of Determining Treatment Efficacy

Additionally, therapeutic or prophylactic agents (i.e., drugs) suitable for administration to a particular subject can be identified by detecting one or more biomarkers in an effective amount from a sample obtained from a subject and exposing the subject-derived sample to a test compound that determines the amount of the one or more biomarker in the subject-derived sample. Accordingly, treatments or therapeutic regimens for use in subjects having a disease or subjects at risk for developing a disease can be selected based on the amounts of biomarkers in samples obtained from the subjects and compared to a reference value. Two or more treatments or therapeutic regimens can be evaluated in parallel to determine which treatment or therapeutic regimen would be the most efficacious for use in a subject to delay onset, or slow progression of a disease. In various embodiments, a recommendation is made on whether to initiate or continue treatment of a disease. Thus, the biomarker panels of the present invention can be used to determine the efficacy of treatment in a patient or subject.

Accordingly, in one aspect, the invention provides a method of assessing the efficacy of a first therapy on a subject comprising: taking a first measurement of a biomarker panel in a first sample from the subject; effecting the first therapy on the subject; taking a second measurement of the biomarker panel in a second sample from the subject; and making a comparison of the first measurement and the second measurement. In some embodiments, the method further comprises effecting a second therapy on the subject based on the comparison.

In some embodiments, the therapy comprises administering a disease-modulating drug to the subject. In these embodiments, changes in the levels of biomarkers between the first and second measurement allows a physician to either: a) keep the patient on a disease-modulating drug, as the changes in levels of certain biomarkers indicates the drug is working; b) keep the patient on the drug and adjust the dose; c) take the patient off the drug as efficacy is not present; and/or d) add an additional drug to the treatment, whether the patient is kept on the drug or not. Thus, effecting a second therapy in some embodiments comprises making a decision regarding the continued administration of the first disease-modulating drug.

In exemplary embodiments, the first therapy comprises administering a disease-modulating drug according to a first dosage regimen. In some embodiments, the first therapy comprises administering a combination of drugs according to a first dosage regimen. In exemplary embodiments, the combination comprises an insulin sensitizer drug. Thus, the methods of the invention can be used to test the efficacy of a combination of drugs, which can be modified for subsequent therapies according to differences in biomarker panel measurements.

A measurement of a biomarker panel will generally comprise the detection or observation of some characteristic (e.g., concentration (also referred to as a level)) of each member of the biomarker panel. A comparison of a first measurement and a second measurement will indicate a change, if any, in the measured characteristic for the biomarker of interest. A change as used herein may refer to any statistically relevant difference in the characteristic of a biomarker between a first measurement and a second measurement. A statistically relevant difference may be defined by the practitioner or by any art recognized method, and is generally defined herein. For example, a statistically relevant difference may be defined as a difference that surpasses a threshold defined by the practitioner. Thus, in various embodiments, making a comparison of the first measurement and the second measurement comprises determining the difference between the concentration of a biomarker in a first sample determined by the first measurement and the concentration of the biomarker in a second sample determined by the second measurement.

A change may refer to a single quantity, e.g., a 100% difference relative to a first measurement or may refer to a range, e.g., about 50% to about 100% difference or a ≧50% difference relative to a first measurement

A change may occur in either direction relative to a first measurement, i.e., the second measurement may be greater than or less than the first measurement. In some instances, there may be no change between measurements, and this absence of change may affect the therapeutic decision made by a practitioner in some embodiments.

Changes in the concentration of various combinations of biomarkers, such as those of a biomarker panel disclosed herein, will indicate to a practitioner a subject's responder status, i.e., whether or not a subject is a responder or nonresponder to a therapy. It should be appreciated that changes in biomarker concentrations can, in some cases, also indicate various degrees of response to a therapy. Thus, in some embodiments, a subject may be determined to be a strong responder, an intermediate responder or a weak responder. A subject associated with one of these response categories may optionally be given a different therapy compared to a subject associated with another. A practitioner can devise any number of response categories according to his or her needs.

Whether a subject is a responder or nonresponder to a therapy can be determined by the number and/or degree of changes observed in any combination of biomarkers of any biomarker panel disclosed herein. Identifying the responder status, which includes identifying nonresponder status, of a subject can aid the practitioner in choosing an appropriate therapy as discussed below.

One advantage of the biomarker panels of the invention is that they allow a practitioner to detect a response to a therapy, such as administration of a disease-modulating drug, within a short period of time, typically 1, 2, 3, 4, 5, 6 or 7 days, preferably within 1, 2, 3 or 4 days. Responder status can often be determined within 1 day after administration of the drug. Biomarker measurements made within 3 days after administration of the drug can be used to determine if changes in dosage are necessary. It may also be advantageous to detect a response to a therapy within 2, 3 or 4 weeks.

There are numerous ways of determining a subject's tendency to respond to a therapy. In various embodiments, a subject's responder status is based on a change observed for each biomarker of a biomarker panel or of a subset of the biomarker panel. In other words, if a biomarker panel comprises or consists of 9 biomarkers, a subject's responder status may be based on a change observed in 1, 2, 3, 4, 5, 6, 7, 8 or 9 biomarkers, in any combination.

In some embodiments, a change as defined above (e.g. an increase or a decrease, depending on the marker) in at least two of the markers allows calling a patient a “responder”, e.g. that the drug is beneficial to the patient. In alternative embodiments, a change in at least 3, 4, 5, 6, 7, 8 or 9 of the markers allows the continuation of the drug.

In some embodiments, measurements of biomarker concentrations may be combined with genotyping of the subject to determine a therapy. That is, by combining biomarker concentrations with a subject's genotype for expressing, for example, a particular member of the CYP superfamily, a practitioner can choose a therapy or dosage accordingly.

Once a practitioner has made a determination, based on the comparison of biomarker concentrations between a first and second measurement, as to whether a subject is a responder, nonresponder or a responder of a certain degree to a therapy (e.g. the administration of a disease-modulating drug), a practitioner may decide to effect a therapy based on this determination.

In some embodiments, the therapy comprises repeating or maintaining administration of a disease-modulating drug. A practitioner might choose this therapy, if, for example, a subject that is administered a disease-modulating drug according to a first dosage regimen is determined to be a responder based on a change or set of changes described herein. In some embodiments, if the concentrations of all of the biomarkers of a biomarker panel that are expressed in the macrophage/monocyte decrease (e.g., MCP-1, MMP-9, TNFα, IL6, p105, relA etc.), for example, at least 15% (or other appropriate value disclosed herein) compared to a first measurement, then the therapy comprises repeating or maintaining administration of a disease-modulating drug. In some embodiments, if the concentrations of all of the biomarkers of a biomarker panel decrease (except for biomarkers, such as an NFκB inhibitor, that tend to move in the opposite direction compared to others in indicating a response) compared to a first measurement, then the therapy comprises repeating or maintaining administration of a disease-modulating drug.

In some embodiments, the therapy comprises administering an additional drug to the subject, wherein the additional drug is different from a first administered drug. Other drugs useful in the present invention are described herein. An exemplary additional drug is a statin.

In some embodiments, the therapy comprises discontinuing administration of the disease-modulating drug. A practitioner might choose this therapy, if, for example, a subject that is administered a disease-modulating drug according to a first dosage regimen is determined to be a nonresponder, e.g., there is no change in one or more of the biomarker concentrations. A practitioner might also choose this therapy, if, for example, a subject is a weak responder. For instance, a practitioner might determine that the risks of administering a drug outweighs the benefits of the weak response. In some embodiments, if the concentration of one or more biomarkers do not increase or decrease in a manner indicative of response to a first therapy (such as administration of a disease-modulating drug) as described herein, then a second therapy comprises discontinuing the first therapy.

In some embodiments, the therapy comprises administering the disease-modulating according to a second dosage regimen. In these embodiments, the second dosage regimen will be different from the first dosage regimen associated with administration of the disease-modulating drug before measurement of a biomarker panel. In exemplary embodiments, the first dosage regimen comprises administering the disease-modulating drug at a first dose and the therapy comprises administering the disease-modulating drug at a second dose that is adjusted, depending on the degree of change in the expression of MCP-1 nucleic acid, MMP-9 nucleic acid or TNFα nucleic acid (or other nucleic acids of other panels), for example, or in the concentrations of some combination (such as all) of the biomarkers. In some embodiments, the therapy comprises administering disease-modulating drug according to an adjusted dosage regimen compared to a previous dosage regimen.

The biomarkers of the invention show a statistically significant difference between different responses to a disease-modulating drug. In various embodiments, diagnostic tests that use these biomarkers alone or in combination show a sensitivity and specificity of at least about 85%, at least about 90%, at least about 95%, at least about 98% and about 100%.

### Example

### Downregulation of the Proinflammatory State of Circulating Mononuclear Cells by Short Term Treatment with Pioglitazone in Patients with Type 2 Diabetes Mellitus

Presented herein are the short-term effects of an addition of pioglitazone (vs. placebo) to an existing effective oral anti-diabetic therapy with metformin and/or sulfonylurea on the proinflammatory activation of circulating mononuclear cells in well controlled patients with type 2 diabetes mellitus and elevated risk for atherosclerosis. For this purpose, we investigated the mRNA expression of the inhibitors to NF-κB (IκB-α and IκB-β) (26), p105 (precursor to the p50 subunit) and Rel-A (p65 subunit) as measures of the quantity of intranuclear NF-κB (27), and several proinflammatory mediators and markers that are known to be modulated by NF-κB, such as TNFα, IL-6, MIF, and MMP-9 (5, 12, 28) before and after four weeks of treatment.

This investigation was performed as a double-blind, placebo controlled, randomized multi-center study in patients with type 2 diabetes and established atherosclerosis. Inclusion criteria were an age between 20 and 80 years, an HbAlc<8.5%, an angiographically confirmed coronary artery disease and an activated chronic systemic inflammation (characterized by an increased hsCRP level≧1 mg/l). Patients had to be on any stable oral antidiabetic treatment with the exception of a thiazolidinedione for at least 3 months. Main exclusion criteria were: systemic inflammation of other origin, invasive cardiovascular intervention within the last 3 months, history of heart failure (NYHA I-IV), major hepatic or renal disease, and progressive fatal disease. The study was approved by the local ethical review board and the national regulatory agency, and all patients provided a written informed consent prior to study inclusion.

The patients were randomised by a telephone randomization procedure to either receive 45 mg pioglitazone or placebo in addition to their individual oral antidiabetic treatment for 4 weeks. Blood for the measurement of fasting glucose, MMP-9, and hsCRP was taken at baseline and after 3, 7, 10, 14 and 28 days of study treatment. Blood for assessment of the mRNA expression profile of circulating mononuclear cells as well as for assessment of circulating plasma levels of HbAlc, Insulin, Intact Proinsulin, Adiponectin, IL-6, sCD40L, P-Selectin, MIF, Angiotensin II, complement factor 3, and blood lipids were obtained at baseline and at the end of the study. Insulin resistance was calculated using the HOMA_{IR} score at baseline and study endpoint as published previously (29, 30).

HbAlc was measured by means of a HPLC method (Menarini, Neuss) and lipids were assessed by standard dry chemistry (Olympus, Hamburg, Germany). Immunoassays were applied to determine the plasma concentrations of insulin (CLIA, Invitreon, Cardiff, UK), intact proinsulin (CLIA, Invitreon, Cardiff, UK), adiponectin (RIA, Linco, St. Charles, Mo.), IL-6 (Elisa, IBL, Hamburg, Germany), MMP-9 (ELISA, R&D Systems, Wiesbaden, Germany), MCP-1 (ELISA, R&D Systems, Wiesbaden, Germany), sCD40L (ELISA, R&D Systems, Wiesbaden, Germany), P-selectin (R&D Systems, Wiesbaden, Germany), TNFα (ELISA, IBL, Hamburg, Germany), MIF (R&D Systems, Wiesbaden, Germany), Angiotensin II (ELISA, DRG-Diagnostics, Marburg, Germany), and complement factor C3 (ELISA, BioCat, Heidelberg, Germany).

Isolation of MNC from whole blood was performed as a density gradient centrifugation by means of the ACCUSPIN System-HISTOPAQUE-1077 (Sigma-Aldrich Chemie GmbH, Steinheim, Germany). The isolation of macrophages and monocytes from the collected cells was performed by MACS magnetic cell sorting with CD14 MicroBeads (human) (Miltenyi Biotec GmbH, Bergisch Gladbach, Germany). The CD14 positive (CD14+) cells (macrophages and monocytes) were first magnetically labeled with MACS CD14 MicroBeads. The cell suspension was loaded on a MS MACS Column which was placed in the magnetic field of a MiniMACS Separator. The magnetically labeled CD14+ cells were retained in the column and separated from the unlabelled cell fraction.

The mRNA isolation from macrophages and monocytes was performed with the High Pure RNA Isolation Kit (Roche Applied Science, Penzberg, Germany). The cells were first lysed and the intact and undegraded RNA was adsorbed to a glass fibre fleece. Simultaneously, RNAses were inactivated. Furthermore, residues of contaminating DNA were digested and the RNA was purified from salts, proteins and other impurities. Purity of the isolated mRNA was assessed by real-time PCR (LightCycler II, Roche Diagnostics, Mannheim, Germany). The transcription of mRNA in cDNA was performed on a thermocycler (Biozym Diagnostik GmbH, Oldendorf, Germany) according to a standardized protocol (‘Transcriptor First Strand cDNA Synthesis Kit’, Instruction Manual Version 1, 2004, Roche Applied Science, Penzberg, Germany).

Sequence specific primers were designed by TIB MOLBIOL (Syntheselabor GmbH, Berlin, Germany) to amplify the gene sequences of Rel-A, p105, IκB-α, IκB-β and IL-6. The primers for TNF-α were taken from “Rapid Cycle Real-Time PCR Methods and Applications Quantification” (31). The primers for MIF and MMP-9 were reproduced from previous reports (12, 32). A list of the primers used for the quantification experiments, the primer-specific PCR protocols and the specific amplification product melting points are provided in Table 1. An additional agarose gel electrophoresis assay was performed to verify the correct length of the amplification products.

TABLE 1
Primer composition and PCR Protocols

| | | | | | Product |
|---|---|---|---|---|---|
| | | Annealing | | | melting |
| | Forward Primer | temperature | Annealing | Elongation | temperature |
| Gene | Reverse Primer | [° C.] | time [s] | time [s] | [° C.] |
| Rel-A | CAGTACCTGCCAGATACAGACGA | 63 | 10 | 7 | 88 |
| | GGGAAGGCACAGCAATG | | | | |
| P105 | TGATGATTTACTAGCACAAGGAGACAT | 65 | 10 | 9 | 85 |
| | TGTACCCCCAGAGACCTCATAG | | | | |
| IκB-α | CTGATGTCAATGCTCAGGAGCC | 68 | 10 | 11 | 89 |
| | TGTGTCATAGCTCTCCTCATCCTCAC | | | | |
| IκB-β | CTGAAAACTACGAGGGCCA | 64 | 5 | 8 | 91 |
| | CCTCCACTGCCAAATGAAG | | | | |
| TNF-α | CCCAGGGACCTCTCTCTAATC | 64 | 10 | 8 | 87 |
| | ATGGGCTACAGGCTTGTCACT | | | | |
| IL-6 | CCCATGCAGGCACTTACTAC | 63 | 5 | 4 | 86 |
| | ACGTCTTCTTGAACCTCAGAACA | | | | |
| MIF | CGGACAGGGTCTACATCAA | 63 | 5 | 4 | 84 |
| | CTTAGGCGAAGGTGGAGTT | | | | |
| MMP-9 | CCCATTTCGACGATGACGAGTTGTG | 64 | 10 | 13 | 92 |
| | GGAGTAGGATTGGCCTTGGAAGATG | | | | |

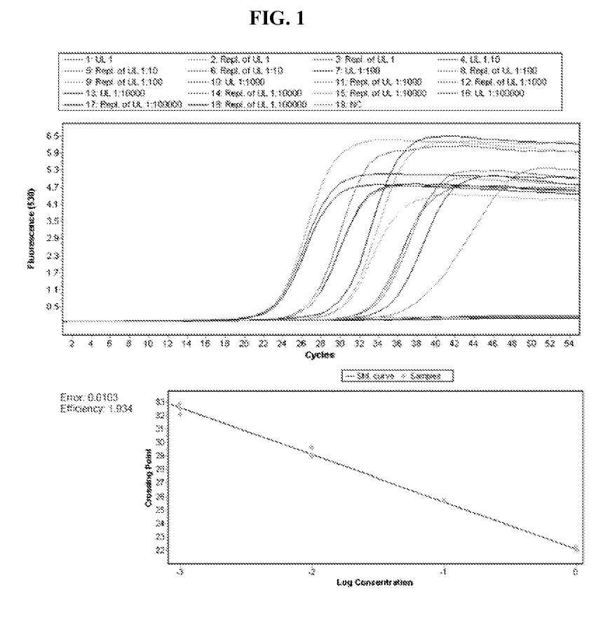

The RNA quantification in this study, was performed by means of a calibrator-normalized relative quantification method based on the LightCycler II system (Roche Diagnostics, Mannheim, Germany), where quantification of a target and a reference gene is a function of PCR efficiency and the sample crossing point. The sample crossing point is the amplification cycle during an amplification assay, at which the fluorescence of a probe rises above background fluorescence. This occurs usually at the second derivative maximum (fastest change in fluorescence). The calibrator, a positive sample for the investigated gene product must have a constant ratio of target gene expression to reference gene expression. In these experiments, β-actin is the most abundant protein in eukaryotic cells with constant expression (33). The results of the calibrator-normalized quantification are expressed as the target/reference ratio of each sample divided by the target/reference ratio of the calibrator. The principle of this method is the determination of the relative amount of the target gene and the reference gene for each sample and for the calibrator. Quantification results are provided as Normalized Ratio (target marker concentration [sample]/reference concentration [sample])/(target marker concentration [calibrator]/reference concentration [calibrator]). For each RNA marker investigated in this study, a standard curve was created, to be able to compare the unknown values of the patient samples to a standard value of a calibrator, and to calculate the ratios relevant for quantification of the levels of mRNA expression. An example is shown in FIG. 1. All experiments were performed in triple replications.

Data are presented as arithmetic mean±standard deviation (SD) for continuous variables or mean±SEM for percent changes from baseline and as the number/proportion of patients for categorical variables. For the changes from baseline of the efficacy parameters one-sided p-values for within-group treatment differences were calculated, using the paired t-test procedure. Wilcoxon's two sample test was used to calculate one-sided p-values for between-group treatment differences. No transformations were applied to the secondary efficacy parameters. All inferential statistical analyses were performed in an exploratory sense, and all p-values<0.05 were interpreted as statistically significant.

In total, 63 patients matching the inclusion and exclusion criteria could be included into this investigation (11 women, 52 men; age: 65.6±6.9 years (range: 45-77 years); disease duration: 6.6±9.6 years (range: 0-58 years), HbAlc: 6.7±0.6%; BMI: 30.7±4.2 kg/m²). All but one patient had a known prevalence of cardiovascular disease (98.4%), and 59 suffered from hypertension (93.6%). A total of 9 patients were current smokers (14.3%) and another 37 reported smoking in the past (58.7%). The study drugs were well tolerated and all but one patient in the pioglitazone arm completed the study per protocol. This patient dropped out based on a personal decision after realizing a mismatch between personal schedules and study visit requirements.

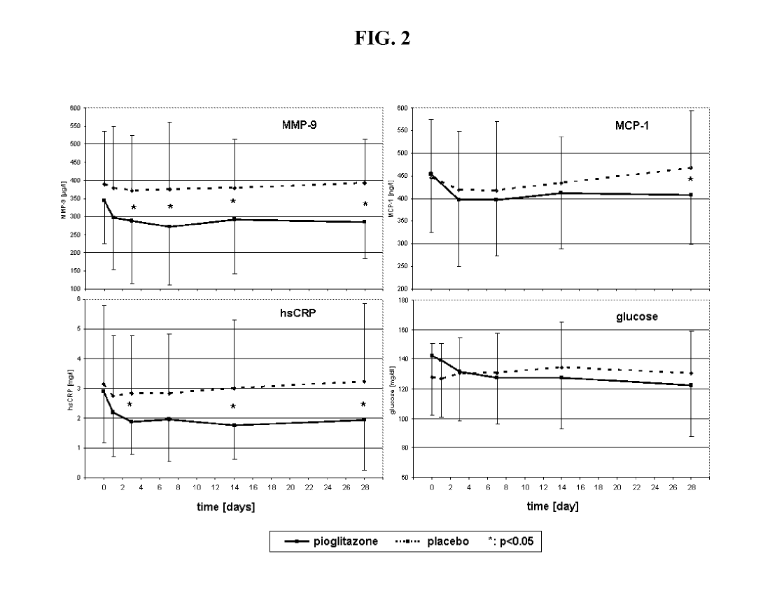

The change in fasting glucose concentrations and in the inflammatory cardiovascular risk markers MMP-9, MCP-1, and hsCRP during the observation period is provided in FIG. 2. While a slight but non-significant decrease in fasting glucose could be observed with pioglitazone, the same group showed a fast and significant decrease in MMP-9 and hsCRP that was not seen in the placebo group. There was no significant change from baseline to endpoint in HbAlc in this well controlled patient population in any of the two treatment groups, but a significant improvement in insulin resistance and the metabolic syndrome as indicated by a decrease in the HOMA_{IR} score, a decrease in intact proinsulin concentrations, and an increase in adiponectin values was observed in patients treated with pioglitazone (p<0.001 vs. placebo at endpoint in all cases).

The mean absolute values for all determined plasma proteins and the other observation parameters at baseline and endpoint is provided in Table 2. There were significant improvements in many of these markers after 4 weeks of pioglitazone treatment, indicating an overall reduction of the inflammatory situation in the circulating blood, an improvement in endothelial and thrombocyte function and an improvement in the metabolic risk situation. The differences between from baseline to endpoint in the pioglitazone group and between the treatment groups at endpoint reached the level of statistical significance for many of the observation parameters.

TABLE 2
Clinical and biochemical observation parameters at
baseline and after 4 weeks in both treatment arms

| | pioglitazone | | placebo | |
|---|---|---|---|---|
| | Baseline | Endpoint | Baseline | Endpoint |
| HbA1c [%] | 7.0 ± 1.1 | 6.8 ± 0.9 | 6.7 ± 0.6 | 6.6 ± 0.7 |
| BMI [kg/m²] | 31.0 ± 4.3 | 31.4 ± 4.5* | 30.5 ± 4.1 | 30.4 ± 4.2 |
| Systolic blood pressure [mmHg] | 144 ± 15 | 137 ± 18* | 141 ± 19 | 139 ± 20 |
| Diastolic blood pressure [mmHg] | 83 ± 11 | 80 ± 11 | 78 ± 11 | 79 ± 9 |
| Waist/hip ratio | 1.00 ± 0.05 | 1.00 ± 0.07 | 1.00 ± 0.06 | 1.00 ± 0.06 |
| Glucose [mg/dl] | 142 ± 40 | 122 ± 35*** | 128 ± 23 | 131 ± 29 |
| Insulin [μU/ml] | 17 ± 10 | 12 ± 7*** | 18 ± 10 | 18 ± 11⁺ |
| HOMA_{IR} | 5.9 ± 4.4 | 3.9 ± 2.6*** | 6.0 ± 3.5 | 6.4 ± 4.5⁺ |
| Adiponectin [mg/dl] | 8.7 ± 3.5 | 22.1 ± 9.1*** | 8.3 ± 4.7 | 8.2 ± 4.2⁺⁺⁺ |
| Intact proinsulin [pmol/l] | 30 ± 37 | 19 ± 17* | 24 ± 19 | 24 ± 20 |
| hsCRP [mg/l] | 2.9 ± 1.7 | 1.9 ± 1.7** | 3.2 ± 2.6 | 3.2 ± 2.6⁺ |
| MMP-9 [μg/l] | 344 ± 118 | 284 ± 101** | 388 ± 147 | 391 ± 121⁺⁺⁺ |
| MCP-1 [μg/l] | 454 ± 130 | 406 ± 106* | 446 ± 129 | 468 ± 127⁺ |
| Total Cholesterol [mmol/l] | 4.63 ± 0.99 | 4.78 ± 1.09 | 4.60 ± 1.10 | 4.61 ± 1.00 |
| LDL cholesterol [mmol/l] | 2.46 ± 0.80 | 2.52 ± 0.76 | 2.21 ± 0.93 | 2.21 ± 0.90 |
| HDL cholesterol [mmol/l] | 1.18 ± 0.24 | 1.26 ± 0.26** | 1.12 ± 0.21 | 1.13 ± 0.19⁺ |
| Triglycerides [mmol/l] | 1.84 ± 1.14 | 1.72 ± 0.99 | 2.41 ± 2.22 | 2.53 ± 1.73⁺ |
| sICAM [μg/l] | 326 ± 90 | 319 ± 86 | 304 ± 56 | 310 ± 60 |
| sVCAM [μg/l] | 915 ± 409 | 943 ± 452 | 797 ± 193 | 807 ± 220 |
| sCD40L [μg/l] | 1.6 ± 1.8 | 1.1 ± 1.1 | 1.4 ± 1.2 | 1.0 ± 0.9 |
| P-seclectin [μg/l] | 95 ± 20 | 93 ± 22 | 96 ± 26 | 98 ± 26 |
| IL-6 [ng/l] | 3.3 ± 0.5 | 3.2 ± 0.1 | 3.3 ± 0.3 | 3.3 ± 0.4 |
| Angiotensin II [μg/l] | 10.2 ± 9.2 | 8.0 ± 8.8* | 9.2 ± 8.2 | 10.1 ± 10.1 |
| Complement factor C3 [g/l] | 1.6 ± 0.4 | 1.5 ± 0.3 | 1.6 ± 0.4 | 1.7 ± 0.5 |
| MIF [μg/l] | 10.4 ± 5.3 | 9.6 ± 3.5 | 10.1 ± 4.1 | 10.3 ± 4.3 |

Within group comparison:
*p < 0.05;
**p < 0.01; ;
***p < 0.001 vs. baseline
Between groups for changes from baseline:
⁺p < 0.05;
⁺⁺p < 0.01; ;
⁺⁺⁺p < 0.001

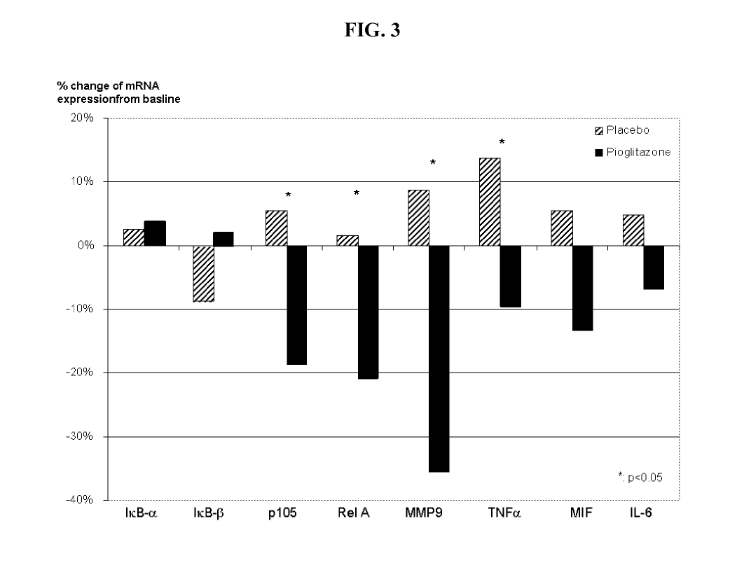

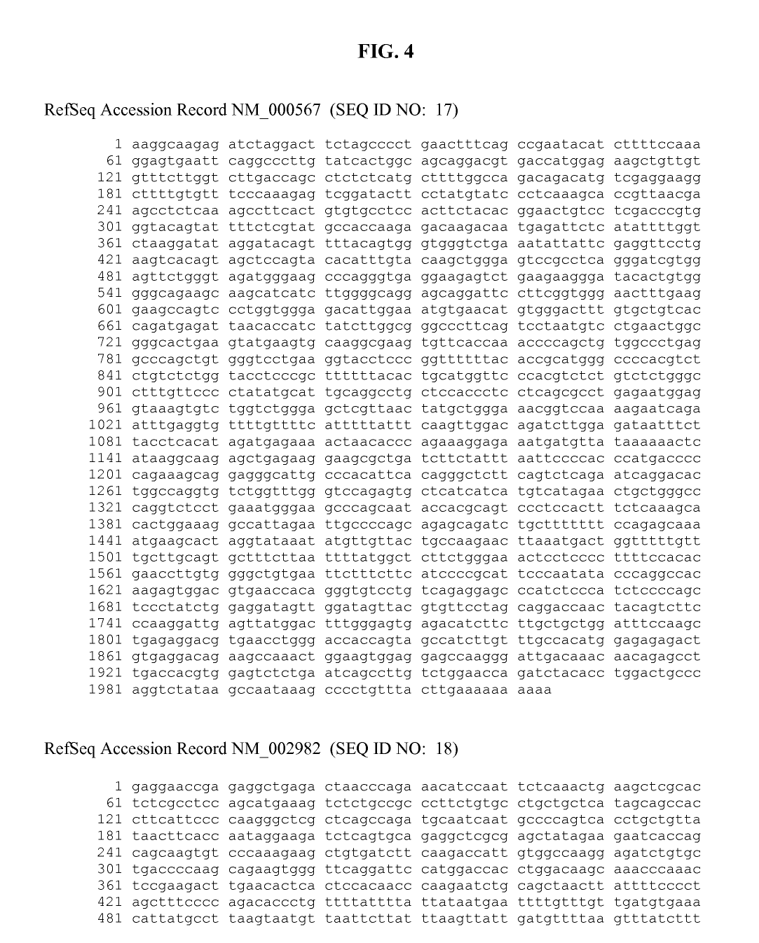

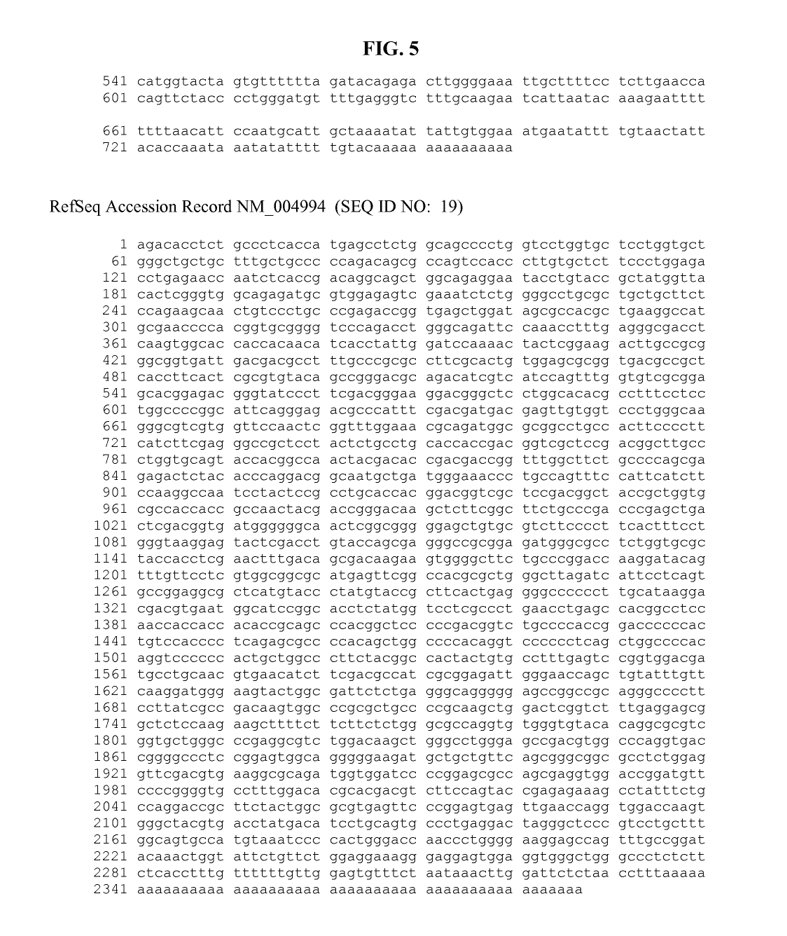

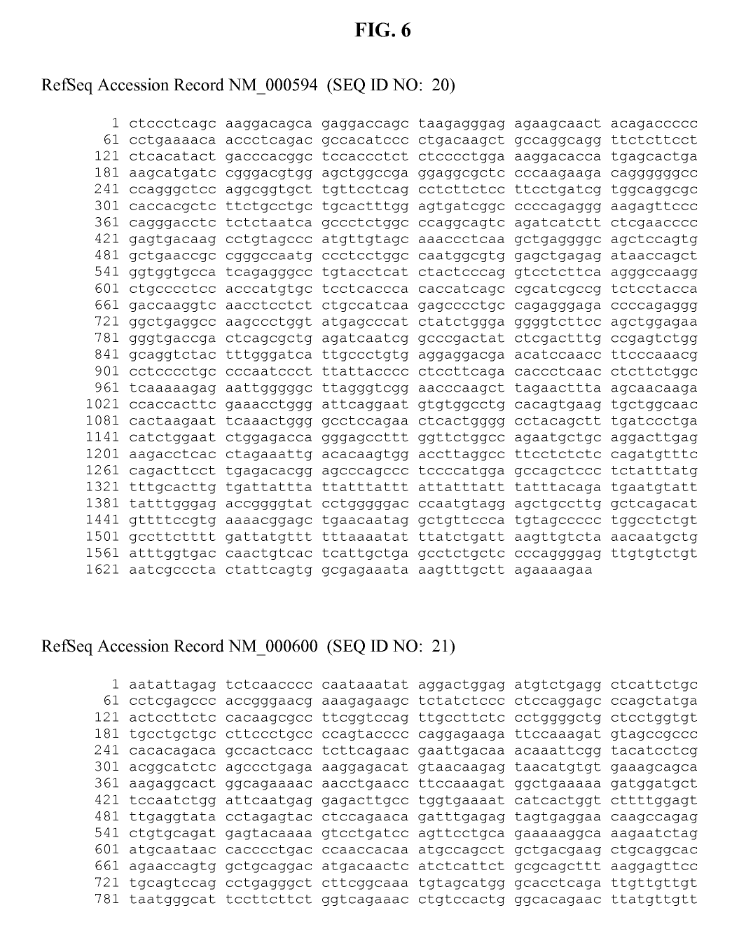

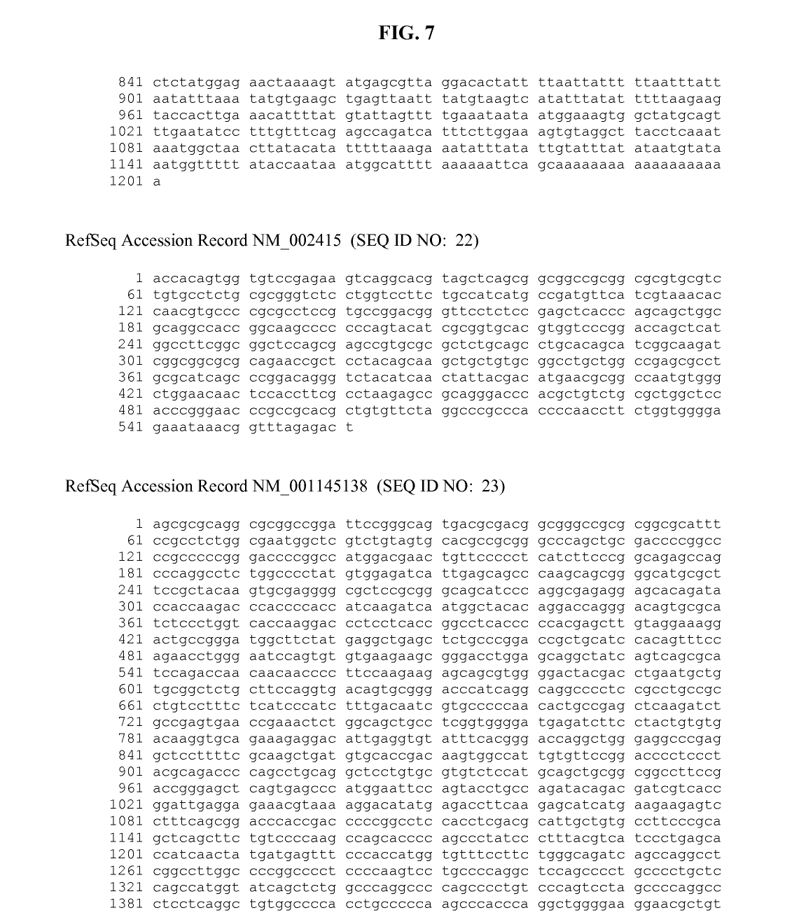

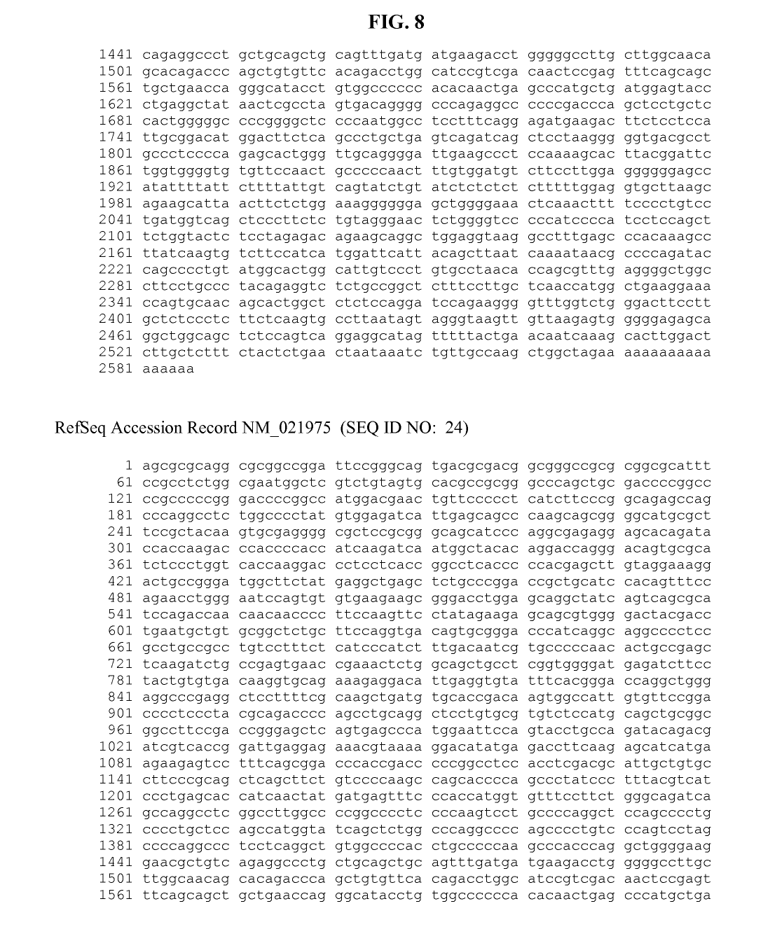

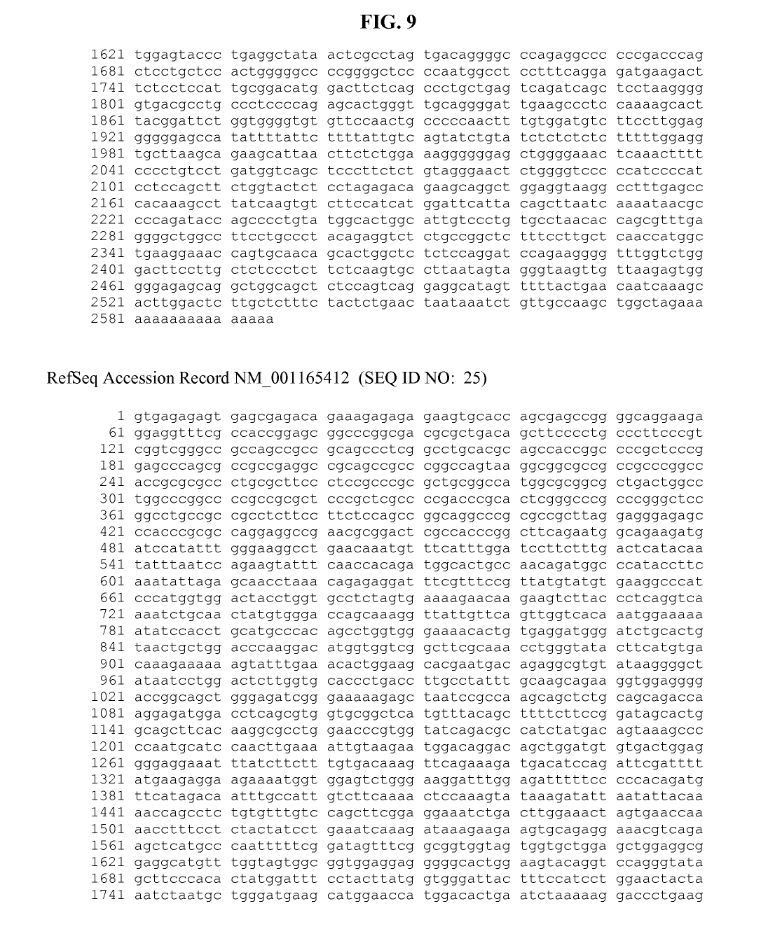

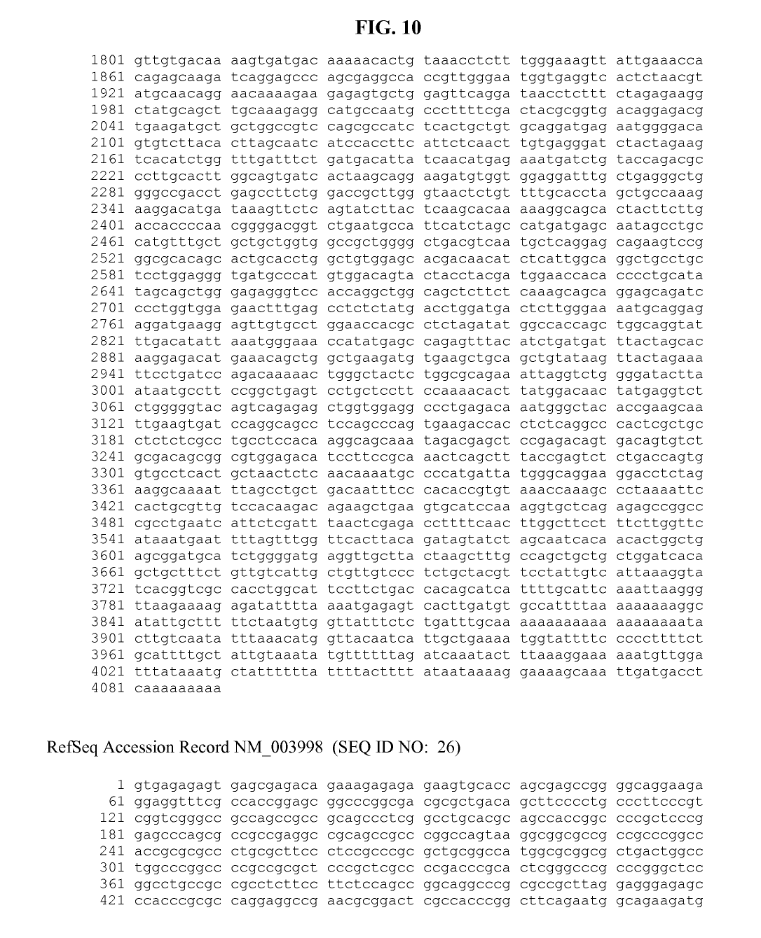

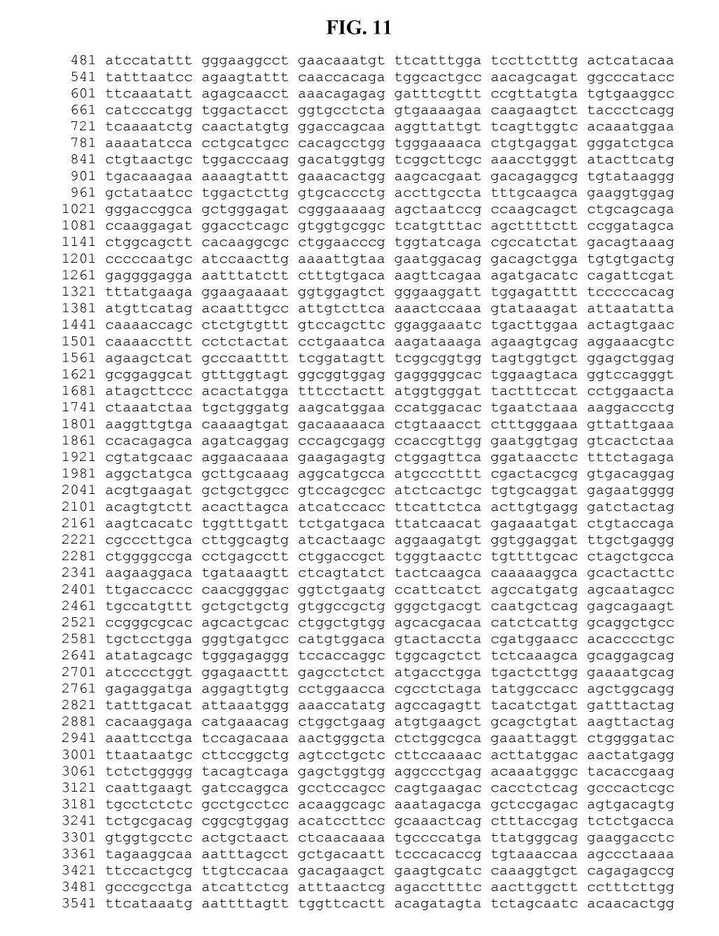

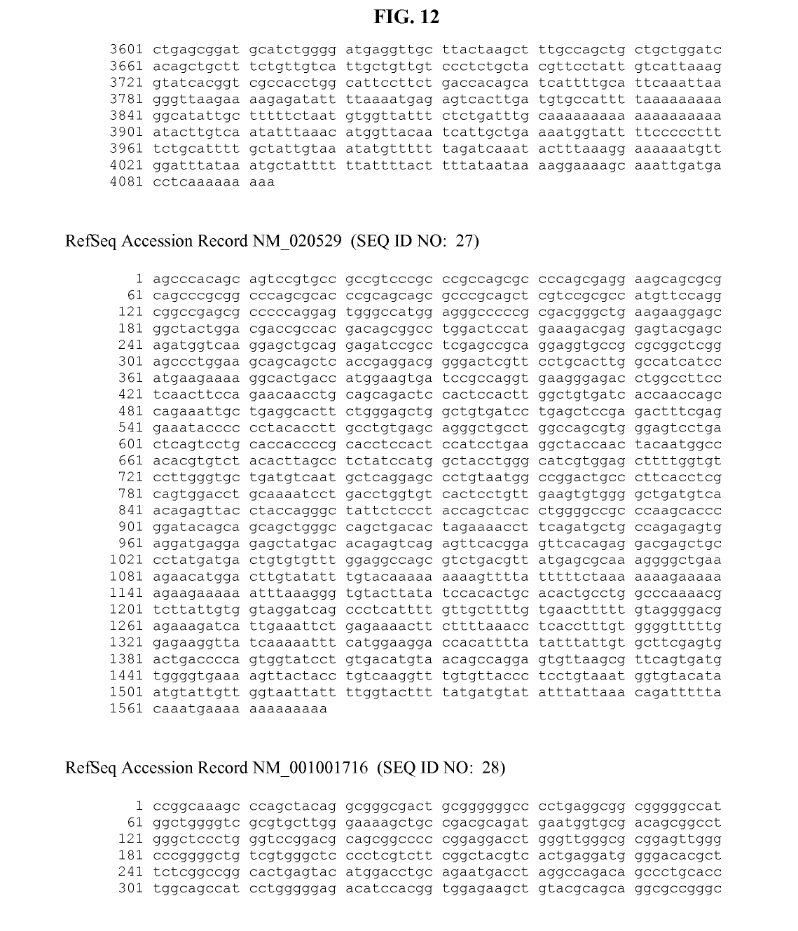

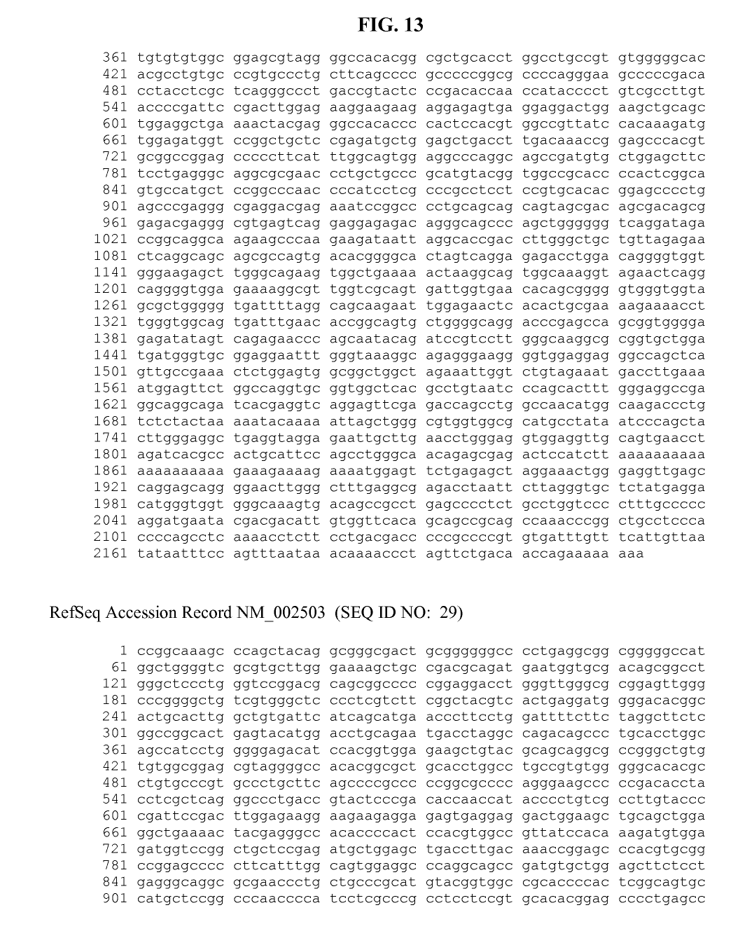

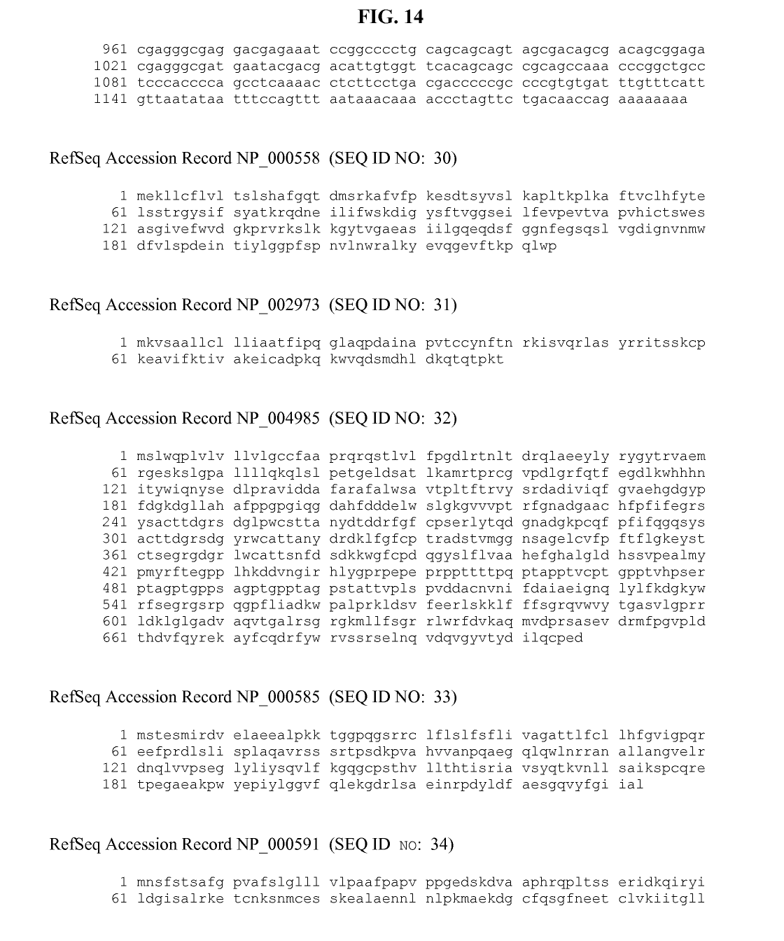

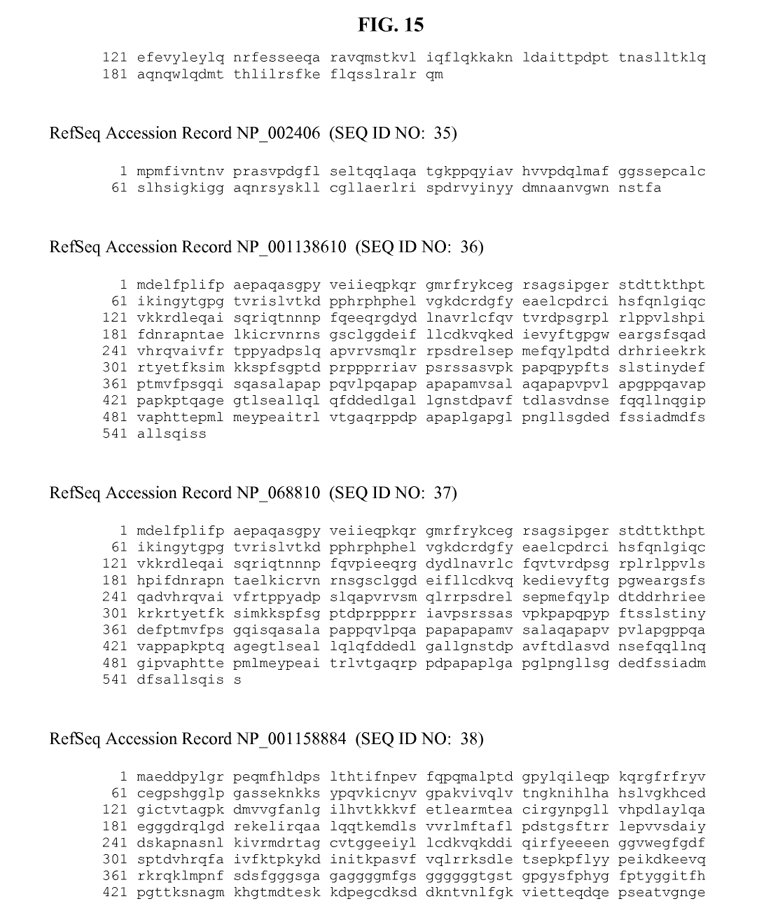

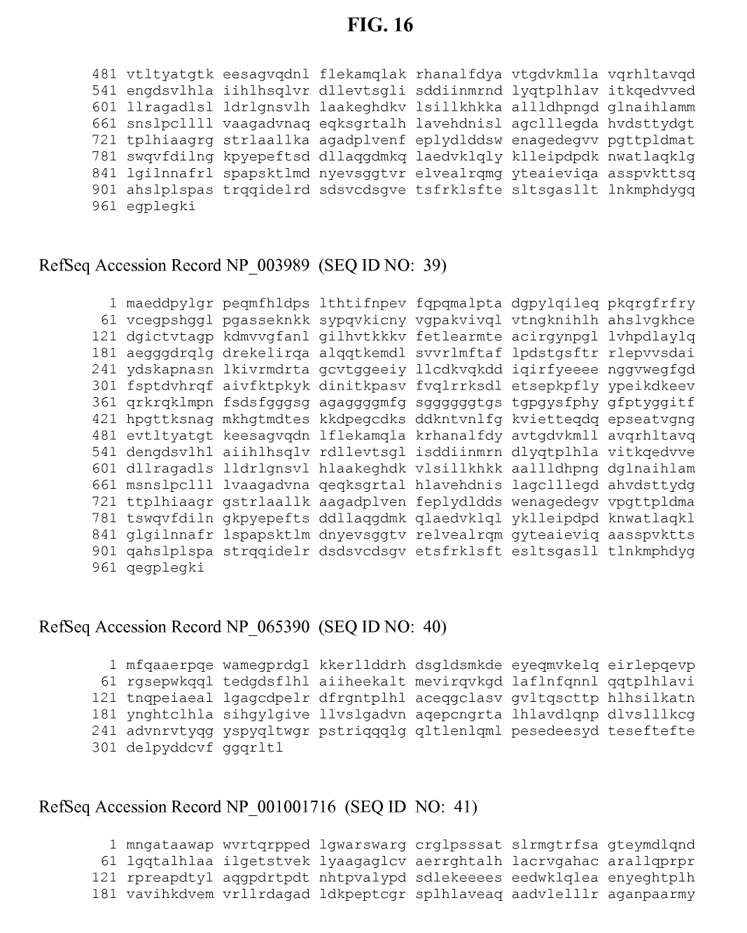

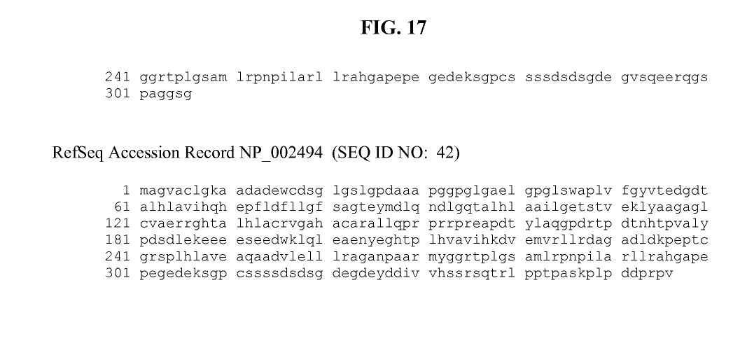

The quantification of the mRNA expression of the investigated proinflammatory cytokines in relation to a calibrator gene (β-actin) is provided in Table 3. The relative expression of all proinflammatory markers increased in patients treated with placebo and decreased in patients on additional pioglitazone therapy, while the expression of the inhibitory markers changed inversely. The difference between the groups at endpoint was statistically significant for MMP-9, TNFα, RelA and p105. The percent changes in the mRNA expression of the observed biomarkers for both treatments is provided in FIG. 3. The changes in MMP-9 mRNA expression were reflected by the corresponding protein concentrations. The overall expression pattern demonstrated a comprehensive decrease in the inflammatory state of the circulating monocytes during pioglitazone therapy, while a further increase of proinflammatory mRNA expression was observed with placebo.

TABLE 3
mRNA expression of NFκB and NFκB-modulated cytokines in circulating
peripheral mononuclear cells at baseline and after 4 weeks of therapy with
pioglitazone or placebo (reference gene: β-actin)

| | Pioglitazone | | placebo | |
|---|---|---|---|---|
| | Baseline | Endpoint | Baseline | Endpoint |
| p105 | 1.63 ± 0.80 | 1.33 ± 0.46* | 1.35 ± 0.66 | 1.42 ± 0.91⁺ |
| (p50 subunit | | | | |
| of NF-κB) | | | | |
| RelA | 1.20 ± 0.74 | 0.95 ± 0.44* | 1.05 ± 0.42 | 1.07 ± 0.45⁺ |
| (p65 subunit | | | | |
| of NF-κB) | | | | |
| IκB-α | 1.28 ± 1.15 | 1.30 ± 1.14 | 1.13 ± 0.97 | 1.04 ± 0.75 |
| IκB-β | 3.17 ± 2.05 | 3.30 ± 2.41 | 2.78 ± 1.63 | 2.86 ± 1.62 |
| MMP-9 | 2.29 ± 2.68 | 1.48 ± 1.18 | 1.56 ± 2.02 | 1.69 ± 1.70⁺ |
| TNFα | 1.88 ± 1.20 | 1.70 ± 0.93 | 1.81 ± 0.94 | 2.02 ± 1.28⁺ |
| MIF | 0.98 ± 0.40 | 0.84 ± 0.31 | 0.83 ± 0.35 | 0.88 ± 0.52 |
| IL-6 | 1.13 ± 0.52 | 1.05 ± 0.46 | 1.09 ± 0.57 | 1.14 ± 0.69 |

*p < 0.05 (vs. baseline);
⁺p < 0.05 (between the groups for change from baseline)

The participants of our study had a good glycemic control by means of metformin and/or sulfonylurea drugs. The addition of pioglitazone induced a rapid reduction in the inflammatory expression state of circulating monocyte/macrophages, which went in parallel with a reduction of the plasma concentrations of corresponding plasma proteins and additional biomarkers for chronic inflammation in diabetic patients, which could not be observed in the placebo arm. Although the differences in mRNA marker expression between the two observation groups did not reach the level of statistical significance for all markers, the general expression pattern uniformly points into the direction of a comprehensive down-regulation of macrophage activation by pioglitazone. The expression of the NF-κB-related proteins (RelA and p105) and of NF-κB-regulated proteins (TNF-α, MIF, MMP-9) was reduced, while the expression of the inhibitors to NF-κB (IκB-α and IκB-β) apparently increased. These anti-inflammatory effects preceded any possible effects on glycemia by pioglitazone, and the 4 week observation period was too short to observe a significant change in HbAlc in this trial. There were, however, several signs for an improvement in insulin resistance, β-cell function, endothelial function, thrombocyte function and the metabolic syndrome as indicated by appropriate changes in the corresponding laboratory biomarkers.

The present example provides insight into the underlying cellular mechanisms of the short-term glucose-independent clinical effects of pioglitazone and rosiglitazone on endothelial and vascular function that were published recently in non-diabetic subjects and patients with type 2 diabetes. Hetzel and Coworkers demonstrated that a three-week treatment with rosiglitazone did not change blood glucose or lipid levels of healthy subjects, but increased flow-mediated, endothelium-dependent vasodilatation starting already within the first day, which was paralleled by a rapid reduction of pro-inflammatory and pro-thrombotic biomarkers. They suggested a direct effect of PPARγ activation on endothelial function and inflammation, independent from metabolic action (24).

Another group performed a randomized, placebo-controlled, double-blind crossover trial in 20 patients with type 2 diabetes on effective other oral anti-diabetic medication, to investigate the effect of treatment with 30 mg of pioglitazone on shear-stress induced flow-mediated vasodilatation. After 4 weeks, they found an amelioration of endothelial function in conduit arteries irrespective of significant beneficial changes in the plasma levels of insulin, free fatty acids, adiponectin, or hsCRP (25). Also, treatment with pioglitazone improved cutaneous microcirculation and endothelial function independent from glycemic control when compared with glimepiride (34).

The present results contribute to a better understanding of these effects on a molecular basis. Both abdominal fat and insulin resistance contribute to vascular disease, especially in obese patients. In particular, visceral fat contributes to inflammation and endothelial dysfunction through secretion of adipokines, like TNFα or IL-6, which are secreted by the lipid tissue after macrophage recruitment (through monocyte chemoattractant protein 1 (MCP-1)) (11). Pioglitazone has been demonstrated to decrease a variety of these adipokines in several clinical and experimental studies (19, 35, 36). The decrease of several plasma proteins such as IL-6, TNFα, Angiotensin II, and the increase in adiponectin in our current experiment is also in line with these findings. Clinical studies addressing these effects have only been able to investigate the fat tissue as a whole, i.e. including all different cellular subfractions. Fontana et al. were able to demonstrate by assessment of arteriovenous concentration differences with samples obtained from the portal vein that visceral fat is a clinically important site of IL-6 secretion, thus contributing to systemic inflammation (37). Our results suggest that at least part of the observed increase in proinflammatory cytokines may have derived from previously circulating mononuclear cells that had penetrated into the adipose tissue.

One possible explanation for the observed results with pioglitazone could be a direct effect of the thiazolidinedione on mononuclear cells. It has been shown that PPARγ has distinct functions in different cell types in the white adipose tissue, such that pioglitazone reduces macrophage infiltration by inducing apoptotic cell death specifically in macrophages through PPARγ activation (38). Since the macrophages recruited into the fat tissue are a major source of cytokines and proteins that are known to maintain systemic inflammation (11), a change in their inflammatory activity may be reflected by a down-regulation of proinflammatory mRNA expression in the circulating mononuclear cells.

Another contributor may also be an indirect effect of pioglitazone via modification of adipokine secretion derived from differentiating preadipocytes and other components of the lipid tissue.

The present example has a number of clinical implications. The observed pleiotropic effects of pioglitazone occur fast and mainly independent of the metabolic effects of the drug. This finding may support an earlier and more frequent use of this drug in patients who are still well controlled with other “classical” anti-diabetic drugs, but are at elevated risk for macrovascular disease. We have been able to demonstrate a significant decrease in surrogate markers for systemic inflammation and cardiovascular risk, including Intima-Media-Thickness, insulin resistance, endothelial function, hsCRP, MMP-9, or MCP-1 with pioglitazone, while other anti-diabetic drugs resulting in an equal improvement of metabolic control had no such effects in diabetic patients (18, 19, 35). Pioglitazone, when given by us in comparison or in addition to simvastatin had an independent synergistic impact on the cardiovascular risk of patients with normoglycemic vascular insulin resistance (36, 39). These clinical findings are in good agreement with our current observation of an overall down-regulation of the inflammatory state of circulating monocyte/macrophages by pioglitazone independent from glycemic control.

The results of our study indicate that pioglitazone when given in addition to an effective antidiabetic treatment with metformin and/or sulfonylurea induced an overall decrease in plasma adipokines and in the inflammatory state of circulating mononuclear cells in patients with well controlled type 2 diabetes mellitus, while a further deterioration was observed with placebo. These effects occurred independently from glycemic control and already after short treatment duration. Our findings are helpful to understand the mechanism and nature of the multiple anti-atherosclerotic and anti-thrombotic effects that have been reported in recent controlled clinical investigations and outcome trials comparing thiazolidinediones with other anti-diabetic drugs.

The articles “a,” “an” and “the” as used herein do not exclude a plural number of the referent, unless context clearly dictates otherwise. The conjunction “or” is not mutually exclusive, unless context clearly dictates otherwise. The term “include” is used to refer to non-limiting examples.

All references, publications, patent applications, issued patents, accession records and databases cited herein, including in any appendices, are incorporated by reference in their entirety for all purposes.

### CITATIONS

1. Stevens J, Cai J, Pamuk E R, Williamson D F, Thun M J, Wood J L. The effect of age on the association between body-mass index and mortality. N Engl J. Med. 1998; 338:1-7.
2. Calle E E, Thun M J, Petrelli J M, Rodriguez C, Heath Jr C W. Body-mass index and mortality in a prospective cohort of US adults. N Engl J Med. 1999; 341:1097-1105.
3. Dandona P, Mohanty P, Ghanim H et al. The suppressive effect of dietary restriction and weight loss in the obese on the generation of oxygenspecies ba leukocytes, lipid peroxidation, and protein carbonylation. J Clin Endocrinol Metab. 2001; 86:355-62.
4. Dandona P, Weinstock R, Thusu K, Abdel-Rahman E, Aljada A, Wadden T, Tumor necrosis factor-alpha in sera of obese patients: fall with weight loss. J Clin. Endocrinol Metab. 1998; 83:2907-10.
5. Bastard J P, Jardel C, Delatore J, Hainque B, Bruckert E, Oberlin F. Evidence for a link between adipose tissue interleukin-6 content and serum C-reactive protein concentrations in obese subjects. Circulation. 1999; 99:2221-22
6. Teramoto S, Yamamoto H, Ouchi Y. Increased C-reactive protein and increased plasma interleukin-6 may synergistically affect the progression of coronary atherosclerosis in obstructive sleep apnea syndrome. Circulation. 2003; 107:E40-40.
7. Samad F, Loskutoff D J. Tissue distribution and regulation of plasminogen activator inhibitor-1 in obese mice. Mol Med. 1996; 2:586-92.
8. Laimer M, Ebenbichler C F, Kaser S et al. Markers of chronic inflammation and obesity: a prospective study on the reversibility of this association in middle-aged women undergoing weight loss by surgical intervention. Int J Obes Relat Metab Disord. 2002; 26:659-62.
9. Fain J N, Madan A K, Hiler M L, Cheema P, Bahouth S W. Comparison of the release of adipokines by adipose tissue, adipose tissue matrix, and adipocytes from visceral and subcutaneous abdominal adipose tissues of obese humans. Endocrinology. 2004; 145:2273-82.
10. Bastard J P, Maachi M, Lagathu C et al. Recent advances in the relationship between obesity, inflammation, and insulin resistance. Eur Cytokine Netw. 2006; 17:4-12.
11. VanGaal L F, Mertens I, DeBlock C E. Mechanisms linking obesity with cardiovascular disease. Nature. 2006; 444:875-80.
12. Ghanim H, Aljada A, Hofineyer D, Syed T, Mohanty P, Dandona P. Circulating mononuclear cells in the obese are in a proinflammatory state. Circulation. 2004; 110:1564-71.
13. Dandona P, Aljada A, Ghanim H et al. Increased plasma concentration of macrophage migration inhibitory factor (MIF) and MIF mRNA in mononuclear cells in the obese and the suppressive action of Metformin. J Clin Endocrinol Metab. 2006; 89:5043-7.
14. Simpson S H, Majumdar S R, Tsuyuki R T, Eurich D T, Johnson J A. Dose-response relation between sulfonylurea drugs and mortality in type 2 diabetes mellitus: a population-based cohort study. CMAJ. 2006; 17:169-74.
15. Turner R C. The U.K. Prospective Diabetes Study. A review. Diabetes Care. 1998; 21(Suppl 3):C35-C38.
16. Ghanim H, Garg R, Aljada A et al. Suppression of nuclear factor-kappaβ and stimulation of inhibitor kappaβ by troglitazone: evidence for an anti-inflamatory effect and a potential antiatherosclerotic effect in the obese. J Clin Endocrinol Metab. 2001; 86:1306-12.
17. Masubuchi Y. Metabolic and non-metabolic factors determining troglitazone hepatotoxicity: a review. Drug Metab Pharmacokinet. 2006; 21:347-56.
18. Langenfeld M R, Forst T, Hohberg C et al. Pioglitazone decreases carotid intima-media thickness independently of glycemic control in patients with type 2 diabetes mellitus: results from a controlled randomized study. Circulation. 2005; 111:2525-31.
19. Pfützner A, Marx N, Lübben G et al. Improvement of cardiovascular risk markers by pioglitazone is independent from glycemic control: results from the pioneer study. J Am Coll Cardiol. 2005; 45:1925-31.
20. Mazzone T, Meyer P M, Feinstein S B et al. Effect of pioglitazone compared with glimepiride on carotid intima-media thickness in type 2 diabetes: a randomized trial. JAMA. 2006; 296:2572-81.
21. Dormandy J A, Charbonnel B, Eckland D J et al. Secondary prevention of macrovascular events in patients with type 2 diabetes in the PROactive Study (PROspective pioglitAzone Clinical Trial In macroVascular Events): a randomised controlled trial. Lancet. 2005; 366:1279-89.
22. Erdmann E, Dormandy J A, Charbonnel B, Massi-Benedetti M, Moules I K, Skene AM; PROactive Investigators. The effect of pioglitazone on recurrent myocardial infarction in 2,445 patients with type 2 diabetes and previous myocardial infarction: results from the PROactive (PROactive 05) Study. J Am Coll Cardiol. 2007; 49:1772-80.
23. Wilcox R, Bousser M G, Betteridge D J et al. Effects of Pioglitazone in Patients With Type 2 Diabetes With or Without Previous Stroke. Results From PROactive (PROspective pioglitAzone Clinical Trial In macroVascular Events 04). Stroke. 2007; 38:865-73.
24. Hetzel J, Balletshofer B, Rittig K et al. Rapid effects of rosiglitazone treatment on endothelial function and inflammatory biomarkers. Arterioscler Thromb Vasc Biol. 2005; 25:1804-9.
25. Martens F M, Visseren F L, de Koning E J, Rabelink T J. Short-term pioglitazone treatment improves vascular function irrespective of metabolic changes in patients with type 2 diabetes. J Cardiovasc Pharmacol. 2005; 46:773-8.
26. Vincent H K, Powers S K, Stewart D J, Shanley R A, Demirel H, Naito H. Obesity is associated with increased myocardial oxidative stress. Int J Obes Relat Metab Disord. 1999; 23:67-74.
27. Higdon T V, Frei B. Obesity and oxidative stress: a direct link to CVD? Arterioscler. Thromb Vasc Biol. 2003; 23:356-67.
28. Hotamisligil G S, Shargill N S, Spiegelmann B M. Adipose expression of tumor necrosis factor alpha: direct role in obesity-linked insulin resistance. Science. 1993; 259:87-91.
29. Matthews D R, Hosker J P, Rudenski A S et al. Homeostasis model assessment: insulin resistance and beta-cell function from fasting plasma glucose and insulin concentrations in man. Diabetologia. 1985; 28:412-9.
30. Hedblad B, Nilsson P, Janzon L et al. Relation between insulin resistance and carotid intima-media thickness and stenosis in non-diabetic subjects. Results from a cross-sectional study in Malmo, Sweden. Diabet Med. 2000; 17:299-307.
31. Wittwer C, Hahn M, Kaul M. (Eds.) Rapid Cycle Real-Time PCR Methods and Applications Quantification, Springer Verlag, Berlin, 2004
32. Tomiyasu M, Yoshino I, Suemitsu R, Okamoto T, Sugimachi K. Quantification of macrophage migration inhibitory factor mRNA expression in non-small cell lung cancer tissues and its clinical significance. Clin Cancer Res. 2002; 8:3755-60.
33. Nakajima-Iijima S, Hamada H, Reddy P, Kakunaga T. Molecular structure of the human cytoplasmic beta-actin gene: interspecies homology of sequences in the introns. Proc Natl Acad Sci USA. 1985; 82:6133-7.
34. Forst T, Lubben G, Hohberg C et al. Influence of glucose control and improvement of insulin resistance on microvascular blood flow and endothelial function in patients with diabetes mellitus type 2. Microcirculation. 2005; 12:543-50.
35. Forst T, Hohberg C, Fuellert S et al. Pharmacological PPARγ Stimulation in Contrast to Beta Cell Stimulation results in an Improvement in Adiponectin and Proinsulin Intact levels and Reduces Intima Media Thickness in Patients with Type 2 Diabetes. Horm Metab Res. 2005; 37:521-7.
36. Hanefeld M, Marx N, Pfiitzner A et al. Anti-inflammatory effects of pioglitazone and/or simvastatin in high cardiovascular risk patients with elevated high sensitivity C-reactive protein: the PIOSTAT Study. J Am Coll Cardiol. 2007; 49:290-7.
37. Fontana L, Eagon J C, Trujillo M E, Scherer P E, Klein S. Visceral fat adipokine secretion is associated with systemic inflammation in obese humans. Diabetes. 2007; 56:1010-3.
38. Bodles A M, Varma V, Yao-Borengasser A et al. Pioglitazone induces apoptosis of macrophages in human adpose tissue. J Lipid Res. 2006; 47:2080-8.
39. Forst T, Pfützner A, Lubben G et al. Effect of simvastatin and/or pioglitazone on insulin resistance, insulin secretion, adiponectin, and proinsulin levels in nondiabetic patients at cardiovascular risk—the PIOSTAT Study. Metabolism. 2007; 56:491-6.

1. A kit comprising:
(a) a first solid support comprising:
(i) a capture binding ligand selective for hsCRP; and
(b) a second solid support comprising:
(i) a capture probe selective for MCP-1 nucleic acid;
(ii) a capture probe selective for MMP-9 nucleic acid; and
(iii) a capture probe selective for TNFα nucleic acid.

2. The kit of claim 1 wherein the capture binding ligand comprises an antibody.

3. The kit of claim 1 further comprising:
(a) a soluble capture ligand selective for hsCRP;
wherein the soluble capture ligand comprises a detectable label.

4. The kit of claim 1 further comprising:
(a) a label probe selective for MCP-1 nucleic acid;
(b) a label probe selective for MMP-9 nucleic acid; and
(c) a label probe selective for TNFα nucleic acid;
wherein each of the label probes comprises a detectable label.

5. The kit of claim 1 further comprising:
(a) a primer selective for MCP-1 nucleic acid;
(b) a primer selective for MMP-9 nucleic acid; and
(c) a primer selective for TNFα nucleic acid;
wherein each of the primers optionally comprises a detectable label.

6. The kit of claim 5 wherein the detectable label is a fluorophore.

7. The kit of claim 5 wherein the detectable label comprises biotin.

8. The kit of claim 7 further comprising a horseradish peroxidase conjugate.

9. The kit of claim 8 further comprising a precipitating agent.

10.-11. (canceled)

12. A method of treating atherosclerosis in a subject comprising
(a) measuring the concentrations of hsCRP, MCP-1 nucleic acid, MMP-9 nucleic acid and TNFα nucleic acid in a first sample from the subject; and
(b) effecting a first therapy with respect to the subject,
wherein the concentration(s) of one, a combination or all of hsCRP, MCP-1 nucleic acid, MMP-9 nucleic acid and TNFα nucleic acid in a second sample from the subject after the first therapy decrease(s) after effecting the first therapy compared to corresponding concentration(s) in the first sample,
thereby treating atherosclerosis in the subject.

13. (canceled)

14. The method of claim 12 wherein the concentration(s) of one, a combination or all of MCP-1 nucleic acid, MMP-9 nucleic acid and TNFα nucleic acid in a second sample from the subject decrease(s) by at least about 15% compared to corresponding concentration(s) in the first sample.

15. The method of claim 12 wherein the concentration of hsCRP acid in a second sample from the subject decreases by about 10% to about 40% compared to the corresponding concentration in the first sample.

16. The method of claim 12 wherein the first therapy comprises administering a first disease-modulating drug to the subject.

17. A method of assessing the efficacy of a first therapy on a subject experiencing atherosclerosis comprising:
(a) taking a first measurement of the concentrations of hsCRP, MCP-1 nucleic acid, MMP-9 nucleic acid and TNFα nucleic acid in a first sample from the subject;
(b) effecting the first therapy on the subject;
(c) taking a second measurement of the concentrations of hsCRP, MCP-1 nucleic acid, MMP-9 nucleic acid and TNFα nucleic acid in a second sample from the subject after the first therapy; and
(d) making a comparison between the first and second measurements.

18. The method of claim 17 further comprising (e) effecting a second therapy on the subject based on the comparison.

19. The method of claim 18 wherein effecting the first therapy comprises administering a first disease-modulating drug to the subject according to a first dosage regimen.

20. The method of claim 19 wherein effecting a second therapy comprises making a decision regarding the continued administration of the first disease-modulating drug.

21. The method of claim 19 wherein effecting a second therapy comprises administering a second disease-modulating drug to the subject.

22. The method of claim 19 wherein effecting a second therapy comprises administering a statin to the subject.

23. The method of claim 19 wherein effecting a second therapy comprises discontinuing the administration of the first disease-modulating drug.

24. The method of claim 19 wherein effecting a second therapy comprises repeating or maintaining the administration of the first disease-modulating drug.

25. The method of claim 24 wherein effecting a second therapy comprises administering the first disease-modulating drug according to an adjusted dosage regimen compared to the first dosage regimen.

26. The method of claim 25 wherein the adjusted dosage regimen depends on the degree of change in the concentration(s) of one, a combination or all of hsCRP, MCP-1 nucleic acid, MMP-9 nucleic acid and TNFα nucleic acid between the first and second measurement.

27. The method of claim 24 wherein if the concentration(s) of one, a combination or all of MCP-1 nucleic acid, MMP-9 nucleic acid and TNFα nucleic acid decrease(s) by at least about 15% between the first and second measurements, then effecting a second therapy comprises repeating or maintaining the administration of the first disease-modulating drug.

28. The method of claim 24 wherein if the concentration of hsCRP decreases by about 10% to about 40% between the first and second measurement, then effecting a second therapy comprises repeating or maintaining the administration of the first disease-modulating drug.

29. The method of claim 23 wherein if the concentration(s) of one, a combination or all of MCP-1 nucleic acid, MMP-9 nucleic acid and TNFα nucleic acid do(es) not decrease by at least about 15% between the first and second measurements, then effecting a second therapy comprises discontinuing the administration of the first disease-modulating drug.

30. The method of claim 23 wherein if the concentration of hsCRP does not decrease from about 10% to about 40% between the first and second measurements, then effecting a second therapy comprises discontinuing the administration of the first disease-modulating drug.

31. The method of claim 19 wherein the first disease-modulating drug is an insulin sensitizer.

32. The method of claim 31 wherein the insulin sensitizer is a glitazone.

33. The method of claim 32 wherein the glitazone is pioglitazone.

34.-35. (canceled)

36. The method of claim 17 wherein a sample is contacted with the first and/or second solid support of a kit comprising:
(a) a first solid support comprising:
(i) a capture binding ligand selective for hsCRP; and
(b) a second solid support comprising:
(i) a capture probe selective for MCP-1 nucleic acid;
(ii) a capture probe selective for MMP-9 nucleic acid; and
(iii) a capture probe selective for TNFα nucleic acid.

37. A method of acquiring data relating to a sample comprising (a) taking a measurement of the concentrations of hsCRP, MCP-1 nucleic acid, MMP-9 nucleic acid and TNFα nucleic acid in the sample.

38. The method of claim 37 wherein the sample is derived from a subject, optionally wherein the subject is experiencing atherosclerosis.

39. (canceled)

40. The method of claim 37 wherein the sample is contacted with the first and/or second solid support of a kit comprising:
(a) a first solid support comprising:
(i) a capture binding ligand selective for hsCRP; and
(b) a second solid support comprising:
(i) a capture probe selective for MCP-1 nucleic acid;
(ii) a capture probe selective for MMP-9 nucleic acid; and
(iii) a capture probe selective for TNFα nucleic acid.

41. (canceled)

42. Use of the kit of claim 1 to determine whether a subject belongs to a population that would benefit from a second therapy, wherein the subject has undergone a first therapy and wherein the subject is experiencing atherosclerosis.

43. The use of claim 42 comprising
(a) contacting a first sample from the subject with the first and/or second solid support of the kit;
(b) taking a first measurement of the concentrations of hsCRP, MCP-1 nucleic acid, MMP-9 nucleic acid and TNFα nucleic acid in the first sample;
(c) effecting a first therapy on the subject;
(d) contacting a second sample from the subject with the first and/or second solid support of the kit after the first therapy;
(e) taking a second measurement of the concentrations of hsCRP, MCP-1 nucleic acid, MMP-9 nucleic acid and TNFα nucleic acid in the second sample;
(f) making a comparison of the first and second measurements.

44. The use of claim 43 wherein effecting the first therapy comprises administering a first disease-modulating drug to the subject according to a first dosage regimen.

45. The use of claim 44 wherein the second therapy comprises administering a second disease-modulating drug to the subject.

46.-59. (canceled)
